(19) United States
(12) Patent Application Publication
(10) Pub. No.: US 2018/0179167 A1
(43) Pub. Date: Jun. 28, 2018

(54) Nrf2 REGULATORS

(71) Applicants: GlaxoSmithKline Intellectual Property Development Limited, Brentford Middlesex (GB); Astex Therapeutics, Limited, Cambridgeshire (GB)

(72) Inventors: Jeffrey K. KERNS, King of Prussia, PA (US); Tindy LI, King of Prussia, PA (US); Hongxing YAN, King of Prussia, PA (US); Thomas Daniel HEIGHTMAN, Hertfordshire (GB); Alison Jo-Anne WOOLFORD, Cambridgeshire (GB); Charlotte Mary GRIFFITHS-JONES, Cambridgeshire (GB); Hendrika Maria Gerarda WILLEMS, Cambridgeshire (GB)

(21) Appl. No.: 15/735,971

(22) PCT Filed: Jun. 15, 2016
(86) PCT No.: PCT/IB2016/053544

§ 371 Date: Dec. 13, 2017

Related U.S. Application Data
(60) Provisional application No. 62/175,501, filed on Jun. 15, 2015.

(51) Int. Cl. C07D 249/18; A61P 11/00

(52) U.S. Cl. CPC C07D 249/18; A61P 11/00

(57) ABSTRACT

The present invention relates to aryl analogs Formula (I), pharmaceutical compositions containing them and their use as Nrf2 regulators.

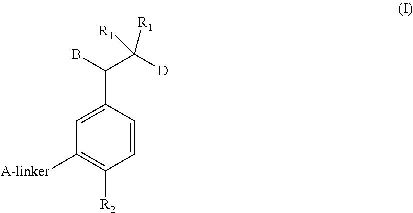

### FIELD OF THE INVENTION

The present invention relates to aryl analogs, pharmaceutical compositions containing them and their use as Nrf2 regulators.

### BACKGROUND OF THE INVENTION

NRF2 (NF-E2 related factor 2) is a member of the cap-n-collar (CNC) family of transcription factors containing a characteristic basic-leucine zipper motif. Under basal conditions, NRF2 levels are tightly controlled by the cytosolic actin-bound repressor, KEAP1 (Kelch-like ECH associating protein 1), which binds to NRF2 and targets it for ubiquitylation and proteasomal degradation via the Cul3-based E3-ubiquitin ligase complex. Under conditions of oxidative stress, DJ1 (PARK7) is activated and stabilizes NRF2 protein by preventing NRF2 from interacting with KEAP1. Also, modification of reactive cysteines on KEAP1 can cause a conformational change in KEAP1 that alters NRF2 binding and promotes NRF2 stabilization. Thus, the levels of NRF2 in the cytosol are low in normal conditions but the system is designed to respond immediately to environmental stress by increasing NRF2 activity.

Inappropriately low NRF2 activity in the face of on-going oxidative stress appears to be a pathological mechanism underlying chronic obstructive pulmonary disease (COPD). This may be a result of an altered equilibrium between NRF2 regulators with both inappropriate lack of positive regulators such as DJ1, and overabundance of negative regulators such as Keap1 and Bach1. Therefore, restoration of NRF2 activity in the lungs of COPD patients should result in repair of the imbalance and mitigation of deleterious processes such as apoptosis of structural cells (including alveolar epithelial and endothelial cells) and inflammation. The results of these effects would be enhanced cytoprotection, preservation of lung structure, and structural repair in the COPD lung, thus slowing disease progression. Therefore, NRF2 modulators may treat COPD (Boutten, A., et al. 2011. Trends Mol. Med. 17:363-371) and other respiratory diseases, including asthma and pulmonary fibrosis (Cho, H. Y., and Kleeberger, S. R. 2010. Toxicol. Appl. Pharmacol. 244:43-56).

An example of inappropriately low NRF2 activity is found in pulmonary macrophages from COPD patients. These cells have impaired bacterial phagocytosis compared with similar cells from control patients, and this effect is reversed by the addition of NRF2 activators in vitro. Therefore, in addition to the effects mentioned above, restoration of appropriate NRF2 activity could also rescue COPD exacerbations by reducing lung infections. This is demonstrated by the NRF2 activator, Sulforaphane, which increases the expression of Macrophage Receptor with Collagenous structure (MARCO) by COPD macrophages and alveolar macrophages from cigarette smoke-exposed mice, thereby improving in these cells bacterial phagocytosis (Pseudomonas aeruginosa, non-typable Haemophilus influenzae) and bacterial clearance both ex vivo and in vivo. (Harvey, C. J., et al. 2011. Sci. Transl. Med. 3:78ra32).

The therapeutic potential of targeting NRF2 in the lung is not limited to COPD. Rather, targeting the NRF2 pathway could provide treatments for other human lung and respiratory diseases that exhibit oxidative stress components such as chronic and acute asthma, lung disease secondary to environmental exposures including but not limited to ozone, diesel exhaust and occupational exposures, fibrosis, acute lung infection (e.g., viral (Noah, T. L. et al. 2014. PLoS ONE 9(6): e98671), bacterial or fungal), chronic lung infection, α1 antitrypsin disease, and cystic fibrosis (C F, Chen, J. et al. 2008. PLoS One. 2008; 3(10):e3367).

A therapy that targets the NRF2 pathway also has many potential uses outside the lung and respiratory system. Many of the diseases for which an NRF2 activator may be useful are autoimmune diseases (psoriasis, IBD, MS), suggesting that an NRF2 activator may be useful in autoimmune diseases in general.

In the clinic, a drug targeting the NRF2 pathway (bardoxolone methyl) has shown efficacy in diabetic patients with diabetic nephropathy/chronic kidney disease (CKD) (Aleksunes, L. M., et al. 2010. J. Pharmacol. Exp. Ther. 335:2-12), though phase III trials with this drug in patients with the most severe stage of CKD were terminated. Furthermore, there is evidence to suspect that such a therapy would be effective in sepsis-induced acute kidney injury, other acute kidney injury (AKI) (Shelton, L. M., et al. 2013. Kidney International. June 19. doi: 10.1038/ki.2013.248.), and kidney disease or malfunction seen during kidney transplantation.

In the cardiac area, bardoxolone methyl is currently under investigation in patients with Pulmonary Arterial Hypertension and so a drug targeting NRF2 by other mechanisms may also be useful in this disease. Also, it may be useful in a variety of cardiovascular diseases including but not limited to atherosclerosis, hypertension, and heart failure (Oxidative Medicine and Cellular Longevity Volume 2013 (2013), Article ID 104308, 10 pages).

A drug activating the NRF2 pathway could also be useful for treatment of several neurodegenerative diseases including Parkinson's disease (PD), Alzheimer's disease (AD), amyotrophic lateral sclerosis (ALS) (Brain Res. 2012 Mar 29; 1446:109-18.2011.12.064. Epub 2012 Jan 12.) and multiple sclerosis (MS). Multiple in vivo models have shown that NRF2 KO mice are more sensitive to neurotoxic insults than their wild-type counterparts. Treatment of rats with the NRF2 activator tert-butylhydroquinone (tBHQ) reduced cortical damage in rats in a cerebral ischemia-reperfusion model, and cortical glutathione levels were increased in NRF2 wild-type but not KO mice after administration of tBHQ (Shih, A. Y.,et al. 2005. J. Neurosci. 25: 10321-10335). Tecfidera™ (dimethyl fumarate), which activates NRF2 among other targets, is approved in the U.S. to treat relapsing-remitting multiple sclerosis (MS). Activation of NRF2 may also help treat cases of Friedreich's Ataxia, where increased sensitivity to oxidative stress and impaired NRF2 activation has been reported (Paupe V., et al, 2009. PLoS One; 4(1):e4253.

There is preclinical evidence of the specific protective role of the NRF2 pathway in models of inflammatory bowel disease (IBD, Crohn's Disease and Ulcerative Colitis) and/or colon cancer (Khor, T. O., et al 2008. Cancer Prev. Res. (Phila) 1:187-191).

Age-related macular degeneration (AMD) is a common cause of vision loss in people over the age of 50. Cigarette smoking is a major risk factor for the development of non-neovascular (dry) AMD and perhaps also neovascular (wet) AMD. Findings in vitro and in preclinical species support the notion that the NRF2 pathway is involved in the anti-oxidant response of retinal epithelial cells and modulation of inflammation in pre-clinical models of eye injury (Schimel, et al. 2011. Am. J. Pathol. 178:2032-2043). Fuchs Endothelial Corneal Dystrophy (FECD) is a progressive, blinding disease characterized by corneal endothelial cells apoptosis. It is a disease of aging and increased oxidative stress related to low levels of NRF2 expression and/or function (Bitar, M. S., et al. 2012. Invest Ophthalmol. Vis. Sci. Aug. 24, 2012 vol. 53 no. 9 5806-5813). In addition, an NRF2 activator may be useful in uveitis or other inflammatory eye conditions.

Non-alcoholic steatohepatitis (NASH) is a disease of fat deposition, inflammation, and damage in the liver that occurs in patients who drink little or no alcohol. In pre-clinical models, development of NASH is greatly accelerated in KO mice lacking NRF2 when challenged with a methionine- and choline-deficient diet (Chowdhry S., et al. 2010. Free Rad. Biol. & Med. 48:357-371). Administration of the NRF2 activators oltipraz and NK-252 in rats on a choline-deficient L-amino acid-defined diet significantly attenuated progression of histologic abnormalities, especially hepatic fibrosis (Shimozono R. et al. 2012. Molecular Pharmacology. 84:62-70). Other liver diseases that may be amenable to NRF2 modulation are toxin-induced liver disease (e.g., acetaminophen-induced hepatic disease), viral hepatitis, and cirrhosis (Oxidative Medicine and Cellular Longevity Volume 2013 (2013), Article ID 763257, 9 page).

Recent studies have also begun to elucidate the role of ROS in skin diseases such as psoriasis. A study in psoriasis patients showed an increase in serum malondialdehyde and nitric oxide end products and a decrease in erythrocyte-superoxide dismutase activity, catalase activity, and total antioxidant status that correlated in each case with disease severity index (Dipali P. K., et al. Indian J Clin Biochem. 2010 October; 25(4): 388-392). Also, an NRF2 modulator may be useful in treating the dermatitis/topical effects of radiation (Schäfer, M. et al. 2010. Genes & Devl. 24:1045-1058), and the immunosuppression due to radiation exposure (Kim J H et al, J. Clin. Invest. 2014 Feb. 3; 124(2):730-41).

There are also data suggesting that an NRF2 activator may be beneficial in preeclampsia, a disease that occurs in 2-5% of pregnancies and involves hypertension and proteinuria (Annals of Anatomy—Anatomischer Anzeiger Volume 196, Issue 5, September 2014, Pages 268-277).

Preclinical data has shown that compounds with NRF2 activating activity are better at reversing high altitude-induced damage than compounds without NRF2 activity, using animal and cellular models of Acute Mountain Sickness (Lisk C. et al, 2013, Free Radic Biol Med. October 2013; 63: 264-273.)

### SUMMARY OF THE INVENTION

In one aspect this invention provides for aryl analogs, pharmaceutically acceptable salts thereof, and pharmaceutical compositions containing them.

In a second aspect, this invention provides for the use of the compounds of Formula (I) as Nrf2 regulators.

In one embodiment, the invention is directed to a pharmaceutical composition comprising a compound of the invention according to Formula (I), or a pharmaceutically acceptable salt thereof, and a pharmaceutically acceptable excipient. Particularly, this invention is directed to a pharmaceutical composition for the treatment of an Nrf2 regulated disease or disorder, wherein the composition comprises a compound according to Formula (I), or a pharmaceutically acceptable salt thereof, and a pharmaceutically acceptable excipient.

In another aspect, this invention provides for the use of the compounds of Formula (I) for treating and preventing conditions associated with Nrf2 imbalance.

In a further aspect, this invention provides for a method of treating respiratory and non-respiratory disorders, including COPD, asthma, fibrosis, chronic asthma, acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye conditions, Non-alcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness, which comprises administering to a human in need thereof, a compound of Formula (I).

In yet another aspect, this invention provides for the use of the compounds of Formula (I) for the treatment of respiratory and non-respiratory disorders, including COPD, asthma, fibrosis, chronic and acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye conditions, Non-alcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness.

In a further aspect, this invention relates to use of a compound of Formula (I) or a pharmaceutically acceptable salt thereof in the manufacture of a medicament for the treatment of respiratory and non-respiratory disorders, including COPD, asthma, fibrosis, chronic and acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye conditions, Non-alcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness.

In a further aspect, this invention relates to a compound of Formula (I) or a pharmaceutically acceptable salt thereof, for use in medical therapy.

In a further aspect, this invention relates to a compound of Formula (I) or a pharmaceutically acceptable salt thereof, for use in the treatment of respiratory and non-respiratory disorders, including COPD, asthma, fibrosis, chronic and acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye conditions, Non-alcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness.

In a further aspect, this invention relates to a compound of Formula (I) or a pharmaceutically acceptable salt thereof, for use in the treatment of COPD.

In a further aspect, this invention relates to a method of treating COPD which comprises administering to a human in need thereof, a compound of Formula (I).

In a further aspect, this invention relates to a method of treating heart failure which comprises administering to a human in need thereof, a compound of Formula (I).

In a further aspect, this invention relates to a compound of Formula (I) or a pharmaceutically acceptable salt thereof, for use in the treatment of heart failure.

The compounds of Formula (I) and pharmaceutically acceptable salts thereof may be used in combination with one or more other agents which may be useful in the prevention or treatment of allergic disease, inflammatory disease, autoimmune disease, for example; antigen immunotherapy, anti-histamines, corticosteroids, (e.g., fluticasone propionate, fluticasone furoate, beclomethasone dipropionate, budesonide, ciclesonide, mometasone furoate, triamcinolone, flunisolide), NSAIDs, leukotriene modulators (e.g., montelukast, zafirlukast, pranlukast), iNOS inhibitors, tryptase inhibitors, IKK2 inhibitors, p38 inhibitors, Syk inhibitors, protease inhibitors such as elastase inhibitors, integrin antagonists (e.g., beta-2 integrin antagonists), adenosine A2a agonists, mediator release inhibitors such as sodium chromoglycate, 5-lipoxygenase inhibitors (zyflo), DP1 antagonists, DP2 antagonists, PI3K delta inhibitors, ITK inhibitors, LP (lysophosphatidic) inhibitors or FLAP (5-lipoxygenase activating protein) inhibitors (e.g. sodium 3-(3-(tert-butylthio)-1-(4-(6-ethoxypyridin-3-yl)benzyl)-5-((5-methylpyridin-2-yl)methoxy)-1H-indol-2-yl)-2,2-dimethylpropanoate), bronchodilators (e.g., muscarinic antagonists, beta-2 agonists), methotrexate, and similar agents; monoclonal antibody therapy such as anti-IgE, anti-TNF, anti-IL-5, anti-IL-6, anti-IL-12, anti-IL-1 and similar agents; cytokine receptor therapies e.g., etanercept and similar agents; antigen non-specific immunotherapies (e.g., interferon or other cytokines/chemokines, chemokine receptor modulators such as CCR3, CCR4 or CXCR2 antagonists, other cytokine/chemokine agonists or antagonists, TLR agonists and similar agents).

The compounds may also be used in combination with agents for aiding transplantation including Cyclosporines, Tacrolimus, Mycophenolate mofetil, Prednisone, Azathioprine, Sirolimus, Daclizumab, Basiliximab, or OKT3.

They may also be used in combination with agents for Diabetes: metformin (biguanides), meglitinides, sulfonylureas, DPP-4 inhibitors, Thiazolidinediones, Alpha-glucosidase inhibitors, Amylin mimetics, Incretin mimetics, and insulin.

The compounds may be used in combination with antihypertensives such as diuretics, ACE inhibitors, ARBS, calcium channel blockers, and beta blockers.

Other aspects and advantages of the present invention are described further in the following detailed description of the preferred embodiments thereof.

In one embodiment, the invention is directed to the use of a compound of Formula (I), or a pharmaceutically acceptable salt thereof, as an active therapeutic substance. More specifically, this invention provides for the use of the compounds described herein for the treatment of a respiratory and non-respiratory disorder, specifically, a disease or disorder recited herein. Accordingly, the invention provides for the use of a compound of Formula (I), or a pharmaceutically acceptable salt thereof, as an active therapeutic substance in the treatment of a human in need thereof with a respiratory and non-respiratory disorder, specifically, a disease or disorder recited herein.

In one embodiment, the invention is directed to a compound described herein, or a pharmaceutically acceptable salt thereof, in the manufacture of a medicament for treatment of a respiratory and non-respiratory disorder, for example the diseases and disorders recited herein. Specifically, the invention further provides for the use of a compound of Formula (I), or a pharmaceutically acceptable salt thereof, in the manufacture of a medicament for use in the treatment of a respiratory and non-respiratory disorder, for example the diseases and disorders recited herein.

### DETAILED DESCRIPTION OF THE INVENTION

The present invention provides for compounds of Formula (I):

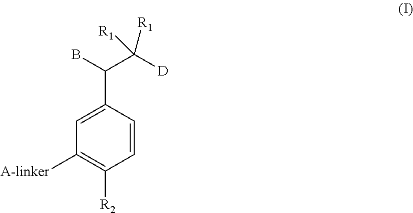

B is benzotriazolyl, phenyl, triazolopyridinyl, or —(CH₂)₂ triazolyl, each of which may be unsubstituted substituted by 1, 2, or 3 substituents independently chosen from: —C₁₋₃alkyl, —O—C₁₋₃alkyl, CN, —(CH₂)₂-—O—(CH₂)₂—OR₄ and halo;
D is —C(O)OH, —C(O)NHSO₂CH₃, —SO₂NHC(O)CH₃, 5-(trifluoromethyl)-4H-1,2,4-triazol-2-yl, or tetrazolyl;
R₁ is independently hydrogen, C₁₋₃ alkyl, F, C₃₋₆spirocycloalkyl, oxetane, or the two R₁ groups together with the carbon they are attached to form a cyclopropyl group;
R₂ is hydrogen, methyl, CF₃, or halo;
Linker is

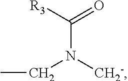

—O—C(O)—N(CH₃)—CH₂—,—C(O)—NH—CH₂— or —N(CH₃)—C(O)—CH₂—O—;
R₃ is CH₃, —(CH₂)₂—OH, or NH₂;
R₄ is hydrogen or C₁₋₃ alkyl;
A is cyclopentyl, cyclohexyl, cycloheptyl, or phenyl each of which may be substituted by one or two of —C₁₋₃ alkyl, CN, halo, —OH, or —O—C₁₋₃ alkyl groups;
or A is C₁₋₅ alkyl which may be substituted by —OCH₃;
and phenyl may also be substituted by —O—CH(CH₃)—C(O)—OH— or NO₂;
or a pharmaceutically acceptable salt thereof.

“Alkyl” refers to a monovalent saturated hydrocarbon chain having the specified number of carbon member atoms. For example, C₁₋₃ alkyl refers to an alkyl group having from 1 to 3 carbon member atoms. Alkyl groups may be straight or branched. Representative branched alkyl groups have one, two, or three branches. Alkyl includes methyl, ethyl, and propyl, (n-propyl and isopropyl).

When used herein, the terms ‘halogen’ and ‘halo’ include fluorine, chlorine, bromine and iodine, and fluoro, chloro, bromo, and iodo, respectively.

“Substituted” in reference to a group indicates that one or more hydrogen atom attached to a member atom within the group is replaced with a substituent selected from the group of defined substituents. It should be understood that the term “substituted” includes the implicit provision that such substitution be in accordance with the permitted valence of the substituted atom and the substituent and that the substitution results in a stable compound (i.e., one that does not spontaneously undergo transformation such as by rearrangement, cyclization, or elimination and that is sufficiently robust to survive isolation from a reaction mixture). When it is stated that a group may contain one or more substituents, one or more (as appropriate) member atoms within the group may be substituted. In addition, a single member atom within the group may be substituted with more than one substituent as long as such substitution is in accordance with the permitted valence of the atom. Suitable substituents are defined herein for each substituted or optionally substituted group.

The term “independently” means that where more than one substituent is selected from a number of possible substituents, those substituents may be the same or different. That is, each substituent is separately selected from the entire group of recited possible substituents.

The invention also includes various isomers of the compounds of Formula (I) and mixtures thereof. “Isomer” refers to compounds that have the same composition and molecular weight but differ in physical and/or chemical properties. The structural difference may be in constitution (geometric isomers) or in the ability to rotate the plane of polarized light (stereoisomers). The compounds according to Formula (I) contain one or more asymmetric centers, also referred to as chiral centers, and may, therefore, exist as individual enantiomers, diastereomers, or other stereoisomeric forms, or as mixtures thereof. All such isomeric forms are included within the present invention, including mixtures thereof.

Chiral centers may also be present in a substituent such as an alkyl group. Where the stereochemistry of a chiral center present in Formula (I), or in any chemical structure illustrated herein, is not specified the structure is intended to encompass any stereoisomer and all mixtures thereof. Thus, compounds according to Formula (I) containing one or more chiral centers may be used as racemic mixtures, enantiomerically enriched mixtures, or as enantiomerically pure individual stereoisomers.

Individual stereoisomers of a compound according to Formula (I) which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

As used herein, “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

The skilled artisan will appreciate that pharmaceutically acceptable salts of the compounds according to Formula (I) may be prepared. These pharmaceutically acceptable salts may be prepared in situ during the final isolation and purification of the compound, or by separately treating the purified compound in its free acid or free base form with a suitable base or acid, respectively.

In certain embodiments, compounds according to Formula (I) may contain an acidic functional group and are, therefore, capable of forming pharmaceutically acceptable base addition salts by treatment with a suitable base. Examples of such bases include a) hydroxides, carbonates, and bicarbonates of sodium, potassium, lithium, calcium, magnesium, aluminum, and zinc; and b) primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to Formula (I) may contain a basic functional group and are therefore capable of forming pharmaceutically acceptable acid addition salts by treatment with a suitable acid. Suitable acids include pharmaceutically acceptable inorganic acids and organic acids. Representative pharmaceutically acceptable acids include hydrogen chloride, hydrogen bromide, nitric acid, sulfuric acid, sulfonic acid, phosphoric acid, acetic acid, hydroxyacetic acid, phenylacetic acid, propionic acid, butyric acid, valeric acid, maleic acid, acrylic acid, fumaric acid, succinic acid, malic acid, malonic acid, tartaric acid, citric acid, salicylic acid, benzoic acid, tannic acid, formic acid, stearic acid, lactic acid, ascorbic acid, methylsulfonic acid, p-toluenesulfonic acid, oleic acid, lauric acid, and the like.

As used herein, the term “a compound of Formula (I)” or “the compound of Formula (I)” refers to one or more compounds according to Formula (I). The compound of Formula (I) may exist in solid or liquid form. In the solid state, it may exist in crystalline or noncrystalline form, or as a mixture thereof. The skilled artisan will appreciate that pharmaceutically acceptable solvates may be formed from crystalline compounds wherein solvent molecules are incorporated into the crystalline lattice during crystallization. Solvates may involve non-aqueous solvents such as, but not limited to, ethanol, isopropanol, DMSO, acetic acid, ethanolamine, or ethyl acetate, or they may involve water as the solvent that is incorporated into the crystalline lattice. Solvates wherein water is the solvent incorporated into the crystalline lattice are typically referred to as “hydrates.” Hydrates include stoichiometric hydrates as well as compositions containing variable amounts of water. The invention includes all such solvates.

The skilled artisan will further appreciate that certain compounds of the invention that exist in crystalline form, including the various solvates thereof, may exhibit polymorphism (i.e. the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The subject invention also includes isotopically-labelled compounds, which are identical to those recited in Formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labelled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula (I) and following of this invention can generally be prepared by carrying out the procedures disclosed in the Schemes and/or in the Examples below, by substituting a readily available isotopically labeled reagent for a non-isotopically labelled reagent.

### Representative Embodiments

In one embodiment for Formula (I):
B is benzotriazolyl, phenyl, triazolopyridinyl, or —(CH₂)₂ triazolyl, each of which may be unsubstituted or substituted by 1, 2, or 3 substituents independently chosen from:
—C₁₋₃alkyl, —O—C₁₋₃alkyl, CN, —(CH₂)₂—O—(CH₂)₂—OR₄ and/or halo;
D is —C(O)OH, —C(O)NHSO₂CH₃, —SO₂NHC(O)CH₃, 5-(trifluoromethyl)-4H-1,2,4-triazol-2-yl, or tetrazolyl;
R₁ is independently hydrogen, —C₁₋₃ alkyl, F, C₃₋₆spirocycloalkyl, oxetane, or the two R₁ groups together with the carbon they are attached to form a cyclopropyl group;
R₂ is hydrogen, methyl, CF₃, or halo;
Linker is

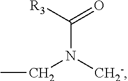

—O—C(O)—N(CH₃)—CH₂—, —C(O)—NH—CH₂— or —N(CH₃)—C(O)—CH₂—O—;
R₃ is CH₃, —(CH₂)₂—OH—, or NH₂;
R₄ is hydrogen or —C₁₋₃alkyl;
A is cyclopentyl, cyclohexyl, cycloheptyl, or phenyl each of which may be substituted independently by one or two of
—C₁₋₃ alkyl, CN, halo, —OH, or —O—C₁₋₃ alkyl groups;
or A is —C₁₋₅ alkyl which may be substituted by —OCH₃;
and wherein if A is phenyl, it may also be substituted by —O—CH(CH₃)—C(O)—OH— or NO₂;
or a pharmaceutically acceptable salt thereof.

In another embodiment for Formula (I):
B is benzotriazolyl or —(CH₂)₂ triazolyl, each of which may be unsubstituted or substituted by 1, 2, or 3 substituents independently chosen from:
—C₁₋₃alkyl, —O—C₁₋₃alkyl and/or halo;
D is —C(O)OH;
R₁ is independently hydrogen or methyl or the two R₁ groups together with the carbon they are attached to form a cyclopropyl group;
R₂ is methyl or chloro;
Linker is

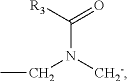

—O—C(O)—N(CH₃)—CH₂ or —N(CH₃)—C(O)—CH₂—O—;
R₃ is CH₃, —(CH₂)₂—OH—, or NH₂;
A is cyclopentyl or cyclohexyl, cycloheptyl, each of which may be substituted independently by one or two —C₁₋₃ alkyl or —OH;
or a pharmaceutically acceptable salt thereof.

In another embodiment for Formula (I):
B is benzotriazolyl which may be unsubstituted or substituted by 1, 2, or 3 substituents independently chosen from:
—C₁₋₃alkyl and/or —O—C₁₋₃alkyl;
D is —C(O)OH;
R₁ is hydrogen;
R₂ is methyl or halo;
Linker is

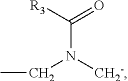

—O—C(O)—N(CH₃)—CH₂—, or —N(CH₃)—C(O)—CH₂—O—;
R₃ is CH₃, —(CH₂)₂—OH—, or NH₂;
A is cyclopentyl, cyclohexyl, cycloheptyl, or phenyl each of which may be substituted independently by one or two of —C₁₋₃ alkyl, CN, halo, —OH, or —O—C₁₋₃ alkyl groups;
or A is —C₁₋₅ alkyl which may be substituted by —OCH₃;
and, wherein A is phenyl, it may also be substituted independently by —O—CH(CH₃)—C(O)—OH— or NO₂;
or a pharmaceutically acceptable salt thereof.

It is to be understood that the present invention covers all combinations of particular groups described hereinabove.

Specific examples of compounds of the present invention include the following:
3-[3-({[(tert-butoxy)carbonyl]methyl)amino}methyl)-4-chlorophenyl]-3-(1,4-dimethyl-1H-1,2,3-benzotriazol-5-yl)propanoic acid;
3-[4-chloro-3-({[(cyclopentyloxy)carbonyl](methyl)amino}methyl)phenyl]-3-(1-methyl-1H-1,2,3-benzotriazol-5-yl)propanoic acid;
3-[3-({[(butan-2-yloxy)carbonyl](methyl)amino}methyl)-4-chlorophenyl]-3-(1-methyl-1H-1,2,3-benzotriazol-5-yl)propanoic acid;
3-[3-({[(tert-butoxy)carbonyl]methyl)amino}methyl)-4-methylphenyl]-3-(7-methoxy-1-methyl-1H-1,2,3-benzotriazol-5-yl)propanoic acid;
3-[4-chloro-3-({[(cyclohexyloxy)carbonyl](methyl)amino}methyl)phenyl]-3-(1-methyl-1H-1,2,3-benzotriazol-5-yl)propanoic acid;
3-[3-({[(tert-butoxy)carbonyl]methyl)amino}methyl)-4-chlorophenyl]-3-(7-methoxy-1-methyl-1H-1,2,3-benzotriazol-5-yl)propanoic acid;
3-{3-[(dimethylcarbamoyl)methoxy]-4-methylphenyl}-3-(1-methyl-1H-1,2,3-benzotriazol-5-yl)propanoic acid;
3-(1,4-dimethyl-1H-1,2,3-benzotriazol-5-yl)-3-(4-methyl-3-{[methyl(4-nitrophenoxycarbonyl)amino]methyl}phenyl)propanoic acid;
3-[4-chloro-3-({[(cyclopentyloxy)carbonyl](methyl)amino}methyl)phenyl]-3-(7-methoxy-1-methyl-1H-1,2,3-benzotriazol-5-yl)propanoic acid;
3-(1,4-dimethyl-1H-1,2,3-benzotriazol-5-yl)-3-{3-[({[(1-methoxy-2-methylpropan-2-yl)oxy]carbonyl}(methyl)amino)methyl]-4-methylphenyl}propanoic acid;
3-(3-{[N-(cyclohexylmethyl)acetamido]methyl}-4-methylphenyl)-3-(1,4-dimethyl-1H-1,2,3-benzotriazol-5-yl)propanoic acid;
3-{3-[(N-benzylacetamido)methyl]-4-methylphenyl}-3-(1,4-dimethyl-1H-1,2,3-benzotriazol-5-yl)propanoic acid;
3-(1,4-dimethyl-1H-1,2,3-benzotriazol-5-yl)-3-[4-methyl-3-({N-[(3-methylphenyl)methyl]acetamido}methyl)phenyl]propanoic acid;
3-(1,4-dimethyl-1H-1,2,3-benzotriazol-5-yl)-3-[3-({N-[(2,3-dimethylphenyl)methyl]acetamido}methyl)-4-methylphenyl]propanoic acid;
3-(1,4-dimethyl-1H-1,2,3-benzotriazol-5-yl)-3-[3-({N-[(4-methoxyphenyl)methyl]acetamido}methyl)-4-methylphenyl]propanoic acid;
3-(1,4-dimethyl-1H-1,2,3-benzotriazol-5-yl)-3-[3-({N-[(4-ethylphenyl)methyl]acetamido}methyl)-4-methylphenyl]propanoic acid;
3-(1,4-dimethyl-1H-1,2,3-benzotriazol-5-yl)-3-[3-({N-[(4-ethylcyclohexyl)methyl]acetamido}methyl)-4-methylphenyl]propanoic acid;
3-(3-{[carbamoyl(cyclohexylmethyl)amino]methyl}-4-methylphenyl)-3-(1,4-dimethyl-1H-1,2,3-benzotriazol-5-yl)propanoic acid;
3-[3-({carbamoyl[(4-ethylcyclohexyl)methyl]amino}methyl)-4-methylphenyl]-3-(1,4-dimethyl-1H-1,2,3-benzotriazol-5-yl)propanoic acid;
3-(3-{[N-(cyclohexylmethyl)acetamido]methyl}-4-methylphenyl)-3-(7-methoxy-1-methyl-1H-1,2,3-benzotriazol-5-yl)propanoic acid;
2-{2-[({5-[2-carboxy-1-(1,4-dimethyl-1H-1,2,3-benzotriazol-5-yl)ethyl]-2-methylphenyl}methyl)carbamoyl]phenoxy}propanoic acid;
3-(1,4-Dimethyl-1H-benzo[d][1,2,3]triazol-5-yl)-3-(3-((1-((4-ethylcyclohexyl)methyl)ureido)methyl)-4-methylphenyl)-2,2-dimethylpropanoic acid;
(S)-3-(3-((1-(Cycloheptylmethyl)ureido)methyl)-4-methylphenyl)-3-(1,4-dimethyl-1H-benzo[d][1,2,3]triazol-5-yl)-2,2-dimethylpropanoic acid;
(S)-3-(1,4-Dimethyl-1H-benzo[d][1,2,3]triazol-5-yl)-3-(3-((1-((4-ethyl-1-hydroxycyclohexyl)methyl)ureido)methyl)-4-methylphenyl)-2,2-dimethylpropanoic acid;
(S)-3-(1,4-Dimethyl-1H-benzo[d][1,2,3]triazol-5-yl)-3-(3-((1-((1-hydroxycycloheptyl)methyl)ureido)methyl)-4-methylphenyl)-2,2-dimethylpropanoic acid;
3-(1,4-Dimethyl-1H-benzo[d][1,2,3]triazol-5-yl)-3-(3-((N-((1-hydroxycyclohexyl)methyl)acetamido)methyl)-4-methylphenyl)-2,2-dimethylpropanoic acid; and
3-(3-((N-(Cycloheptylmethyl)acetamido)methyl)-4-methylphenyl)-3-(1,4-dimethyl-1H-benzo[d][1,2,3]triazol-5-yl)propanoic acid;
or a pharmaceutically acceptable salt thereof.

### Compound Preparation

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

The synthesis of the compounds of the general Formula (I) and pharmaceutically acceptable derivatives and salts thereof may be accomplished as outlined below in Schemes 1-6. In the following description, the groups are as defined above for compounds of Formula (I) unless otherwise indicated. Abbreviations are as defined in the Examples section. Starting materials are commercially available or are made from commercially available starting materials using methods known to those skilled in the art.

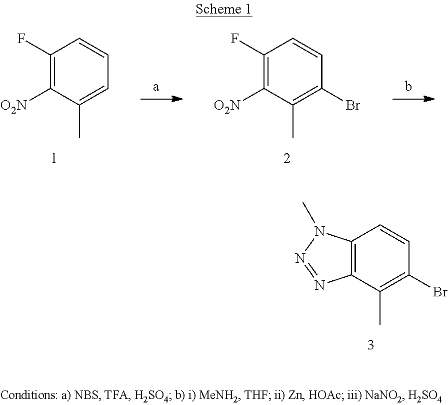

Scheme 1 shows a general scheme for the preparation of 5-bromo-4-methyl-1-methyl-1H-benzo[d][1,2,3]triazole. Starting with commercially available 1-fluoro-3-methyl-2-nitrobenzene, bromination with NBS provides intermediate 2. Displacement of the fluoride using an appropriate amine followed by zinc metal reduction of the nitro to the aniline and diazotization and cyclization provides the required triazole 3. Completion of the fully elaborated analog can be accomplished in a fashion analogous to that shown in schemes 4 and 5.

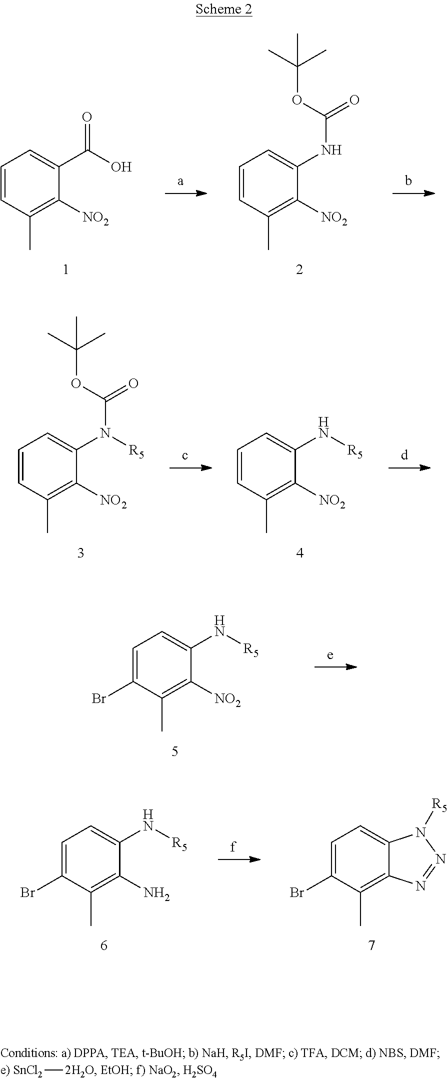

Scheme 2 shows an alternate general scheme for the preparation of 5-bromo-4-methyl-1-methyl-1H-benzo[d][1,2,3]triazole. In scheme 2, R₅ is C₁₋₃alkyl or —(CH₂)₂—O—(CH₂)₂—OR₄. Starting with commercially available 3-methyl-2-nitrobenzoic acid, a Curtius rearangement with DPPA provides intermediate 2. A skilled artisan will appreciate that compound 2 could be prepared from the appropriate aniline compound. Alkylation of the carbamate with an alkyl iodide provides intermediate 3. Deprotection of the amine with TFA and bromination with NBS provides intermediate 5. Reduction of the nitro to the aniline and diazotization and cyclization provides the required triazole 7.

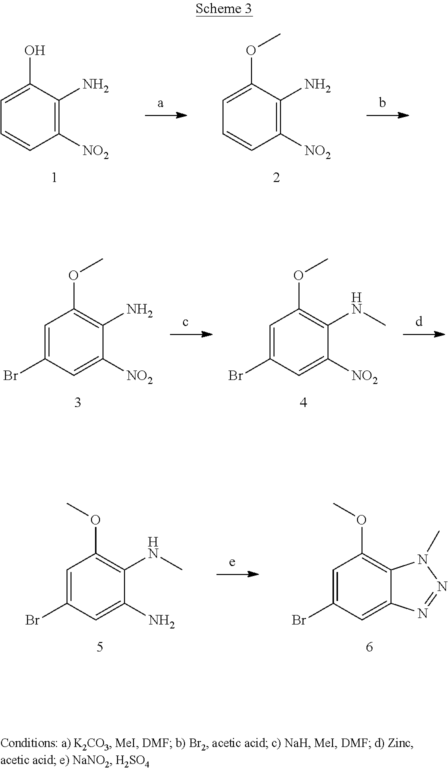

Scheme 3 shows a general scheme for the preparation of 5-bromo-7-methoxy-1-methyl-1H-benzo[d][1,2,3]triazole. Starting with commercially available 2-amino-3-nitrophenol, methylation of the phenol using K₂CO₃ and Mel (step a) provides intermediate 2 which can be brominated with NBS (step c). Methylation of the aniline (step d) followed by reduction of the nitro group (step d) and diazotization and cyclization (step e) provide the required triazole 5. Completion of the fully elaborated analog can be accomplished in a fashion analogous to that shown in schemes 4 and 5.

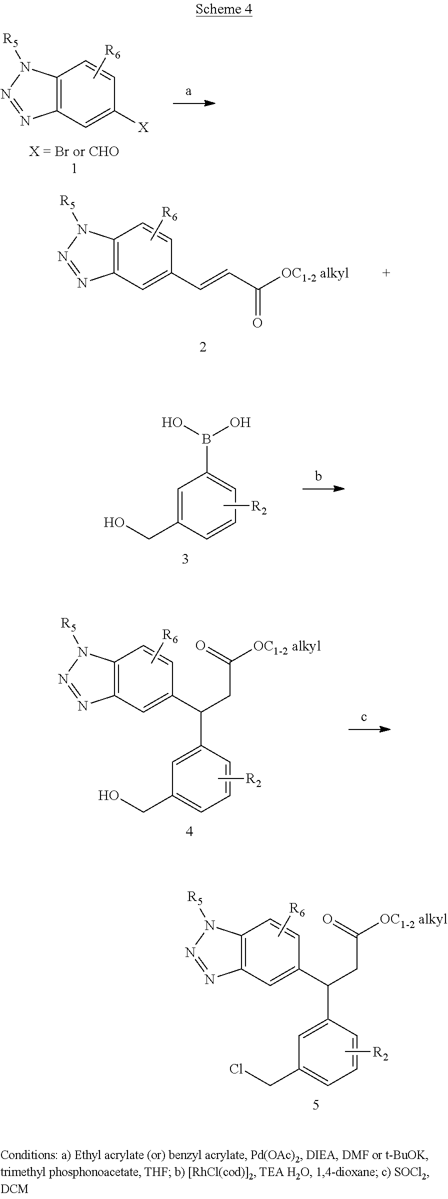

Scheme 4 represents a general scheme for the preparation of compounds according to Formula 1. In scheme 4 R₅ is C₁₋₃alkyl or —(CH₂)₂—O—(CH₂)₂—OR₄ (as defined in Formula 1). R₆ is H, C₁₋₃alkyl, halo, CF₃ or —OC₁₋₃alkyl, R₂ (as defined in Formula 1). The triazole 1 depicted as starting material may be synthesized from readily available materials. Reaction conditions are as described above in the scheme; however, the skilled artisan will appreciate that certain modifications in the reaction conditions and/or reagents used are possible.

Treatment of triazole 1 with ethyl acrylate or benzyl acrylate in the presence of palladium (II) acetate and diisopropylethyl amine in the presence of a suitable solvent produces the desired Heck cross-coupling product 2. It will appreciated by the skilled artisan that other acrylates may be used for the Heck cross-coupling and that compound 2 may also be obtained via a Wittig olefination reaction starting from the appropriate aldehyde of compound 1. Further transformation of the olefin 2 can be achieved through rhodium mediated cross-coupling of the appropriate boronic acid or boronic ester 3 in the presence of triethylamine. It will be recognized by the skilled artisan that the conditions for this Rh catalyzed Michael reaction may be modified by the appropriate selection of ligands, Rh source, solvent and temperature in order to achieve enantioselectivity wherein the chirality at the carbon β to the carboxylate may favor one or the other of the possible enantiomers. Benzylic alcohol 4 can be transformed to the requisite chloride 5 using thionyl chloride.

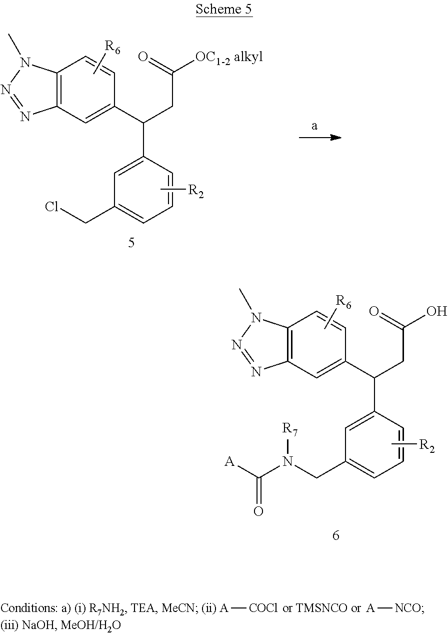

Scheme 5 represents a general scheme for the preparation of compounds according to Formula 1. In Scheme 5 R₆ and R₂ are defined previously, R₇ is hydrogen or methyl and A is as defined in Formula 1. The starting material chloride depicted as starting material 5 can be synthesized as described above. Reaction conditions are as described above in Scheme 5 however, the skilled artisan will appreciate that certain modifications in the reaction conditions and/or reagents used are possible.

The desired acid 6 is prepared in a three step sequence involving reaction of the chloride with

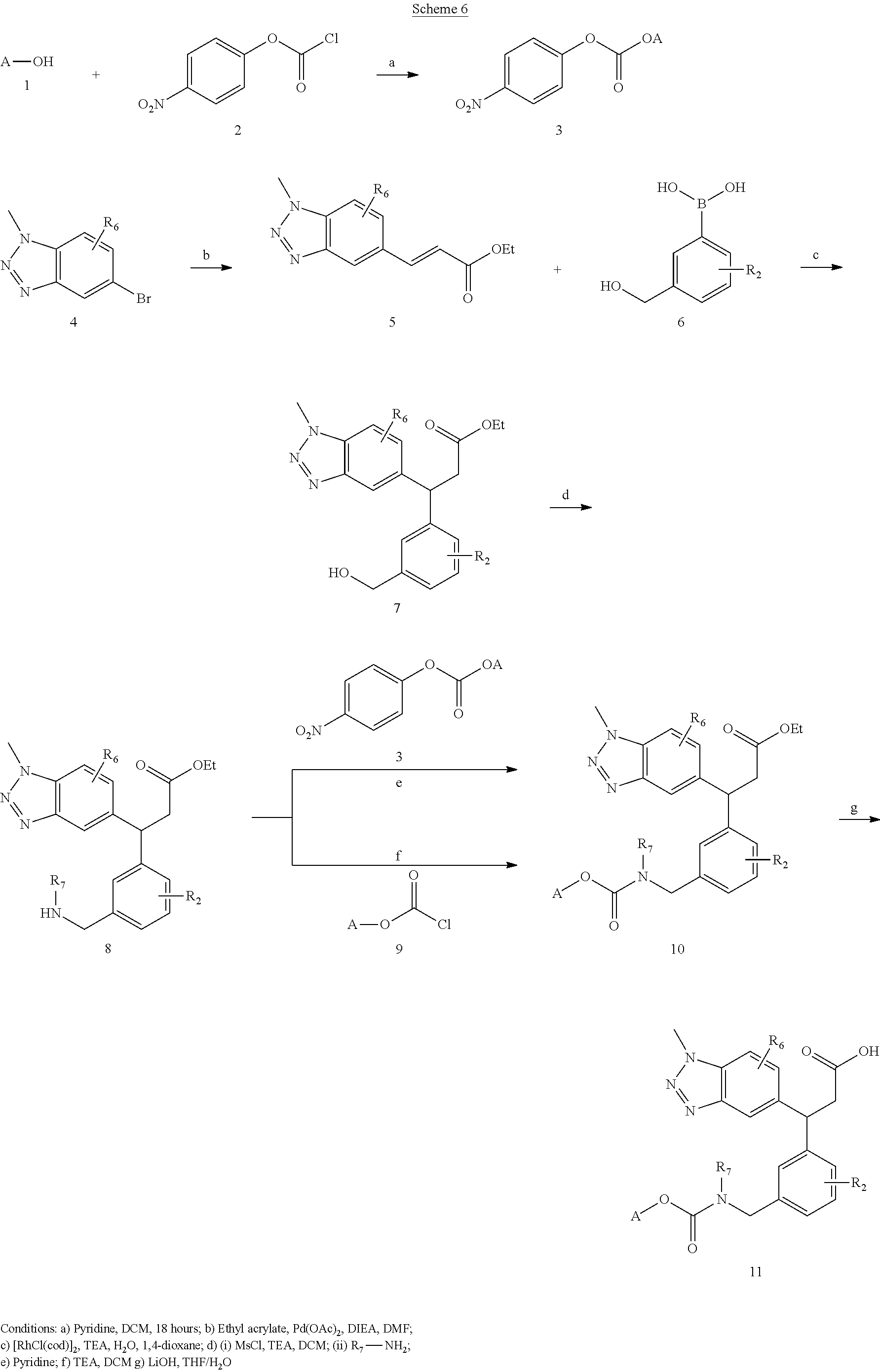

the requisite amine, acylation, and conversion of the ester to the acid.

Scheme 6 represents a general scheme for the preparation of compounds according to Formula 1. In Scheme 6 R₂, R₆, R₇, and A are defined previously. The triazole 4 depicted as starting material may be synthesized from readily available materials. Reaction conditions are as described above in Scheme 6; however, the skilled artisan will appreciate that certain modifications in the reaction conditions and/or reagents used are possible.

Treatment of triazole 4 with ethyl acrylate in the presence of palladium (II) acetate and diisopropylethyl amine in presence of a suitable solvent produces the desired Heck cross-coupling product 5. It will appreciated by the skilled artisan that compound 5 may also be obtained via a Wittig olefination reaction starting from the appropriate aldehyde of compound 4. Further transformation of the olefin 5 can be achieved through rhodium mediated cross-coupling of the appropriate boronic acid or boronic ester 6 in the presence of triethylamine. Conversion of the benzylic alcohol to the amine is accomplished via mesylation and reaction with the appropriate amine to yield 8. Installation of the carbamate and completion of the synthesis can be achieved via reaction with the appropriate chloroformate or p-nitrophenylcarbonate and hydrolysis of the ester.

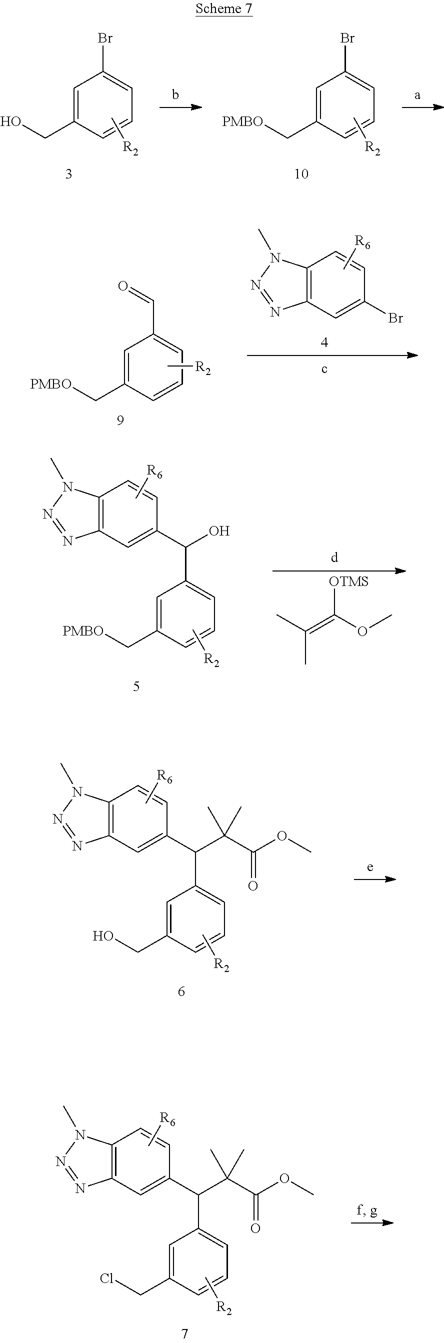

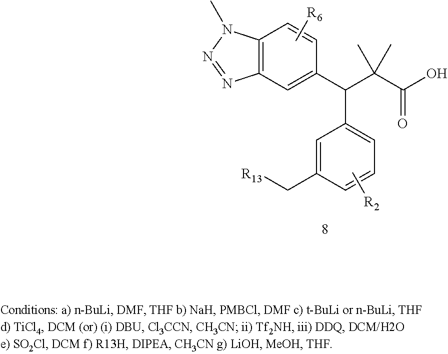

Scheme 7 represents a general scheme for the preparation of compounds according to Formula (I). In Scheme 7, R₂, R₆ and A are as defined previously. R₁₃ is A or A-linker as in Formula (I). Triazole 4 is either commercially available or may be synthesized from readily available materials. Reaction conditions are as described above in Scheme 7; however, the skilled artisan will appreciate that certain modifications in the reaction conditions and/or reagents used are possible.

The preparation of alcohol 5 achieved by first protecting the benzylic alcohol 3 as its paramethoxybenzylether. It will be appreciated that alternative protecting groups are possible. Treatment of bromide 10 with n-butyl lithium and DMF in presence of a suitable solvent produces the desired aldehyde product 9. Coupling of the aldehyde 9 and bromide 4 can be accomplished via treatment of the bromide first with t-butyl lithium or n-butyl lithium followed by addition of the aldehyde. Alternatively, it will be appreciated by one skilled in the art, that the aryl bromide of 9 and the corresponding aldehyde of 4 could be coupled in a similar manner. Intermediate alcohol 6, arises from treatment of alcohol 5 with the appropriate silylketene acetal in the presence of a Lewis acid or via one-pot Bronsted base/Bronsted acid system, followed by deprotection with DDQ. Benzylic alcohol 6 can be transformed to the requisite chloride 7 using thionyl chloride. Completion of the synthesis can be accomplished by deplacement of chloride, followed by hydrolysis of the ester to produce 8.

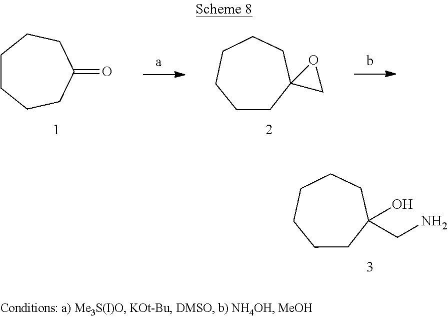 [SMILES: O=C1CCCCCC1 ; C1CCCC2(CC1)CO2 ; NCC1(O)CCCCCC1]

Scheme 8 shows a general scheme for the preparation of 1-(aminomethyl)cycloheptanol. Starting with commercially available cycloheptanone 1, epoxidation is afforded using trimethyl sulfoxonium iodide and potassium tert-butoxide in DMSO, (step a) to provide intermediate 2. Aminolysis is then performed on intermediate 2 using ammonium hydroxide (step b) to provide compound 3.

### Biological Activity

As stated above, the compounds according to Formula I are Nrf2 regulators, and are useful in the treatment or prevention of human diseases that exhibit oxidative stress components such as respiratory and non-respiratory disorders, including COPD, asthma, fibrosis, chronic and acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye condtions, Nonalcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness.

The biological activity of the compounds according to Formula I can be determined using any suitable assay for determining the activity of a candidate compound as a Nrf2 antagonist, as well as tissue and in vivo models.

The biological activity of the compounds of Formula (I) are demonstrated by the following tests.

### BEAS-2B NQO1 MTT Assay

NAD(P)H:quinone oxidoreductase 1 (NQO1), also called DT diaphorase, is a homodimeric FAD-containing enzyme that catalyzes obligatory NAD(P)H-dependent two-electron reductions of quinones and protects cells against the toxic and neoplastic effects of free radicals and reactive oxygen species arising from one-electron reductions. The transcription of NQO1 is finely regulated by Nrf2, and thus NQO1 activity is a good marker for Nrf2 activation. On day one, frozen BEAS-2B cells (ATCC) are thawed in a water bath, counted, and re-suspended at a concentration of 250,000 cells/mL. Fifty microliters of cells are plated in 384 well black clear-bottomed plates. Plates are incubated at 37° C., 5% CO₂ overnight. On day two, plates are centrifuged and 50 nL of compound or controls are added to the cells. Plates are then incubated at 37° C., 5% CO₂ for 48 hours. On day four, medium is aspirated from the plate and crude cell lysates are made by adding 13 uL of 1× Cell Signaling Technologies lysis buffer with 1 Complete, Mini, EDTA-free Protease Inhibitor Tablet (Roche) for each 10 mL of lysis buffer . After lysis plates are incubated for 20 minutes at room temperature. Two microliters of lysate are removed for use in Cell Titer Glo assay (Promega) and MTT cocktail is prepared (Prochaska et. al. 1998) for measurement of NQO1 activity. Fifty microliters of MTT cocktail is added to each well, plate is centrifuged, and analyzed on an Envision plate reader (Perkin Elmer) using Absorbance 570 nm label for 30 minutes. Product formation is measured kinetically and the EC₅₀ of NQO1 specific activity induction is calculated by plotting the change in absorbance (Delta OD/min) versus the log of compound concentration followed by 3-parameter fitting.

All examples described herein possessed NQO1 specific enzyme activity in BEAS-2B cells with EC₅₀s between >10 uM-<1 nM unless otherwise noted (see table below). EC₅₀s <1 nM (+++++), EC₅₀s 10 nM-1 nM (++++), EC₅₀s 10-100 nM (+++), EC₅₀s 100 nM-1 uM (++), EC₅₀s 1-10 uM (+), EC₅₀s >10 uM (−), or were not determined (ND).

| Ex # | EC50 | Ex # | EC50 | Ex # | EC50 |
|---|---|---|---|---|---|
| 1 | ++ | 8 | + | 15 | + |
| 2 | + | 9 | + | 16 | + |
| 3 | + | 10 | + | 17 | +++ |
| 4 | + | 11 | + | 18 | + |
| 5 | + | 12 | + | 19 | + |
| 6 | ++ | 13 | + | 20 | ++ |
| 7 | + | 14 | + | 21 | + |
| 22 | ++++ | 23 | ++++ | 24 | +++ |
| 25 | ++ | 26 | ++ | 27 | ++ |

### Nrf2-Keap1 FP Assay

One model for the Nrf2-Keap1 interaction is through two binding sites in the Neh2 domain on Nrf2. The two sites are referred to as the DLG binding motif (latch domain, uM affinity) and the ETGE binding motif (hinge domain, nM affinity). The Keap1 protein consists of an N-terminal region (NTR), a broad complex, tramtrack, and brick a′ brac domain (BTB), an intervening region (IVR), a double glycine repeat domain (DGR or Kelch), and a C-terminal region. The DLG and ETGE motifs of Nrf2's Neh2 domain bind to the Kelch domain of Keap1 at different affinities. In the Keap1 Kelch fluorescence polarization (FP) assay, a TAMRA-labeled 16 mer peptide (AFFAQLQLDEETGEFL) containing the ETGE motif of Nrf2 and the Kelch domain (321-609) of Keap1 is used. The assay determines if a compound interferes with the binding between Keap1 (361-609) and the TAMRA-labeled peptide. Binding of TAMRA-labeled Nrf2 peptide to Keap1 (321-609) results in a high FP signal. If a compound interferes with the binding between the peptide and the protein, it will cause the assay signal to decrease. Thus, assay signal is inversely proportional to binding inhibition.

FP Assay

100 nl of 100× compound dose response curves (serial 3-fold dilutions) in DMSO are stamped using an Echo liquid handling system (Labcyte) into 384-well low volume black assay plates (Greiner, #784076), with DMSO in columns 6 and 18. The top concentration of compound is located in columns 1 and 13. Keap1 (321-609) is diluted to 40 nM (2×) in 1× assay buffer (50 mM Tris, pH 8.0, 100 mM NaCl, 5 mM MgCl₂, 1 mM DTT, 2 mM CHAPS, and 0.005% BSA) and 5 ul is added using a Multidrop Combi (Thermo Electron Corporation) equipped with a metal tip dispenser to all wells of the compound plate, except column 18. Column 18 receives only 5 ul of assay buffer. Immediately, 5 uL of 16 nM (2×) of Tamra labeled peptide (AFFAQLQLDEETGEFL, 21^{st} Century Biochemicals) is added to all wells of the plate. The plates are spun at 500 rpm for 1 min, incubated for 1 hr at room temperature, and read on an Analyst GT (Molecular Devices) equipped with excitation (530/25 nm) and emission (580/10 nm) filters designed for Tamra probes. A 561 nm dichroic mirror is also used in the Analyst. The final assay concentrations of Keap1 (321-609) and Tamra labeled peptide are 20 nM and 8 nM, respectively. Fluorescence measurements, represented as mP, are used in the transformation of the data. Compound activity is calculated based on percent inhibition, normalized against controls in the assay (Control 1 contains the Tamra peptide and Keap1 (321-609) together (0% response) and control 2 contains the Tamra peptide alone (100% response)). Data analysis is handled using the software package Abase XE (Surrey, United Kingdom. The % inhibition values are calculated by the equation:

100-(100*((compound response-average control 2)/(average control 1-average control2))).For calculation of plC₅₀s, Abase XE uses a four parameter equation.

All examples described herein possessed activity in the Keap1/Nrf2 FP assay.

### Nrf2-Keap1 TR-FRET Assay

In the Nrf2-Keap1 TR-FRET (time-resolved fluorescence resonance energy transfer) assay, full length Nrf2 protein and full length Keap1 protein (Keap1 exists a dimer) are used. The assay detects the ability of compound to displace the binding of FlagHis-tagged Keap1 with biotinylated, Avi-tagged Nrf2 protein. Biotin-Nrf2 binds to streptavidin-europium (a component of the detection mix) and Keap1-FlagHis is recognized by anti-Flag APC (allophycocyanin) antibody (also a component of the detection mix). If binding occurs between the two proteins, there will be an energy transfer from the Eu+3 (donor) at 615 nm to the APC (acceptor) at 665 nm. A potential Keap1 inhibitor will cause a reduction in the TR-FRET signal by interfering with the binding of Keap1 to Nrf2.

One hundred nanoliters of 100× compound dose response curves (serial 3-fold dilutions) in DMSO are stamped using an Echo liquid handling system (Labcyte) into 384-well, low volume, black assay plates (Greiner, #784076), with DMSO in columns 6 and 18. The top concentration of compound is located in columns 1 and 13. All reagents are diluted in assay buffer (50 mM Tris, pH 8.0, 5 mM MgCl2, 100 mM NaCl, 0.005% BSA, 1 mM DTT, and 2 mM CHAPS). The BSA, DTT, and CHAPS are added to the assay buffer on the day of assay. Using a Multidrop Combi (Thermo Electron Corporation) equipped with a metal tip dispenser, 5 ul of 25 nM Keap1-FlagHis protein is added to all wells of the compound plate, with the exception of the wells in column 18. Wells in column 18 receive 5 ul of assay buffer instead. Plates are centrifuged at 500 rpm for 1 minute, covered with a plate lid, and incubated at 37° C. for 2.25 hours. Plates are then removed from the incubator and allowed to cool to RT for 15 minutes. Five microliters of 50 nM biotin-Nrf2 protein is then added to all wells of the plates and the plates are spun at 500 rpm for 1 minute, followed by incubating at 4° C. for 1.25 hours. The plates are then allowed to warm to RT for 15 minutes, followed by the addition of 10 ul of detection mix (1 nM Streptavidin Eu+ W1024 and 5 ug/ml mouse anti-DYKDDDDK IgG conjugated to SureLight APC antibody; both from Columbia Biosciences) to all wells. Plates are spun at 500 rpm for 1 minute, incubated for 1 hour at RT, and read on an Envision plate reader using a 320 nm excitation filter and 615 nm and 665 nm emission filters. Compound response (% inhibition) and potency (plC50) are calculated based on the ratio of the two emissions (665 nm/615 nm) and then the transformed data is normalized against controls in the assay (control 1=1% DMSO in the presence of Nrf2 and Keap1 protein and control 2=1% DMSO in the absence of protein). Data analysis is handled using the software package Abase XE (Surrey, United Kingdom). The % inhibition values are calculated from the ratio (transformed) data by the equation:

100-(100*(compound response-average control 2)/(average control 1-average contro12)).
For calculation of plC50s, Abase XE uses a four parameter equation.

In the Nrf2-Keap1 TR-FRET (time-resolved fluorescence resonance energy transfer) low protein assay, full length Nrf2 protein and full length Keap1 protein (Keap1 exists a dimer) are used. The assay detects a compound's ability to displace the binding of Keap1 FlagHis with biotinylated Avi-Nrf2 protein. Biotin-Nrf2 binds to streptavidin-europium (a component of the detection mix) and Keap1 FlagHis is recognized by anti-Flag APC (allophycocyanin) antibody (also a component of the detection mix). If binding occurs between the two proteins, there will be an energy transfer from the Eu+3 (donor) at 615 nm to the APC (acceptor) at 665 nm. A potential Nrf2 inhibitor will cause a reduction in the TR-FRET signal by interfering with the binding of Keap1 to Nrf2.

Ten nanoliters of 100× compound dose response curves (serial 3-fold dilutions) in DMSO are stamped using an Echo liquid handling system (Labcyte) into 384-well, low volume, black assay plates (Greiner, #784076), with DMSO in columns 6 and 18. An additional 90 nl DMSO is added to each well, to bring the total volume to 100 nl per well. The top concentration of compound is located in columns 1 and 13, with the serial dilutions going across the row. All reagents are diluted in assay buffer (50 mM Tris, pH 8.0, 5 mM MgCl2, 100 mM NaCl, 0.005% BSA, 1 mM DTT, and 2 mM CHAPS. The BSA, DTT, and CHAPS are added to the assay buffer on the day of assay. Using a Multidrop Combi (Thermo Electron Corporation) equipped with a metal tip dispenser, 5 ul of 1.25 nM Keap1 FlagHis protein is added to all wells of the compound plate, with the exception of the wells in column 18. Wells in column 18 receive 5 ul of assay buffer instead. Plates are centrifuged at 500 rpm for 1 minute, covered with a plate lid, and incubated at 37° C. for 2.25 hours. Plates are then removed from the incubator and allowed to cool to RT for 15 minutes. Five microliters of 2.5 nM biotin-Nrf2 protein is then added to all wells of the plates and the plates are spun at 500 rpm for 1 minute, followed by incubating at 4° C. for 1.25 hours. The plates are then allowed to warm to RT for 15 minutes, followed by the addition of 10 ul of detection mix (1 nM Streptavidin Eu+ W1024 and 5 ug/ml mouse anti-DYKDDDDK IgG conjugated to SureLight APC antibody; both from Columbia Biosciences) to all wells. Plates are spun at 500 rpm for 1 minute, incubated for 1 hour at RT, and read on an Envision plate reader using a 320 nm excitation filter and 615 nm and 665 nm emission filters. Compound response (% inhibition) and potency (plC50) are calculated based on the ratio of the two emissions (665 nm/615 nm) and then the transformed data is normalized against controls in the assay (control 1=1% DMSO in the presence of Nrf2 and Keap1 protein and control 2=1% DMSO in the presence of only the Nrf2 protein). Data analysis is handled using the software package Abase XE (Surrey, United Kingdom). The % inhibition values are calculated from the ratio (transformed) data by the equation:

100-(100*(compound response-average control 2)/(average control 1-average control2)).
For calculation of plC50s, Abase XE uses a four parameter equation.

### Methods of Use

The compounds of the invention are Nrf2 regulators, and are useful in the treatment or prevention of respiratory disorders, including COPD, asthma, fibrosis, lung infection, diabetic nephropathy/chronic kidney disease, autoimmune diseases (e.g., multiple sclerosis and inflammatory bowel disease), eye diseases (e.g., AMD, Fuchs, and uveitis), cardiovascular diseases, Nonalcoholic steatohepatitis (NASH), Parkinson's, Alzheimer's, psoriasis, acute kidney injury, topical effects of radiation, and kidney transplant.

Accordingly, in another aspect the invention is directed to methods of treating such conditions.

The methods of treatment of the invention comprise administering a safe and effective amount of a compound according to Formula I or a pharmaceutically-acceptable salt thereof to a patient in need thereof.

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” in reference to a compound of the invention or other pharmaceutically-active agent means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human or other animal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical dosages for oral administration range from 1 mg to 1000 mg per person per day. Preferred dosages are 1-500 mg once daily, more preferred is 1-100 mg per person per day. IV dosages range form 0.1-000 mg/day, preferred is 0.1-500 mg/day, and more preferred is 0.1-100 mg/day. Inhaled daily dosages range from 10 ug-10 mg/day, with preferred 10 ug-2 mg/day, and more preferred 50 uug-500 ug/day.

Additionally, the compounds of the invention may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleaved in vivo. Such modifications, which include the preparation of phosphates, amides, ethers, esters, thioesters, carbonates, and carbamates, are well known to those skilled in the art.

### Compositions

The compounds of the invention will normally, but not necessarily, be formulated into pharmaceutical compositions prior to administration to a patient. Accordingly, in another aspect the invention is directed to pharmaceutical compositions comprising a compound of the invention and one or more pharmaceutically-acceptable excipient.

The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 1 mg to 1000 mg.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as dry powders, aerosols, suspensions, and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

In another aspect, the invention is directed to a dosage form adapted for administration to a patient parenterally including subcutaneous, intramuscular, intravenous or intradermal. Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In another aspect, the invention is directed to a dosage form adapted for administration to a patient by inhalation. For example, the compound of the invention may be inhaled into the lungs as a dry powder, an aerosol, a suspension, or a solution.

Dry powder compositions for delivery to the lung by inhalation typically comprise a compound of the invention as a finely divided powder together with one or more pharmaceutically acceptable excipients as finely divided powders. In one embodiment, the invention is directed to a dosage form adapted for administration to a patient by inhalation as a dry powder. In a further embodiment, the invention is directed to a dosage form adapted for administration to a patient by inhalation via a nebulizer. Pharmaceutically acceptable excipients particularly suited for use in dry powders are known to those skilled in the art and include lactose, starch, mannitol, and mono-, di-, and polysaccharides. The finely divided powder may be prepared by, for example, micronization and milling. Generally, the size-reduced (e.g., micronized) compound can be defined by a D50 value of about 1 to about 10 microns (for example as measured using laser diffraction).

The dry powder compositions for use in accordance with the present invention may be administered via inhalation devices. As an example, such devices can encompass capsules and cartridges of for example gelatin, or blisters of, for example, laminated aluminum foil. Blister packs may be for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 ug-10 mg of the compound of Formula (I) or a pharmaceutically acceptable salt thereof. In various embodiments, each capsule, cartridge or blister may contain doses of composition according to the teachings presented herein. Examples of inhalation devices can include those intended for unit dose or multi-dose delivery of composition, including all of the devices set forth herein.

As an example, in the case of multi-dose delivery, the formulation can be pre-metered (e.g., as in Diskus®, see GB2242134, U.S. Pat. Nos. 6,032,666, 5,860,419, 5,873,360, 5,590,645, 6,378,519 and 6,536,427 or Diskhaler, see GB 2178965, 2129691 and 2169265, U.S. Pat. Nos. 4,778,054, 4,811,731, 5,035,237) or metered in use (e.g. as in Turbuhaler, see EP 69715, or in the devices described in U.S. Pat. No 6,321,747). An example of a unit-dose device is Rotahaler (see GB 2064336). In one embodiment, the Diskus® inhalation device comprises an elongate strip formed from a base sheet having a plurality of recesses spaced along its length and a lid sheet peelably sealed thereto to define a plurality of containers, each container having therein an inhalable formulation containing the compound optionally with other excipients and additive taught herein. The peelable seal is an engineered seal, and in one embodiment the engineered seal is a hermetic seal. Preferably, the strip is sufficiently flexible to be wound into a roll. The lid sheet and base sheet will preferably have leading end portions which are not sealed to one another and at least one of the leading end portions is constructed to be attached to a winding means. Also, preferably the engineered seal between the base and lid sheets extends over their whole width. The lid sheet may preferably be peeled from the base sheet in a longitudinal direction from a first end of the base sheet.

A dry powder composition may also be presented in an inhalation device which permits separate containment of two different components of the composition. Thus, for example, these components are administrable simultaneously but are stored separately, e.g. in separate pharmaceutical compositions, for example as described in WO 03/061743 A1 WO 2007/012871 A1 and/or WO2007/068896. In one embodiment an inhalation device permitting separate containment of components is an inhaler device having two peelable blister strips, each strip containing pre-metered doses in blister pockets arranged along its length, e.g., multiple containers within each blister strip. Said device has an internal indexing mechanism which, each time the device is actuated, peels opens a pocket of each strip and positions the blisters so that each newly exposed dose of each strip is adjacent to the manifold which communicates with the mouthpiece of the device. When the patient inhales at the mouthpiece, each dose is simultaneously drawn out of its associated pocket into the manifold and entrained via the mouthpiece into the patient's respiratory tract. A further device that permits separate containment of different components is DUOHALER™ of Innovata. In addition, various structures of inhalation devices provide for the sequential or separate delivery of the pharmaceutical composition(s) from the device, in addition to simultaneous delivery.

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Aerosols may be formed by suspending or dissolving a compound of the invention in a liquefied propellant. Suitable propellants include halocarbons, hydrocarbons, and other liquefied gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of the invention will typically be administered to a patient via a metered dose inhaler (MDI). Such devices are known to those skilled in the art.

The aerosol may contain additional pharmaceutically acceptable excipients typically used with multiple dose inhalers such as surfactants, lubricants, cosolvents and other excipients to improve the physical stability of the formulation, to improve valve performance, to improve solubility, or to improve taste.

Suspensions and solutions comprising a compound of the invention may also be administered to a patient via a nebulizer. The solvent or suspension agent utilized for nebulization may be any pharmaceutically acceptable liquid such as water, aqueous saline, alcohols or glycols, e.g., ethanol, isopropyl alcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically acceptable excipients may be added to the suspension or solution. The compound of the invention may be stabilized by the addition of an inorganic acid, e.g., hydrochloric acid, nitric acid, sulfuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of the invention. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

The compounds of formula (I) and pharmaceutically acceptable salts thereof may be used in combination with one or more other agents which may be useful in the prevention or treatment of allergic disease, inflammatory disease, autoimmune disease, for example; antigen immunotherapy, anti-histamines, corticosteroids, (eg fluticasone propionate, fluticasone furoate, beclomethasone dipropionate, budesonide, ciclesonide, mometasone furoate, triamcinolone, flunisolide), NSAIDs, leukotriene modulators (e.g. montelukast, zafirlukast, pranlukast), iNOS inhibitors, tryptase inhibitors, IKK2 inhibitors, p38 inhibitors, Syk inhibitors, protease inhibitors such as elastase inhibitors, integrin antagonists (e.g., beta-2 integrin antagonists), adenosine A2a agonists, mediator release inhibitors such as sodium chromoglycate, 5-lipoxygenase inhibitors (zyflo), DP1 antagonists, DP2 antagonists, PI3K delta inhibitors, ITK inhibitors, LP (lysophosphatidic) inhibitors or FLAP (5-lipoxygenase activating protein) inhibitors (e.g. sodium 3-(3-(tert-butylthio)-1-(4-(6-ethoxypyridin-3-yl)benzyl)-5-((5-methylpyridin-2-yl)methoxy)-1H-indol-2-yl)-2,2-dimethylpropanoate), bronchodilators (e.g., muscarinic antagonists, beta-2 agonists), methotrexate, and similar agents; monoclonal antibody therapy such as anti-IgE, anti-TNF, anti-IL-5, anti-IL-6, anti-IL-12, anti-IL-1 and similar agents; cytokine receptor therapies e.g. etanercept and similar agents; antigen non-specific immunotherapies (e.g. interferon or other cytokines/chemokines, chemokine receptor modulators such as CCR3, CCR4 or CXCR2 antagonists, other cytokine/chemokine agonists or antagonists, TLR agonists and similar agents).

The compounds may also be used in combination with agents for aiding transplantation including Cyclosporines, Tacrolimus, Mycophenolate mofetil, Prednisone, Azathioprine , Sirolimus, Daclizumab, Basiliximab, or OKT3.

They may also be used in combination with agents for Diabetes: metformin (biguanides), meglitinides, sulfonylureas, DPP-4 inhibitors, Thiazolidinediones, Alpha-glucosidase inhibitors, Amylin mimetics, Incretin mimetics, insulin.

The compounds may be used in combination with antihypertensives such as diuretics, ACE inhibitors, ARBS, calcium channel blockers, and beta blockers.

One embodiment of the invention encompasses combinations comprising one or two other therapeutic agents. It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimize the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical formulation and thus pharmaceutical formulations comprising a combination as defined above together with a pharmaceutically acceptable diluent or carrier represent a further aspect of the invention.

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilled in the art.

The invention thus provides, in a further aspect, a pharmaceutical composition comprising a combination of a compound of the invention together with another therapeutically active agent.

### EXAMPLES

The invention will now be described by reference to the following examples which are merely illustrative and are not to be construed as a limitation of the scope of the present invention. All temperatures are given in degrees Celsius, all solvents are highest available purity and all reactions run under anhydrous conditions in an argon (Ar) or nitrogen (N₂) atmosphere where necessary.

Analtech Silica Gel GF and E. Merck Silica Gel 60 F-254 thin layer plates were used for thin layer chromatography. Both flash and gravity chromatography were carried out on silica gel 230-400, 100-200 & 60-120 Cilicant Brand. The CombiFlash® system used for purification in this application was purchased from Isco, Inc. CombiFlash® purification was carried out using prepacked silica gel columns, a detector with UV wavelength at 254 nm and a variety of solvents or solvent combinations.

Preparative HPLC was performed using a Gilson or Waters Preparative System with variable wavelength UV detection or an Agilent Mass Directed AutoPrep (MDAP) system or Shimadzu PREP LC 20AP with both mass and variable wavelength UV detection. A variety of reverse phase columns, e.g., Luna C18(2), SunFire C18, XBridge C18, Atlantics T3, Kromasil C18, Xbridge Phenyl-Hexyl columns were used in the purification with the choice of column support dependent upon the conditions used in the purification. The compounds are eluted using a gradient of CH₃CN or methanol and water. Neutral conditions used an CH₃CN and water gradient with no additional modifier, acidic conditions used an acid modifier, usually 0.1% TFA or 0.1% formic acid and basic conditions used a basic modifier, usually 0.1% NH₄OH (added to the water) or 10 mM ammonium bicarbonate (added to the water), or 0.05% NH₄HCO₃ (added to water).

Analytical HPLC was run using an Agilent system or Waters Alliance HPLC with 2996 PDA detector, Waters Acquity UPLC-MS or Agilent Infinity 1290 with PDA or conducted on a Sunfire C18 column, alternative on XSELECT CSH C18 column using reverse phase chromatography with a CH₃CN and water gradient with 0.1% formic acid modifier (added to each solvent) and basic conditions used a basic modifier, usually 5 mM ammonium bicarbonate or 10 mM ammonium bicarbonate in water adjusted pH to 10 with ammonia solution. The compound was analyzed by LCMS using a Shimadzu LC system with UV 214 nm wavelength detection and H₂O—CH₃CN gradient elution (4-95% over 1.9 min.) acidified to 0.02% TFA. The reversed-phase column was a 2.1×20 mm Thermo Hypersil Gold C18 (1.9 u particles) at 50° C. The single quadrupole MS detector was either a Sciex 150EX or a Waters ZQ operated in positive-ion. Alternatively, LC-MS was determined using either a PE Sciex Single Quadrupole 150EX LC-MS, or Waters ZQ Single Quadrupole, Waters 3100 Single Quadrupole, Agilent 6130 SQD or Agilent 6120 Single Quadrupole LC-MS instruments. The compound is analyzed using a reverse phase column, e.g., Thermo Hypersil Gold C18 and/or Luna C18 eluted using a gradient of CH₃CN and water with a low percentage of an acid modifier such as 0.02% or 0.1% TFA.

Preparative Chiral SFC was performed using a Thar/Waters Preparative SFC System with single wavelength UV detection system. A variety of chiral SFC columns, e.g. Chiralpak IA, IC, AY, AD, IF, OJ were used in the purification. The compounds are eluted using supercritical fluid CO₂ and co-solvents, such as MeOH, EtOH, IPA, and combination of these solvent in different ratio based on the compound. Modifiers (0.1% to 0.4% of TFA, NH₄OH, DEA, TEA) can be used as needed. Normal phase chromatography is performed using the above mentioned chiral columns & pyridyl amide, ethyl pyridine achiral columns are used for chiral & achiral purifications respectively. Modifiers (0.1% of TFA, NH4OH, DEA) would be used as needed. K PREP Lab 100—YMC instruments are used in normal phase preparative scale purifications.

Analytical Chiral SFC was run using a Thar/Waters SFC system with variable wavelength UV detection. A variety of chiral SFC columns, e.g. Chiralpak IA, IB, IC, ID, IF, AY, AD, OD, C2, AS, OJ, CCL4 were used in the purification. The compounds are eluted using supercritical fluid CO₂ and co-solvents, such as MeOH, EtOH, IPA, and combination of these solvent in different ratio based on the compound selectivity. Modifiers (0.1% to 0.4% of TFA, NH₄OH, DEA, TEA) would be used as needed.

Celite® is a filter aid composed of acid-washed diatomaceous silica, and is a registered trademark of Manville Corp., Denver, Colorado. (solute® is a functionalized silica gel based sorbent, and is a registered trademark of Biotage AB Corp., Sweden.

Nuclear magnetic resonance spectra were recorded at 400 MHz using a Bruker AVANCE 400 or Brucker DPX400 spectrometer or Varian MR400 spectrometer. CDCl₃ is deuteriochloroform, DMSO-D₆ is hexadeuteriodimethylsulfoxide, and MeOD is tetradeuteriomethanol, CD₂Cl₂ is deuteriodichloromethane. Chemical shifts are reported in parts per million (δ) downfield from the internal standard tetramethylsilane (TMS) or calibrated to the residual proton signal in the NMR solvent (e.g., CHCl₃ in CDCl₃). Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz.

Heating of reaction mixtures with microwave irradiation was carried out on a Biotage Initiator® microwave reactor, typically employing the high absorbance setting.

Cartridges or columns containing polymer based functional groups (acid, base, metal chelators, etc) can be used as part of compound workup. The “amine” columns or cartridges are used to neutralize or basify acidic reaction mixtures or products. These include NH₂ Aminopropyl SPE-ed SPE Cartridges available from Applied Separations and diethylamino SPE cartridges available from United Chemical Technologies, Inc.

Table of Abbreviations

[Rh(cod)Cl]2 or [RhCl(cod)]2: di-μ-chlorido-bis[η2,η2-(cycloocta-1,5-
diene)rhodium
®T3P: 2,4,6-tripropyl-1,3,5,2,4,6-trioxatriphosphorinane 2,4,6-trioxide
° C.: degree Celsius
AcOH: acetic acid
ADDP: (E)-diazene-1,2-diylbis(piperidin-1-ylmethanone)
aq = aqueous
BINAP: 2,2′-bis(diphenylphosphino)-1,1′-binaphthalene
CDI: Carbonyl dimidazole
CH2Cl2: dichloromethane
CH3CN: acetonitrile
CH3CN: acetonitrile
CHCl3: chloroform
Cs2CO3: cesium carbonate
DBU: 1,8-diazabicyclo[5.4.0]undec-7-ene
DCE: dichloroethane
DCM: dichloromethane
DIPEA or DIEA: diisopropylethyl amine
DME: dimethyl ether
DMF: N,N-dimethylformamide
DMF-DMA or DMF-dimethyl acetal: N,N-dimethylformaide-dimethyl
acetal
DMSO: dimethyl sulfoxide
EDC: 1-ethyl-3-(3-dimethylaminopropyl)carbodiimide
Et2O: diethyl ether
Et3N: triethylamine
EtOAc: ethyl acetate
EtOH: ethanol
g: gram(s)
h: hour(s)
HATU: O-(7-azabenzotriazol-1-yl)-N,N,N′,N′-tetramethyluronium
hexafluorophosphate
HBTU: N,N,N′,N′-tetramethyl-O-(1H-benzotriazol-1-yl)uronium
hexafluorophosphate
HCl: hydrochloric acid
HOAt: 1-hydroxy-7-azabenzotriazole
HPLC: high performance liquid chromatography
IPA: isopropyl alcohol
K2CO3: potassium carbonate
KOAc: Potassium acetate
LAH: lithium aluminum hydride
LC: liquid chromatography
LC-MS: liquid chromatography-mass spectroscopy
LiBH4: lithium borohydride
LiHMDS: lithium hexamethyldisilazane
LiOH: lithium hydroxide
M: molar
MeCN: acetonitrile
Mel: methyl iodide
MeOH: methanol
mg: milligram(s)
MgCl2: magnesium chloride
MgSO4: magnesium sulfate
MHz: megahertz
min: minute(s)
mL: milliliter(s)
mmol: millimole(s)
MS: mass spectroscopy
N2: nitrogen gas
Na2CO3: sodium carbonate
Na2SO4: sodium sulfate
NaBH3CN or NaCNBH3: sodium cyanoborohydride
NaCl: sodium chloride
NaH: sodium hydride
NaHCO3: sodium bicarbonate
NaHMDS: sodium hexamethyldisilazane
NaHSO4: sodium bisulfate
NaOAc: sodium acetate
NaOH: sodium hydroxide
NBS: N-Bromosuccinimide
nBuLi: n-butyl lithium
NH4Cl: ammonium chloride
NMR: nuclear magnetic resonance
P(tBu)3: tri-t-butyl phosphine
Pd(PhP3)4: tetrakistriphenylphosphine palladium
Pd/C: pallidium on carbon
Pd2(dba)3: tris(dibenzylideneacetone)-dipalladium(0)
PdCl2(dppf) or Pd(dppf)Cl2: [1,1′-bis(diphenylphosphino)-ferrocene]
dichloropalladium(II)
Petrol: petroleum ether
PS-PPh3: polymer supported triphenylphosphine
PtO2: platinum(IV) oxide
RT: room temperature
T3P: 2,4,6-tripropyl-1,3,5,2,4,6-trioxatriphosphorinane-2,4,6-trioxide
solution
TEA: triethylamine
TFA: trifluoroacetic acid
TFFH: Tetrafluoroformamidinium hexafluorophosphate
THF: tetrahydrofuran
triflic anhydride: trifluoromethanesulfonic anhydride
TsOH: p-toluenesulfonic acid
wt %: weight percent

### Intermediate 1

### 3-(4-Chloro-3-hydroxymethyl-phenyl)-3-(1-methyl-1H-benzotriazol-5-yl)-propionic acid methyl ester

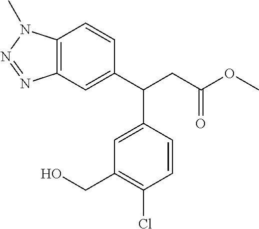 [SMILES: COC(=O)CC(c1ccc(Cl)c(CO)c1)c1ccc2c(c1)nnn2C]

A stirred mixture of (2-chloro-5-(4,4,5,5-tetramethyl-1,3,2-dioxaborolan-2-yl)phenyl)methanol (0.927 g, 3.45 mmol), (E)-3-(1-Methyl-1H-benzotriazol-5-yl)-acrylic acid methyl ester (0.500 g, 2.30 mmol), [RhCl(cod)]₂ (0.057 g, 0.12 mmol), triethylamine (0.349 g, 3.45 mmol), 1,4-dioxane (1.2 ml) and water (7.7 ml) was heated at 95° C. for 6 hours. Additional (2-chloro-5-(4,4,5,5-tetramethyl-1,3,2-dioxaborolan-2-yl)phenyl)methanol (0.5 eq) and [RhCl(cod)]₂ (0.050 g, 0.10 mmol) were added and the mixture heated again for 16 hours. The mixture was diluted with water and extracted with CHCl₃:IPA (3:1, ×3). The combined organic layers were washed successively with water and brine, dried over MgSO₄, filtered and concentrated to dryness. The residue was taken up into MeOH and the solution treated with 2 drops conc. HCl, then stirred under reflux for 4 hours. Concentration to dryness followed by purification by silica gel chromatography (100% DCM) gave the product as a white solid (0.560 g, 68%). LC-MS m/z 360/362(Cl) (M+H)⁺, 1.18 min (ret. time).

### Intermediate 2

### (E)-3-(1-Methyl-1H-benzotriazol-5-yl)-acrylic acid methyl ester

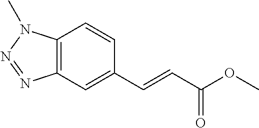 [SMILES: COC(=O)/C=C/c1ccc2c(c1)nnn2C]

A stirred solution of t-BuOK (2.10 g, 18.6 mmol) in THF (100 ml) at 0° C. under N₂ was treated with trimethyl phosphonoacetate (4.30 g, 23.3 mmol). After 30 mins, 1-methyl-1H-1,2,3-benzotriazole-5-carbaldehyde (2.50 g, 15.5 mmol) was added slowly in portions. After 1 hour, the mixture was treated with NH₄Cl (aq., sat.), diluted with water and then extracted with n-heptane (×3), resulting in a precipitate which was isolated by filtration. The combined organic layers were washed with water and brine, dried over MgSO₄, filtered and concentrated to dryness, giving a beige solid which was washed with toluene and dried under vacuum. This material was combined with the precipitate above to give the product as a beige solid (3.1 g, 92%). LC-MS m/z 218 (M+H)⁺ 1.12 min (ret. time).

### Intermediate 3

### 3-(3-Hydroxymethyl-4-methyl-phenyl)-3-(1-methyl-1H-benzotriazol-5-yl)-propionic acid methyl ester

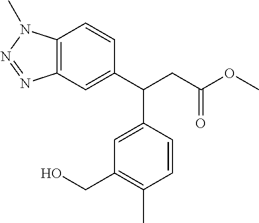 [SMILES: COC(=O)CC(c1ccc(C)c(CO)c1)c1ccc2c(c1)nnn2C]

A stirred mixture of (E)-3-(1-methyl-1H-benzotriazol-5-yl)-acrylic acid methyl ester (0.325 g, 1.50 mmol), 3-hydroxymethyl-4-methylbenzeneboronic acid (0.498 g, 3.0 mmol), [RhCl(cod)]₂ (0.037 g, 0.08 mmol), triethylamine (0.3 ml, 2.25 mmol), 1,4-dioxane (5 ml) and water (0.5 ml) was heated at 95° C. for 3 hours. After cooling to ambient temperature the mixture was diluted with water and extracted with EtOAc (×3). The combined organic layers were washed successively with water and brine, dried over MgSO₄, filtered and concentrated to dryness. Purification using silica gel chromatography (EtOAc/petrol gradient 30-80%) gave the product as a white solid (0.406 g, 80%). LC-MS m/z 340 (M+H)⁺ 1.16 min (ret. time).

### Intermediate 4

### 2-Methoxy-6-nitroaniline

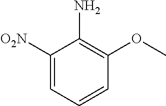

To a solution of 2-amino-3-nitrophenol (35 g, 227 mmol) in N,N-dimethylformamide (DMF) (400 mL), K₂CO₃ (37.7 g, 273 mmol) and iodomethane (17.04 mL, 273 mmol) were added at ambient temperature. The reaction mixture was stirred at ambient temperature for 16 h. Then it was poured into water. The resulting precipitate was collected by filtration and the solid was washed with water to give 35 g (89%) of the title compound. LC-MS m/z 168.9 (M+H)⁺, 1.71 min (ret. time).

### Intermediate 5

### 4-Bromo-2-methoxy-6-nitroaniline

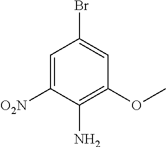

To a solution of 2-methoxy-6-nitroaniline (35 g, 208 mmol) in acetic acid (500 mL), sodium acetate (27.3 g, 333 mmol) and bromine (11.80 mL, 229 mmol) were added. Then the reaction mixture was stirred at ambient temperature for 20 minutes. The resulting precipitate was filtered and washed with water and dried in-vacuum pump to give 50 g (95%) of the title compound. LC-MS m/z 248.9 (M+H+2)⁺, 1.78 min (ret. time).

### Intermediate 6

### 4-Bromo-2-methoxy-N-methyl-6-nitroaniline

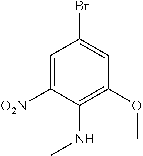

To a solution of 4-bromo-2-methoxy-6-nitroaniline (50 g, 202 mmol) in N,N-dimethylformamide (DMF) (400 mL) at 0° C., NaH (5.83 g, 243 mmol) was added. After 30 minutes, iodomethane (13.92 mL, 223 mmol) was added and the reaction mixture was stirred 30 minutes further. Water (1000 mL) was added. The red precipitate was collected by filtration and washed with water, dried to give 50 g (71.8%) of the title compound. LC-MS m/z 263.0 (M+H+2)⁺, 1.86 min (ret. time).

### Intermediate 7

### 4-Bromo-6-methoxy-N1-methylbenzene-1,2-diamine

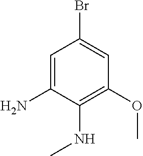

To 4-bromo-2-methoxy-N-methyl-6-nitroaniline (25 g, 96 mmol) in acetic acid (300 mL), zinc (18.78 g, 287 mmol) was added in small portions. Then the reaction mixture was stirred at ambient temperature for 10 h. The reaction mixture was filtered through celite and the solid was washed copiously with EtOAc. The combined solutions were concentrated to give 20 g (27.6%) of the title compound. LC-MS m/z 233.0 (M+H+2)⁺, 1.25 min (ret. time).

### Intermediate 8

### 5-Bromo-7-methoxy-1-methyl-1H-benzo[d][1,2,3]triazole

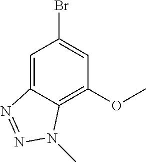

To 4-bromo-6-methoxy-N1-methylbenzene-1,2-diamine (40 g, 173 mmol) in 100 mL of 10% H₂SO₄ at 0° C., sodium nitrite (16.72 g, 242 mmol) was added in small portions over a 20 minute period. After the reaction mixture was stirred for 30 minutes further, 200 mL of water was added. The resulting precipitate was collected by filtration, washed with water and dried. The mother liquid was left to stand 16 h and a second batch of precipitate formed, which was collected as before. The combined solids were columned in EtOAc to remove inorganic salts, to give 15 g (35.8%) of the title compound. LC-MS m/z 243.0 (M+H+2)⁺, 1.68 min (ret. time).

### Intermediate 9

### (E)-Ethyl 3-(7-methoxy-1-methyl-1H-benzo[d][1,2,3]triazol-5-yl)acrylate

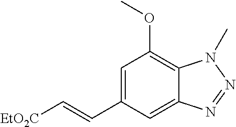 [SMILES: CCOC(=O)/C=C/c1cc(OC)c2c(c1)nnn2C]

To a solution of 5-bromo-7-methoxy-1-methyl-1H-benzo[d][1,2,3]triazole (10 g, 41.3 mmol) in dry N,N-dimethylformamide (DMF) (10 mL), ethyl acrylate (20.68 g, 207 mmol), DIPEA (18.04 mL, 103 mmol), and tri-o-tolylphosphine (2.51 g, 8.26 mmol) were added, followed by Pd(OAc)₂ (0.927 g, 4.13 mmol). The reaction was heated to 95° C. under a nitrogen atmosphere for 4 h. The reaction mixture was diluted with water and extracted with EtOAc (×3). Combined organic fractions were dried over MgSO₄ and concentrated. The residue was purified by silica gel chromatography (10-50% EtOAc/Petrol) to give 9.2 g (83%) of the title compound. LC-MS m/z 262.1 (M+H)⁺, 1.70 min (ret. time).

### Intermediate 10

### 3-(3-Hydroxymethyl-4-methyl-phenyl)-3-(7-methoxy-1-methyl-1H-benzotriazol-5-yl)-propionic acid ethyl ester

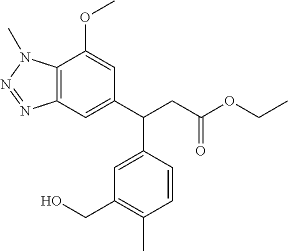 [SMILES: CCOC(=O)CC(c1ccc(C)c(CO)c1)c1cc(OC)c2c(c1)nnn2C]

Prepared by a similar procedure to 3-(3-Hydroxymethyl-4-methyl-phenyl)-3-(1-methyl-1H-benzotriazol-5-yl)-propionic acid methyl ester, using E)-Ethyl 3-(7-methoxy-1-methyl-1H-benzo[d][1,2,3]triazol-5-yl)acrylate (0.261 g, 1 mmol), 3-hydroxymethyl-4-methylbenzeneboronic acid (0.249 g, 1.5 mmol), [RhCl(cod)]₂ (0.025 g, 0.05 mmol), triethylamine (0.20 ml, 1.5 mmol), 1,4-dioxane (2 ml) and water (0.2 ml), followed by silica chromatography (EtOAc/petrol gradient 0-60%) to give a white solid (0.143 g, 37%). LC-MS m/z 384 (M+H)⁺, 1.26 min (ret. time).

### 3-Methyl-2-nitrobenzamide

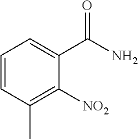

To a solution of 3-methyl-2-nitrobenzoic acid (100 g, 552 mmol) in Dichloromethane (DCM) (1000 mL), oxalyl chloride (72.5 mL, 828 mmol) was added at 25° C. The reaction mixture was stirred at ambient temperature for 1 h. The solvent was removed under reduced pressure. The residue was dissolved in CH₂Cl₂ (100 mL). The solvent was added to ammonium hydroxide (1000 mL, 7704 mmol) at ambient temperature and was stirred for 30 minutes. Then the reaction mixture was extracted with ethyl acetate (3×500 mL). The combined organic layer was dried over MgSO4 and concentrated to give 67 g (60.6%) of the title compound. LC-MS m/z 181.1 (M+H)⁺, 1.40 min (ret. time).

### 3-Methyl-2-nitroaniline

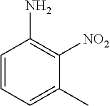

To a mixture of NaOH (2.220 g, 55.5 mmol) in water (12 mL), Br₂ (0.322 mL, 6.26 mmol) was added at 0° C. Then 3-methyl-2-nitrobenzamide (1 g, 5.55 mmol) was added in one portion, and the mixture is warmed slowly in a water bath. The material soon darkens in color, and at 50-55° C. (internal temperature) oil droplets begin to separate. The temperature is raised gradually to 70° C. and maintained at this point for one hour. A solution of 0.7 g of sodium hydroxide in 4 cc. of water was added slowly and the temperature is increased to 80° C. for an additional hour. The reaction was cooled to ambient temperature and extracted with ethyl acetate (3×50 mL). The combined organic layer was dried and concentrated to give 0.7 g (90%) of the title compound. LC-MS m/z 153.1 (M+H)⁺, 1.65 min (ret. time).

### 4-bromo-3-methyl-2-nitroaniline

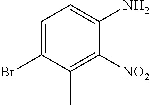

A mixture of NBS (51.5 g, 289 mmol), 3-methyl-2-nitroaniline (44 g, 289 mmol) and acetic acid (450 mL) was stirred at 110° C. for 1 h. The mixture was cooled to ambient temperature and poured into water (100 mL). The solid was collected to give 55 g (78%) of the title compound. LC-MS m/z 230.9 (M+H)⁺, 1.78 min (ret. time).

### 4-Bromo-N¹,3-dimethyl-2-nitroaniline

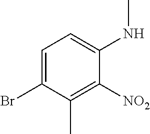

To a solution of 4-bromo-3-methyl-2-nitroaniline (20 g, 87 mmol) in N,N-dimethylformamide (200 mL), NaH (3.81 g, 95 mmol) was added at 25° C. The reaction mixture was stirred at 25° C. for 30 minutes. Then iodomethane (12.90 g, 91 mmol) was added. The reaction mixture was stirred for 12 h. The reaction mixture was poured into water and the solid was collected to give 18 g (59.4%) of the title compound. LC-MS m/z 247.0 (M+H+2)⁺, 1.90 min (ret. time).

### 4-bromo-N¹,3-dimethylbenzene-1,2-diamine

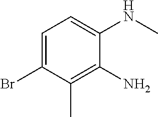

To a solution of 4-bromo-N,3-dimethyl-2-nitroaniline (65 g, 265 mmol) in ethanol (600 mL) and water (300 mL) was added ammonium chloride (142 g, 2652 mmol) followed by addition of iron (59.2 g, 1061 mmol) at ambient temperature. Then the reaction mixture was stirred at 90° C. for 4 h. The reaction mixture was cooled to ambient temperature and filtered through a celite pad, washed with EtOAc (100 mL) and the filtrate was evaporated under vacuum. The residue was diluted with a NaHCO₃ solution (500 mL) and extracted with EtOAc (2×500 mL). The combined organic layers were washed with a brine solution (500 mL) and dried over Na₂SO₄, filtered and the solvent was evaporated under vacuum. The crude residue was purified through column chromatography by using EtOAc Hexane (3:7). The eluted fractions were evaporated under vacuum to afford 4-bromo-N1,3-dimethylbenzene-1,2-diamine (46 g, 61.7% yield). LC-MS m/z 216.95 (M+H+2)⁺, 2.54 min (ret. time).

### 5-bromo-1,4-dimethyl-1H-benzo[d][1,2,3]triazole

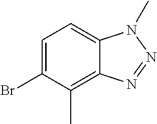

To 4-bromo-N¹,3-dimethylbenzene-1,2-diamine (30 g, 139 mmol) in 17 ml of 10% H₂SO₄ at 0° C., sodium nitrite (13.47 g, 195 mmol) was added in small portions over a 20 minute period. After the reaction mixture was stirred for 30 minutes further, 200 mL of water was added. The resulting precipitate was collected by filtration, washed with water and dried. The mother liquid was left to stand 16 h and a second batch of precipitate formed, which was collected as before. The combined solids were columned in EtOAc to remove inorganic salts to give 10 g (21.5%) of the title compound. LC-MS m/z 226.0 (M+H)⁺, 228.0 (M+H+2)⁺ 1.71 min (ret. time).

### (E)-ethyl 3-(1,4-dimethyl-1H-benzo[d][1,2,3]triazol-5-yl)acrylate

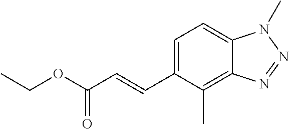 [SMILES: CCOC(=O)/C=C/c1ccc2c(nnn2C)c1C]

To a solution of 5-bromo-1,4-dimethyl-1H-benzo[d][1,2,3]triazole (10 g, 44.2 mmol) in DMF (20 mL), tri-o-tolylphosphine (2.69 g, 8.85 mmol), methyl acrylate (7.62 g, 88 mmol) and DIPEA (23.18 mL, 133 mmol) were added. Then Pd(OAc)₂ (0.993 g, 4.42 mmol) was added. The reaction mixture was stirred at 100° C. for 12 h. The mixture was poured into water and extracted with EtOAc (30 mL). The organic layer was dried and concentrated to get crude product. It was purified by silica gel chromatography column (petroleum ether: EtOAc=4:1) to give 8.2 g (76%) of the title compound. LC-MS m/z 246.1 (M+H)⁺, 1.68 (ret. time).

### Example 1

### 3-[3-({[(tert-Butoxy)carbonyl](methyl)amino}methyl)-4-chlorophenyl]-3-(1,4-dimethyl-1H-1,2,3-benzotriazol-5-yl)propanoic acid

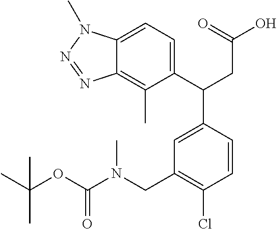 [SMILES: Cc1c(C(CC(=O)O)c2ccc(Cl)c(CN(C)C(=O)OC(C)(C)C)c2)ccc2c1nnn2C]

### Methanesulfonic acid 5-bromo-2-chloro-benzyl ester

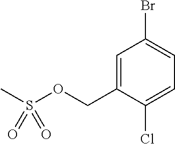

A stirred solution of 5-bromo-2-chlorobenzyl alcohol (0.886 g, 4.0 mmol) in DCM (40 ml) at 4° C. was treated with DIPEA (1.04 ml, 6.0 mmol) followed by, drop wise, MsCl (0.46 ml, 6.0 mmol). The mixture was warmed to ambient temperature. After 3 h, the mixture was diluted with water and extracted with DCM (3×). The combined organic phases were dried (MgSO₄), filtered and concentrated to dryness to give the product, used without further purification (0.976 g, 77%). LCMS MH⁺ 316/318/320, ambient temperature 1.32 min.

### (5-Bromo-2-chloro-benzyl)-methyl-amine

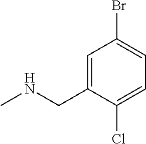

A stirred solution of methanesulfonic acid 5-bromo-2-chloro-benzyl ester (0.976 g, 3.26 mmol) in THF (20 ml) was treated with DIPEA (0.57 ml, 3.26 mmol) and MeNH₂ (2M in THF, 3.26 ml). After 15 h the mixture was diluted with 1M HCl and washed with EtOAc. The aqueous phase was brought to basic pH with Na₂CO₃ and extracted with EtOAc (3×). The combined organic phases were dried, filtered and concentrated to dryness to give the product, used without further purification (0.373 g, 48%). LCMS MH⁺ 234/236/238, ambient temperature 1.30 min.

### [2-Chloro-5-(4,4,5,5-tetramethyl-[1,3,2]dioxaborolan-2-yl)-benzyl]-methyl-carbamic acid tert-butyl ester

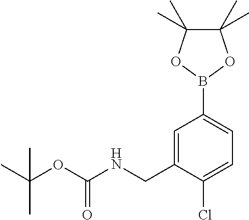 [SMILES: CC(C)(C)OC(=O)NCc1cc(B2OC(C)(C)C(C)(C)O2)ccc1Cl]

A stirred solution of (5-bromo-2-chloro-benzyl)-methyl-amine (0.373 g, 1.6 mmol) in THF (6 ml) was treated with BOC₂O (0.452 g, 2.1 mmol) and stirred for 15 h. The mixture was diluted with citric acid (1M aq.) and extracted with EtOAc (×3). The combined organic phases were dried (MgSO₄), filtered and concentrated to dryness. The residue was dissolved in dioxane (15 ml) and treated with bis(pinacolato)diboron (0.457 g, 1.8 mmol) and KOAc (0.314 g, 3.2 mmol). The resulting mixture was degassed with N₂, treated with PdCl₂dppf (0.058 g, 0.08 mmol), and stirred at 95° C. for 24 hours. The reaction was diluted with water and extracted with EtOAc (×2). The combined organic layers were washed with brine, dried over MgSO₄, filtered and concentrated to dryness. The residue was purified by silica gel chromatography (EtOAc/petrol gradient 0-40%) to give the product (0.240 g, 46%). LCMS MH⁺ 326/328, ambient temperature 1.69 min.

### 3-[3-({[(tert-Butoxy)carbonyl](methyl)amino}methyl)-4-chlorophenyl]-3-(1,4-dimethyl-1H-1,2,3-benzotriazol-5-yl)propanoic acid

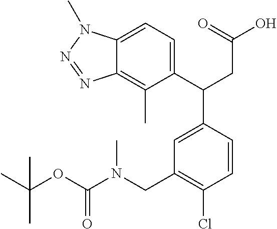 [SMILES: Cc1c(C(CC(=O)O)c2ccc(Cl)c(CN(C)C(=O)OC(C)(C)C)c2)ccc2c1nnn2C]

A stirred mixture of (E)-ethyl 3-(1,4-dimethyl-1H-benzo[d][1,2,3]triazol-5-yl)acrylate (0.054 g, 0.22 mmol), [2-chloro-5-(4,4,5,5-tetramethyl-[1,3,2]dioxaborolan-2-yl)-benzyl]-methyl-carbamic acid tert-butyl ester (0.100 g, 0.26 mmol), [RhCl(cod)]₂ (0.005 g, 0.01 mmol), triethylamine (0.045 ml, 0.33 mmol), 1,4-dioxane (2 ml) and water (0.2 ml) was degassed with N₂ and heated at 95° C. for 4 hours. After cooling to ambient temperature, LiOH (1M, 2 ml) was added and the mixture stirred at ambient temperature for 15 hours. The mixture was washed with DCM, acidified with HCl (1M, aq.) and extracted with DCM (×2). The combined organic layers were dried over MgSO₄, filtered and concentrated to dryness. Purification by preparative HPLC gave the product (0.005 g, 5%). LCMS MH⁺ 473, ambient temperature 1.11 min. ¹H NMR (400 MHz, Me-d₃-OD): 7.53 (2H, m), 7.31 (2H, m), 6.96 (1H, s), 5.04 (1H, t), 4.45 (2H, s), 4.29 (3H, s), 3.03-2.84 (2H, m), 2.76 (6H, m), 1.44-1.14 (9H, m).

### Example 2

### 3-[4-Chloro-3-({[(cyclopentyloxy)carbonyl](methyl)amino}methyl)phenyl]-3-(1-methyl-1H-1,2,3-benzotriazol-5-yl)propanoic acid

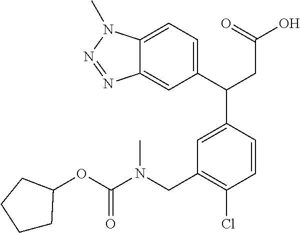 [SMILES: CN(Cc1cc(C(CC(=O)O)c2ccc3c(c2)nnn3C)ccc1Cl)C(=O)OC1CCCC1]

### 3-(4-Chloro-3-methylaminomethyl-phenyl)-3-(1-methyl-1H-benzotriazol-5-yl)-propionic acid methyl ester

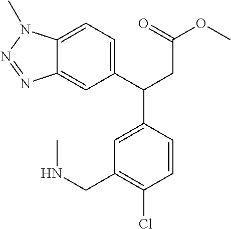 [SMILES: CNCc1cc(C(CC(=O)OC)c2ccc3c(c2)nnn3C)ccc1Cl]

A stirred solution of 3-(4-chloro-3-hydroxymethyl-phenyl)-3-(1-methyl-1H-benzotriazol-5-yl)-propionic acid methyl ester (1.00 g, 3.08 mmol) in dichloromethane (30 mL) at −78° C. under argon was treated with DIPEA (3.76 mL, 21.6 mmol) followed by, drop wise, methane-sulfonyl chloride (MsCl) (1.19 mL, 15.4 mmol). After 90 min the mixture was treated with methylamine (2M solution in THF, 86 mL, 172 mmol) and allowed to warm to ambient temperature. After a further 1 h the mixture was washed with aqueous NaCl/NaHCO₃ solution (4×). The organic phase was dried over Na₂SO₄, filtered and concentrated to dryness to give the product, used without further purification (0.772 g, 70%). LCMS MH⁺ 373/375, ambient temperature 1.21 min.

### 3-[4-Chloro-3-({[(cyclopentyloxy)carbonyl](methyl)amino}methyl)phenyl]-3-(1-methyl-1H-1,2,3-benzotriazol-5-yl)propanoic acid

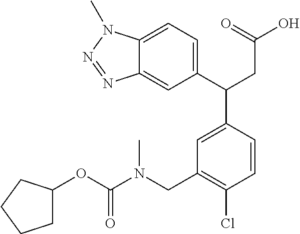 [SMILES: CN(Cc1cc(C(CC(=O)O)c2ccc3c(c2)nnn3C)ccc1Cl)C(=O)OC1CCCC1]

A stirred solution of 3-(4-chloro-3-methylaminomethyl-phenyl)-3-(1-methyl-1H-benzotriazol-5-yl)-propionic acid methyl ester (296 mg, 0.66 mmol) in anhydrous DCM (3.0 ml) at 4° C. under N₂ was treated with triethylamine (0.22 uL, 2.0 eq.) followed by, portionwise, 4-nitrophenyl chloroformate (0.17 g, 1.2 eq.). After 1 hour the reaction was diluted with water and extracted with DCM (3×). The combined organic layers were washed with brine and dried over MgSO₄, filtered and concentrated to dryness to give a pale yellow oil (350 mg). A portion of this material (120 mg) was taken up into THF (2 ml) and, with stirring at 4° C. under N₂, treated with cyclopentanol (0.035 g, 0.40 mmol) followed by NaH (0.011 g, 0.27 mmol). After 90 min. the mixture was diluted with MeOH (3 ml), treated with LiOH (1M aq., 2 ml), and allowed to warm to ambient temperature. After a further 2 hours the mixture was diluted with citric acid (5%, aq) and extracted with DCM (3×). The combined organic layers were washed with brine, dried over MgSO₄, filtered and concentrated to dryness to give a colourless oil (0.005 g, 8%). LCMS MH⁺ 471/473, ambient temperature 1.12 min. ¹H NMR (400 MHz, Me-d₃-OD): 7.90 (1H, s), 7.68 (1H, d), 7.52-7.41 (1H, m), 7.37 (1H, d), 7.34-7.26 (1H, m), 7.09 (1H, d), 4.98 (1H, d), 4.74 (1H, t), 4.53 (2H, d), 4.31 (3H, s), 3.17-3.06 (2H, m), 2.85 (3H, s), 1.89-1.30 (8H, m).

### Example 3

### 3-[3-({[(Butan-2-yloxy)carbonyl](methyl)amino}methyl)-4-chlorophenyl]-3-(1-methyl-1H-1,2,3-benzotriazol-5-yl)propanoic acid

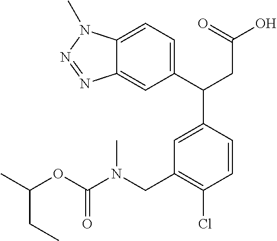 [SMILES: CCC(C)OC(=O)N(C)Cc1cc(C(CC(=O)O)c2ccc3c(c2)nnn3C)ccc1Cl]

Prepared using an analogous procedure to 3-[4-chloro-3-({[(cyclopentyloxy)carbonyl](methyl)amino}methyl)phenyl]-3-(1-methyl-1H-1,2,3-benzotriazol-5-yl)propanoic acid, using 2-butanol in place of cyclopentanol, and using preparative HPLC to give the product as a colourless oil (0.005 g, 8%). LCMS MH⁺ 459/461, ambient temperature 1.13 min. ¹H NMR (400 MHz, Me-d₃-OD): 7.89 (1H, s), 7.68 (1H, d), 7.45 (1H, s), 7.37 (1H, d), 7.30 (1H, s), 7.11 (1H, d), 4.76-4.69 (1H, m), 4.57 (3H, s), 4.31 (3H, s), 3.18-3.07 (2H, m), 2.86 (3H, 2*s), 1.65-0.61 (8H, m).

### Example 4

### 3-[3-({[(tert-Butoxy)carbonyl](methyl)amino}methyl)-4-methylphenyl]-3-(7-methoxy-1-methyl-1H-1,2,3-benzotriazol-5-yl)propanoic acid

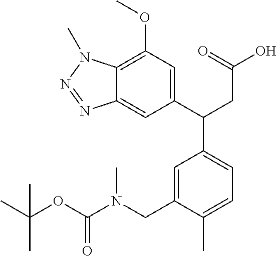 [SMILES: COc1cc(C(CC(=O)O)c2ccc(C)c(CN(C)C(=O)OC(C)(C)C)c2)cc2nnn(C)c12]

### 3-(3-Chloromethyl-4-methyl-phenyl)-3-(7-methoxy-1-methyl-1H-benzotriazol-5-yl)-propionic acid ethyl ester

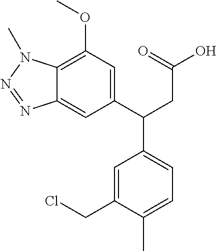 [SMILES: COc1cc(C(CC(=O)O)c2ccc(C)c(CCl)c2)cc2nnn(C)c12]

A stirred solution of 3-(3-hydroxymethyl-4-methyl-phenyl)-3-(7-methoxy-1-methyl-1H-benzotriazol-5-yl)-propionic acid ethyl ester (0.125 g, 0.33 mmol) in DCM (3 ml) at 4° C. was treated with DIPEA (0.085 ml, 0.49 mmol) followed by MsCl (0.033 ml, 0.49 mmol) and warmed to ambient temperature. After 2 h the mixture was diluted with water and extracted with EtOAc (×3). The combined organic layers were dried over MgSO₄, filtered and concentrated to dryness. Purification by silica chromatography gave a white solid (0.106 g, 80% yield). LCMS MH⁺ 402/404, ambient temperature 1.44 min.

### 3-[3-({[(tert-Butoxy)carbonyl](methyl)amino}methyl)-4-methylphenyl]-3-(7-methoxy-1-methyl-1H-1,2,3-benzotriazol-5-yl)propanoic acid

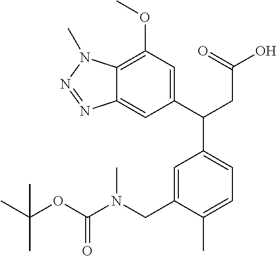 [SMILES: COc1cc(C(CC(=O)O)c2ccc(C)c(CN(C)C(=O)OC(C)(C)C)c2)cc2nnn(C)c12]

A stirred solution of 3-(3-chloromethyl-4-methyl-phenyl)-3-(7-methoxy-1-methyl-1H-benzotriazol-5-yl)-propionic acid ethyl ester (0.106 g, 0.26 mmol) in THF (2 ml) was treated with MeNH₂ (2M in THF, 1.3 ml) followed by Nal (0.004 g, 0.03 mmol). After 16 h the mixture was diluted with EtOAc, washed with water, dried over MgSO₄, filtered and concentrated to dryness. The residue was taken up into THF (3 ml) and, with stirring, treated with BOC₂O (0.059 g, 0.27 mmol). After 2 h the mixture was treated with LiOH (1M aq., 3 ml). After a further 16 hours the mixture was diluted with HCl (1M, aq.) and extracted with CHCl₃/IPA (3:1, ×2). The combined organic layers were dried over MgSO₄, filtered and concentrated to dryness to give the product (0.045 g, 37%). LCMS MH⁺ 469, ambient temperature 1.11 min. ¹H NMR (400 MHz, Me-d₃-OD): 7.52-7.31 (1H, m), 7.31-7.14 (1H, m), 7.11 (1H, d), 6.99 (1H, s), 6.86-6.67 (1H, m), 4.71-4.53 (1H, m), 4.40 (2H, s), 4.36 (3H, s), 3.94-3.79 (3H, m), 3.19-2.97 (2H, m), 2.96-2.61 (3H, m), 2.21 (3H, s), 1.60-1.20 (9H, m).

### Example 5

### 3-(4-Chloro-3-((((cyclohexyloxy)carbonyl)(methyl)amino)methyl)phenyl)-3-(1-methyl-1H-benzo[d][1,2,3]triazol-5-yl)propanoic acid

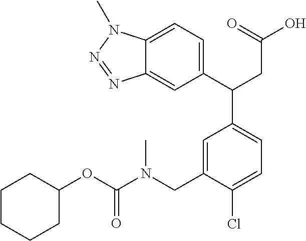 [SMILES: CN(Cc1cc(C(CC(=O)O)c2ccc3c(c2)nnn3C)ccc1Cl)C(=O)OC1CCCCC1]

### Methyl 3-(4-chloro-3-(hydroxymethyl)phenyl)-3-(1-methyl-1H-benzo[d][1,2,3]triazol-5-yl)propanoate

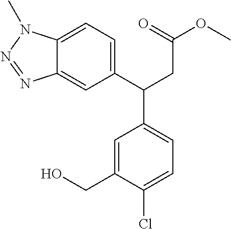 [SMILES: COC(=O)CC(c1ccc(Cl)c(CO)c1)c1ccc2c(c1)nnn2C]

To a suspension of (2-chloro-5-(4,4,5,5-tetramethyl-1,3,2-dioxaborolan-2-yl)phenyl)methanol (264 mg, 0.698 mmol), (E)-methyl 3-(1-methyl-1H-benzo[d][1,2,3]triazol-5-yl)acrylate (455 mg, 2.095 mmol) and chloro(1,5-cyclooctadiene)rhodium(I) dimer (34.4 mg, 0.070 mmol) in water (5 mL) and 1,4-dioxane (2 mL) at ambient temperature was added triethylamine (0.194 mL, 1.396 mmol) The resulting suspension was heated to 95° C. and left to stir for 2.5 hours. The reaction mixture was then cooled to 65° C. and left to stir at this temperature for a further 18 hours. The reaction mixture was cooled, diluted with water and extracted with ethyl acetate (4×). The organic phase washed with water (3×) and brine (1×), dried over magnesium sulfate, filtered and concentrated to give a brown solid. The crude product was then purified by flash chromatography to give the title compound (127 mg, 51%), LC-MS m/z 360.1 (M+H)⁺, 0.83 min (ret time). 100% purity.

### Methyl 3-(4-chloro-3-((methylamino)methyl)phenyl)-3-(1-methyl-1H-benzo[d][1,2,3] triazol-5-yl)propanoate

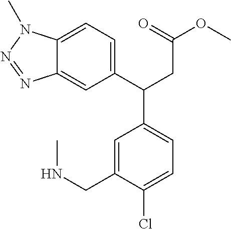 [SMILES: CNCc1cc(C(CC(=O)OC)c2ccc3c(c2)nnn3C)ccc1Cl]

To a solution of methyl 3-(4-chloro-3-(hydroxymethyl)phenyl)-3-(1-methyl-1H-benzo[d][1,2,3]triazol-5-yl)propanoate (135 mg, 0.375 mmol) and triethylamine (0.261 mL, 1.876 mmol) in dichloromethane (DCM) (6 mL) at −78° C. was added gradually mesyl chloride (0.073 mL, 0.938 mmol). The mixture was then stirred for 2 hr 15 mins at the same temperature under an argon atmosphere. Methanamine (1.126 mL, 2.251 mmol) was added and the mixture was left to return to ambient temperature while stirring for a further 2.5 h. After this time the reaction mixture was concentrated down and put into the freezer for 64.5 hours (over the weekend). Afterwards, the mixture was warmed to ambient temperature, taken up in DCM and washed with a 1:1 solution of NaCl and NaHCO₃ (4×). The organic phase was dried over sodium sulfate, filtered and concentrated down under reduced pressure to give a brown solid of the title compound (140 mg, 53%). LC-MS m/z 373.1 (M+H)⁺, 0.64 minutes (ret time). 53% purity.

### Methyl 3-(4-chloro-3-((((cyclohexyloxy)carbonyl)(methyl)amino)methyl)phenyl)-3-(1-methyl-1H-benzo[d][1,2,3]triazol-5-yl)propanoate

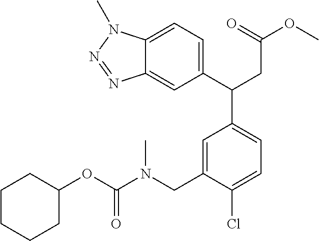 [SMILES: COC(=O)CC(c1ccc(Cl)c(CN(C)C(=O)OC2CCCCC2)c1)c1ccc2c(c1)nnn2C]

To a solution of methyl 3-(4-chloro-3-((methylamino)methyl)phenyl)-3-(1-methyl-1H-benzo[d][1,2,3]triazol-5-yl)propanoate (140 mg, 0.199 mmol) and triethylamine (0.042 mL, 0.299 mmol) in dichloromethane (DCM) (8 mL), in an ice bath, was added drop wise a solution of cyclohexyl carbonochloridate (0.029 mL, 0.199 mmol) in dichloromethane (DCM) (2 mL). The resulting solution was taken out of the ice bath and left to stir for 2.5 hours. The reaction was put in the fridge for 8 hours (overnight). Afterwards, the mixture was warmed to ambient temperature and further cyclohexyl carbonochloridate (0.01 mL, 0.070 mmol) in dichloromethane (DCM) (0.5 mL) was added. The solution was stirred for 4 hours. More cyclohexyl carbonochloridate (0.01 mL, 0.070 mmol) in dichloromethane (DCM) (0.5 mL) was added and stirred for 2 hours. Further cyclohexyl carbonochloridate (0.015 mL, 0.105 mmol) in dichloromethane (DCM) (0.5 mL) was added and this was left to stir for 18.5 hours. The reaction mixture was further diluted with DCM and washed with water (3×) and brine (1×). The organic layer was dried over magnesium sulfate, filtered and concentrated under reduced pressure resulting in a slightly brown oil. The crude product was purified by flash chromatography to give the title compound (61 mg, 61.4%), LC-MS m/z 499.1 (M+H)⁺, 1.23 minutes (ret time), 100% purity.

### 3-(4-Chloro-3-((((cyclohexyloxy)carbonyl)(methyl)amino)methyl)phenyl)-3-(1-methyl-1H-benzo[d][1,2,3]triazol-5-yl)propanoic acid

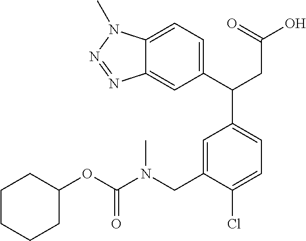 [SMILES: CN(Cc1cc(C(CC(=O)O)c2ccc3c(c2)nnn3C)ccc1Cl)C(=O)OC1CCCCC1]

To a solution of methyl 3-(4-chloro-3-((((cyclohexyloxy)carbonyl)(methyl)amino) methyl)phenyl)-3-(1-methyl-1H-benzo[d][1,2,3]triazol-5-yl)propanoate (61 mg, 0.122 mmol) in tetrahydrofuran (THF) (1 mL), methanol (1.000 mL) and water (1.000 mL) was added LiOH (14.64 mg, 0.611 mmol) and this was left to stir at ambient temperature for 1 hr 45 mins. After this time, 1 N HCl was added to the reaction mixture drop wise until the mixture was ˜pH 1. The acidic solution was diluted with EtOAc, washed with water (3×), dried over magnesium sulfate, filtered and the solvents removed under reduced pressure to afford a clear oil (54 mg, 91%), LC-MS m/z 485.2 (M+H)⁺, 1.1 min (ret time). 100% purity.

### Example 6

### 3-(3-(((tert-Butoxycarbonyl)(methyl)amino)methyl)-4-chlorophenyl)-3-(7-methoxy-1-methyl-1H-benzo[d][1,2,3]triazol-5-yl)propanoic acid

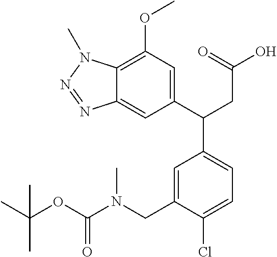 [SMILES: COc1cc(C(CC(=O)O)c2ccc(Cl)c(CN(C)C(=O)OC(C)(C)C)c2)cc2nnn(C)c12]

### (2-Chloro-5-(4,4,5,5-tetramethyl-1,3,2-dioxaborolan-2-yl)phenyl)methanol

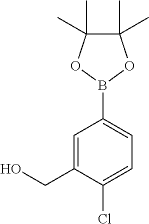 [SMILES: CC1(C)OB(c2ccc(Cl)c(CO)c2)OC1(C)C]

A suspension of (5-bromo-2-chlorophenyl)methanol (2.08 g, 9.39 mmol), potassium acetate (3.50 g, 35.7 mmol) and 4,4,4′,4′,5,5,5′,5′-octamethyl-2,2′-bi(1,3,2-dioxaborolane) (2.77 g, 10.89 mmol) degassed with a stream of nitrogen for ˜10 mins after which time bis(triphenylphosphine)palladium(II) chloride (0.396 g, 0.563 mmol) was added and the mixture was heated to 120° C. for 30 minutes in a microwave at high power. After this time, the suspension was cooled and filtered through celite using ethyl acetate to wash the celite. The dark colored solution was washed with water (4× gently), brine (1×), dried over magnesium sulfate, filtered and concentrated under reduced pressure. The crude product was purified by flash chromatography to give the title compound (2.76 g, 68.9%), LC-MS m/z 267.7 (M+H)⁺, 1.0 minutes (ret time), 63% purity.

### Ethyl 3-(4-chloro-3-(hydroxymethyl)phenyl)-3-(7-methoxy-1-methyl-1H-benzo[d][1,2,3]triazol-5-yl)propanoate

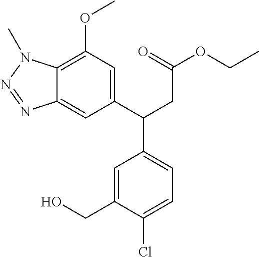 [SMILES: CCOC(=O)CC(c1ccc(Cl)c(CO)c1)c1cc(OC)c2c(c1)nnn2C]

To a suspension of (E)-ethyl 3-(7-methoxy-1-methyl-1H-benzo[d][1,2,3]triazol-5-yl)acrylate (1.41 g, 3.40 mmol), (2-chloro-5-(4,4,5,5-tetramethyl-1,3,2-dioxaborolan-2-yl)phenyl)methanol (1.370 g, 5.10 mmol), and triethylamine (0.707 ml, 5.10 mmol) in 1,4-dioxane (22.19 ml) and water (11.10 ml) at ambient temperature was added chloro(1,5-cyclooctadiene)rhodium(I) dimer (0.094 g, 0.191 mmol). The resulting suspension was heated at 95° C. for 30 minutes. The reaction mixture was diluted with water and extracted with EtOAc (3×). The combined organic phases were washed with water (3×), brine (1×), dried over magnesium sulfate and the solvent removed under reduced pressure. The crude product was purified by flash chromatography to give the title compound (1.02 g, 54.2%), LC-MS m/z 404.4 (M+H)⁺, 0.92 minutes (ret time), 73% purity.

### Ethyl 3-(4-chloro-3-((methylamino)methyl)phenyl)-3-(7-methoxy-1-methyl-1H-benzo[d][1,2,3]triazol-5-yl)propanoate

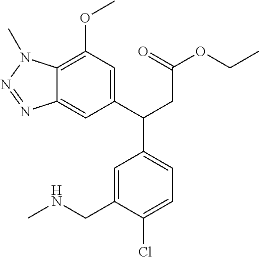 [SMILES: CCOC(=O)CC(c1ccc(Cl)c(CNC)c1)c1cc(OC)c2c(c1)nnn2C]

To a solution of ethyl 3-(4-chloro-3-(hydroxymethyl)phenyl)-3-(7-methoxy-1-methyl-1H-benzo[d][1,2,3]triazol-5-yl)propanoate (1.02 g, 1.844 mmol) and triethylamine (1.285 ml, 9.22 mmol) in dichloromethane (DCM) (18.02 ml) at −78° C. was added gradually mesyl chloride (0.359 ml, 4.61 mmol). The mixture was then stirred for 1 hour 20 minutes at the same temperature under a nitrogen atmosphere. Methanamine (5.53 ml, 11.06 mmol) was added and the mixture was left to return to ambient temperature while stirring for a further 2 and a half hours. After this time methanamine (5.53 ml, 11.06 mmol) was added to the mix and left to stir for a further 1 hr 30. The mixture was then taken up in DCM and washed with a 1:1 solution of NaCl and NaHCO3 (4×). The organic phase was dried over sodium sulfate, filtered and concentrated down under reduced pressure to give a brown solid. LC-MS m/z 417.2 (M+H)⁺, 0.72 minutes (ret time), 42% purity.

### Ethyl 3-(3-(((tert-Butoxycarbonyl)(methyl)amino)methyl)-4-chlorophenyl)-3-(7-methoxy-1-methyl-1H-benzo[d][1,2,3]triazol-5-yl)propanoate

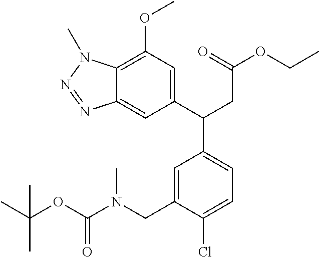 [SMILES: CCOC(=O)CC(c1ccc(Cl)c(CN(C)C(=O)OC(C)(C)C)c1)c1cc(OC)c2c(c1)nnn2C]

To a solution of ethyl 3-(4-chloro-3-((methylamino)methyl)phenyl)-3-(7-methoxy-1-methyl-1H-benzo[d][1,2,3]triazol-5-yl)propanoate (924 mg, 1.042 mmol), triethylamine (0.726 mL, 5.21 mmol) and boc-anhydride (0.508 mL, 2.188 mmol) in dichloromethane (DCM) (15 mL) was added DMAP (63.6 mg, 0.521 mmol) and the reaction left to stir at ambient temperature for 1 and a half hours. After this time the reaction was washed with 0.1 N HCl, the organic phase dried with magnesium sulfate, filtered and the solvent removed resulting in a white solid. The crude product was purified by flash chromatography to give the title compound (464 mg, 75%), LC-MS m/z 517.3 (M+H)⁺, 1.27 minutes (ret time), 87% purity.

### 3-(3-(((tert-Butoxycarbonyl)(methyl)amino)methyl)-4-chlorophenyl)-3-(7-methoxy-1-methyl-1H-enzo[d][1,2,3]triazol-5-yl)propanoic acid

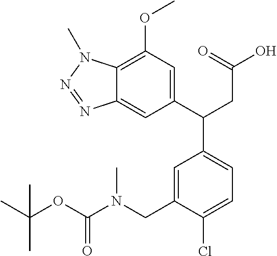 [SMILES: COc1cc(C(CC(=O)O)c2ccc(Cl)c(CN(C)C(=O)OC(C)(C)C)c2)cc2nnn(C)c12]

To a solution of ethyl 3-(3-(((tert-butoxycarbonyl)(methyl)amino)methyl)-4-chlorophenyl)-3-(7-methoxy-1-methyl-1H-benzo[d][1,2,3]triazol-5-yl)propanoate (0.1 g, 0.168 mmol) in 1,4-dioxane (2 ml) was added NaOH (0.168 ml, 0.337 mmol) and the mixture left to stir for 1 hour under a nitrogen atmosphere. The temperature was raised to 45° C. for 1 hour. Water (0.5 ml) and methanol (0.5 ml) were added to the reaction and the solution stirred for 45 mins. After this time, the solvent was removed. The residue was taken up in water and acidified to pH 4. The acidic mixture was extracted with EtOAc (3×), washed with brine (1×), dried over magnesium sulfate, filtered and the solvent removed to give a white solid (93 mg, 113%), LC-MS m/z 489.2 (M+H)⁺, 1.1 minutes (ret time), 100% purity.

### Example 7

### 3-(3-(2-(Dimethylamino)-2-oxoethoxy)-4-methylphenyl)-3-(1-methyl-1H-benzo[d][1,2,3]triazol-5-yl)propanoic acid

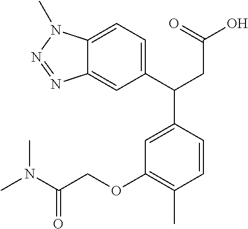 [SMILES: Cc1ccc(C(CC(=O)O)c2ccc3c(c2)nnn3C)cc1OCC(=O)N(C)C]

### (E)-Methyl 3-(1-methyl-1H-benzo[d][1,2,3]triazol-5-yl)acrylate

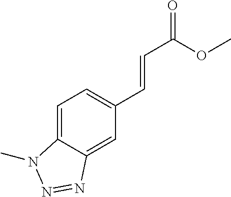

To a suspension of potassium tert-butoxide (2.65 g, 23.62 mmol) in THF (50 mL) at 0° C. was added methyl 2-(dimethoxyphosphoryl)acetate (4.0 mL, 24.82 mmol). After 30 min, 1-methyl-1H-benzo[d][1,2,3]triazole-5-carbaldehyde (2.57 g, 15.95 mmol) was added in small portions over 7 min. Then the suspension was stirred at 0° C. for 3 h and 45 min. The reaction was quenched with saturated ammonium chloride and diluted with water. The precipitate was filtered and dried to give the title compound (2.606 g, 75%) as grey solid. LC-MS: m/z 218.0 (M+H)⁺, 0.69 min (ret. time)

### 2-Methyl-5-(4,4,5,5-tetramethyl-1,3,2-dioxaborolan-2-yl)phenol

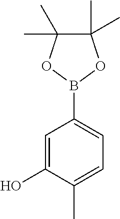 [SMILES: Cc1ccc(B2OC(C)(C)C(C)(C)O2)cc1O]

A suspension of 5-bromo-2-methylphenol (3 g, 16.04 mmol), 4,4,4′,4′,5,5,5′,5′-octamethyl-2,2′-bi(1,3,2-dioxaborolane) (4.72 g, 18.61 mmol), and potassium acetate (6.03 g, 61.4 mmol) was degassed with a stream of nitrogen for 10 minutes after which time (PPh₃)₂PdCl₂ (0.664 g, 0.946 mmol) was added. The resulting suspension was heated to 115° C. (bath temp, reflux) during which time the yellow colored suspension turned black for 2.5 h. The reaction mixture was filtered through celite using ethyl acetate to wash the celite. The dark colored solution was washed with water (2×), brine (1×). The organic layer was concentrated and purified with silica gel chromatography to give the title compound (1.91 g, 8.16 mmol, 50.9% yield) LC-MS: m/z 235.1 (M+H)⁺, 0.99 min (ret. time).

### Methyl 3-(3-hydroxy-4-methylphenyl)-3-(1-methyl-1H-benzo[d][1,2,3]triazol-5-yl)propanoate

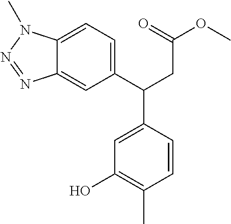 [SMILES: COC(=O)CC(c1ccc(C)c(O)c1)c1ccc2c(c1)nnn2C]

To a suspension of 2-methyl-5-(4,4,5,5-tetramethyl-1,3,2-dioxaborolan-2-yl)phenol (970 mg, 4.14 mmol), (E)-methyl 3-(1-methyl-1H-benzo[d][1,2,3]triazol-5-yl)acrylate (300 mg, 1.381 mmol), and [RhCl(cod)]₂ (68.1 mg, 0.138 mmol) in 1,4-dioxane (3 mL) and water (3 mL) at ambient temperature was added triethylamine (0.577 mL, 4.14 mmol). The resulting suspension was heated in a Biotage microwave at high absorption for 1 h at 150° C. The reaction mixture was passed through celite and washed with EtOAc. The filtrate was washed with water (2×), brine (1×). The organic layer was collected and concentrated to give the crude product. The crude product was purified by reverse-phase HPLC to give the title compound (330 mg, 1.014 mmol, 73.4% yield) as white solid. LC-MS: m/z 326.1 (M+H)⁺, 0.79 min (ret. time).

### Methyl 3-(3-(2-(dimethylamino)-2-oxoethoxy)-4-methylphenyl)-3-(1-methyl-1H-benzo[d][1,2,3]triazol-5-yl)propanoate

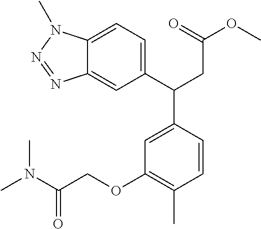

A mixture of methyl 3-(3-hydroxy-4-methylphenyl)-3-(1-methyl-1H-benzo[d][1,2,3]triazol-5-yl)propanoate (100 mg, 0.307 mmol), Cs₂CO₃ (200 mg, 0.615 mmol), 2-bromo-N,N-dimethylacetamide (128 mg, 0.768 mmol) and catalytic amount of Nal in acetonitrile (2 mL) was stirred at 60° C. for 24 h. It was filtered and the filtrate was concentrated. The crude product was purified by reverse-phase HPLC to give the title compound (50 mg, 0.122 mmol, 39.6% yield). LC-MS: m/z 411.1 (M+H)⁺, 0.83 min (ret. time).

### 3-(3-(2-(Dimethylamino)-2-oxoethoxy)-4-methylphenyl)-3-(1-methyl-1H-benzo[d][1,2,3]triazol-5-yl)propanoic acid

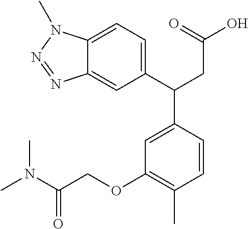 [SMILES: Cc1ccc(C(CC(=O)O)c2ccc3c(c2)nnn3C)cc1OCC(=O)N(C)C]

To a solution of methyl 3-(3-(2-(dimethylamino)-2-oxoethoxy)-4-methylphenyl)-3-(1-methyl-1H-benzo[d][1,2,3]triazol-5-yl)propanoate (50 mg, 0.122 mmol) in methanol (2 mL) at ambient temperature was added 2M LiOH (0.305 mL, 0.609 mmol). The mixture was stirred at ambient temperature for 2 h and 40 min. The pH was adjusted to ˜1 with 1N HCl. It was extracted with EtOAc (2×). The organic layer was dried over sodium sulfate, filtered and concentrated to give the crude product. The crude product was purified by reverse-phase HPLC (with 0.1% TFA condition) to give the title compound (35 mg, 0.088 mmol, 72.5% yield) as a white solid. LC-MS: m/z 397.1 (M+H)⁺, 0.72 min (ret. time) ¹H NMR (400 MHz, DMSO-d₆) δ=7.95 (s, 1H), 7.72 (d, J=8.5 Hz, 1H), 7.49 (d, J=8.5 Hz, 1H), 7.03 (d, J=7.3 Hz, 1H), 6.92-6.80 (m, 2H), 4.77 (s, 2H), 4.55 (t, J=7.5 Hz, 1H), 4.26 (s, 3H), 3.22-3.10 (m, 1H), 3.09-2.96 (m, 4H), 2.78 (s, 3H), 2.12 (s, 3H).

### Example 8

### 3-(1,4-Dimethyl-1H-benzo[d][1,2,3]triazol-5-yl)-3-(4-methyl-3-((methyl((4-nitrophenoxy)carbonyl)amino)methyl)phenyl)propanoic acid

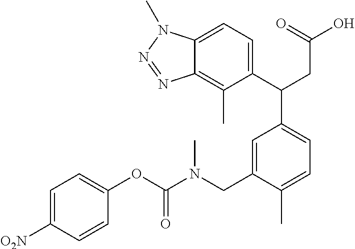 [SMILES: Cc1ccc(C(CC(=O)O)c2ccc3c(nnn3C)c2C)cc1CN(C)C(=O)Oc1ccc([N+](=O)[O-])cc1]

### Ethyl 3-(1,4-dimethyl-1H-benzo[d][1,2,3]triazol-5-yl)-3-(4-methyl-3-((methyl((4-nitrophenoxy)carbonyl)amino)methyl)phenyl)propanoate

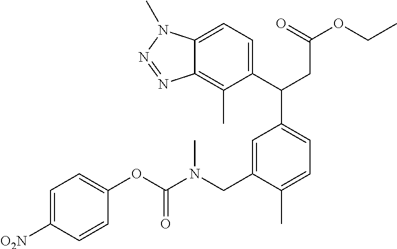 [SMILES: CCOC(=O)CC(c1ccc(C)c(CN(C)C(=O)Oc2ccc([N+](=O)[O-])cc2)c1)c1ccc2c(nnn2C)c1C]

To a solution of ethyl 3-(1,4-dimethyl-1H-benzo[d][1,2,3]triazol-5-yl)-3-(4-methyl-3-((methylamino)methyl)phenyl)propanoate (94 mg, 0.247 mmol) in pyridine (2 mL) was added 1-methoxy-2-methylpropan-2-yl(4-nitrophenyl)carbonate (86 mg, 0.321 mmol) and the mixture was left to stir at ambient temperature for 16 hours 30 min. After this time 1-methoxy-2-methylpropan-2-yl(4-nitrophenyl)carbonate (33.3 mg, 0.124 mmol) was added and the mixture stirred for a further 24.5 hours. The reaction was then diluted with EtOAc and washed (3×) with 10% sodium bisulfate solution. The organic phase was dried over sodium sulfate, filtered and the solvent removed under reduced pressure. The resulting residue was purified by flash chromatography resulting in a mixture of two compounds. This mixture was then purified by reverse phase HPLC (Sunfire 19×100 mm 5 u preparatory column) under neutral conditions eluting at 18 mL/min at a gradient of 20-80% acetonitrile/water to give the title compound (20 mg, 15%), LC-MS m/z 546.4 (M+H)⁺, 1.14 min (ret time), 100% purity.

### 3-(1,4-Dimethyl-1H-benzo[d][1,2,3]triazol-5-yl)-3-(4-methyl-3-((methyl((4-nitrophenoxy)carbonyl)amino)methyl)phenyl)propanoic acid

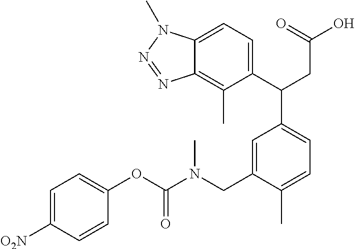 [SMILES: Cc1ccc(C(CC(=O)O)c2ccc3c(nnn3C)c2C)cc1CN(C)C(=O)Oc1ccc([N+](=O)[O-])cc1]

To a solution of ethyl 3-(1,4-dimethyl-1H-benzo[d][1,2,3]triazol-5-yl)-3-(4-methyl-3-((methyl((4-nitrophenoxy)carbonyl)amino)methyl)phenyl)propanoate (20 mg, 0.037 mmol) in tetrahydrofuran (THF) (0.5 mL) and water (0.250 mL) was added LiOH (4.39 mg, 0.183 mmol) and the mixture left to stir at ambient temperature for 17 hours. After this time, 1 N HCl was added to the reaction mixture drop wise until the mixture was at pH 1. The acidic solution was diluted with EtOAc, washed with water (3×), dried over magnesium sulfate, filtered and the solvents removed under reduced pressure to afford a white solid. The resulting residue was purified by reverse phase HPLC (Atlantics T3, 19×100 mm, 5 u prep column) under neutral eluting at 18 mL/min at a gradient of 35-65% acetonitrile/water to give the title compound (8 mg, 42%), LC-MS m/z 518.5 (M+H)⁺, 0.96 min (ret time), 100% purity.

### Example 9

### 3-(4-Chloro-3-((((cyclopentyloxy)carbonyl)(methyl)amino)methyl)phenyl)-3-(7-methoxy-1-methyl-1H-benzo[d][1,2,3]triazol-5-yl)propanoic acid

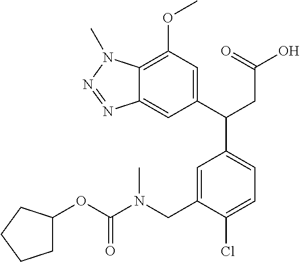 [SMILES: COc1cc(C(CC(=O)O)c2ccc(Cl)c(CN(C)C(=O)OC3CCCC3)c2)cc2nnn(C)c12]

### Ethyl 3-(4-chloro-3-((((cyclopentyloxy)carbonyl)(methyl)amino)methyl)phenyl)-3-(7-methoxy-1-methyl-1H-benzo[d][1,2,3]triazol-5-yl)propanoate

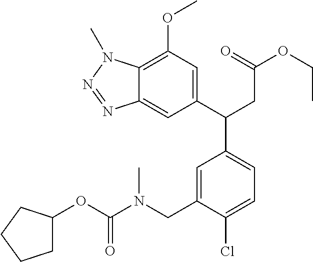 [SMILES: CCOC(=O)CC(c1ccc(Cl)c(CN(C)C(=O)OC2CCCC2)c1)c1cc(OC)c2c(c1)nnn2C]

To a solution of ethyl 3-(4-chloro-3-((methylamino)methyl)phenyl)-3-(7-methoxy-1-methyl-1H-benzo[d][1,2,3]triazol-5-yl)propanoate (150 mg, 0.331 mmol) and triethylamine (0.133 mL, 0.954 mmol) in dichloromethane (DCM) (8 mL), in an ice bath, was added dropwise a solution of cyclopentyl carbonochloridate (0.025 mL, 0.188 mmol). The resulting solution was taken out of the ice bath and left to stir for 19.5 h. After this time, was added additional triethylamine (0.115 mL, 0.828 mmol) and cyclopentyl carbonochloridate (0.044 mL, 0.331 mmol) and the reaction mixture left to stir for 1 h. The reaction mixture was further diluted with DCM and washed with water (3×) and brine (1×). The organic layer was dried over magnesium sulfate, filtered and concentrated to provide the title compound (186 mg, 101% yield). LC-MS m/z 529 (M+H)⁺, 1.28 min (ret. time).

### 3-(4-Chloro-3-((((cyclopentyloxy)carbonyl)(methyl)amino)methyl)phenyl)-3-(7-methoxy-1-methyl-1H-benzo[d][1,2,3]triazol-5-yl)propanoic acid

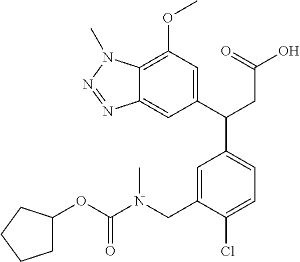 [SMILES: COc1cc(C(CC(=O)O)c2ccc(Cl)c(CN(C)C(=O)OC3CCCC3)c2)cc2nnn(C)c12]

To a solution of ethyl 3-(4-chloro-3-((((cyclopentyloxy)carbonyl)(methyl)amino)methyl)phenyl)-3-(7-methoxy-1-methyl-1H-benzo[d][1,2,3]triazol-5-yl)propanoate (184 mg, 0.330 mmol) in tetrahydrofuran (THF) (2 mL) and water (2.000 mL) was added LiOH (39.6 mg, 1.652 mmol) and stirred for 22 h. After this time, 1 N HCl was added to the reaction mixture dropwise until the mixture was at pH=1. The acidic solution was diluted with EtOAc, washed with water (3×), dried over magnesium sulfate, filtered and the solvents removed under reduced pressure to afford a yellow solid. The solid was purified by reverse phase preparative HPLC under neutral conditions to provide the title compound (186 mg, 101% yield). LC-MS m/z 529 (M+H)⁺, 1.28 min (ret. time).

### Example 10

### 3-(1,4-Dimethyl-1H-benzo[d][1,2,3]triazol-5-yl)-3-(3-((((((1-methoxy-2-methylpropan-2-yl)oxy)carbonyl)(methyl)amino)methyl)-4-methylphenyl)propanoic acid

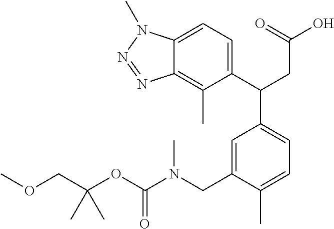 [SMILES: COCC(C)(C)OC(=O)N(C)Cc1cc(C(CC(=O)O)c2ccc3c(nnn3C)c2C)ccc1C]

### (E)-Ethyl 3-(1,4-dimethyl-1H-benzo[d][1,2,3]triazol-5-yl)acrylate

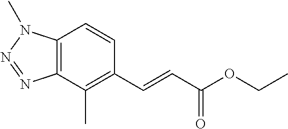 [SMILES: CCOC(=O)/C=C/c1ccc2c(nnn2C)c1C]

A solution of 5-bromo-1,4-dimethyl-1H-benzo[d][1,2,3]triazole (1100 mg, 4.87 mmol) in N,N-Dimethylformamide (DMF) (5 mL) at ambient temperature was added ethyl acrylate (3.11 mL, 29.2 mmol) and N-ethyl-N-isopropylpropan-2-amine (3.40 mL, 19.46 mmol), tri-o-tolylphosphine (444 mg, 1.460 mmol), followed by palladium(II) acetate (164 mg, 0.730 mmol). The reaction mixture was heated in microwave under high absorption at 150° C. for 2 h. The reaction mixture was passed through celite and washed with EtOAc. The filtrate was washed with water (2×) and brine (1×). The organic layer was collected and concentrated to give the crude product. The crude product was purified by flash chromatography to give the title compound (662 mg, 2.70 mmol, 55.5% yield) and a less pure batch (481 mg, 1.961 mmol, 40.3% yield). LC-MS m/z 246.1 (M+H)⁺, 0.85 min (ret. time)

### Ethyl 3-(1,4-dimethyl-1H-benzo[d][1,2,3]triazol-5-yl)-3-(3-(hydroxymethyl)-4-methyl phenyl)propenoate

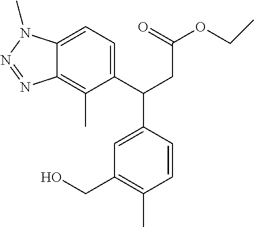 [SMILES: CCOC(=O)CC(c1ccc(C)c(CO)c1)c1ccc2c(nnn2C)c1C]

To a suspension of (E)-ethyl 3-(1,4-dimethyl-1H-benzo[d][1,2,3]triazol-5-yl)acrylate (1200 mg, 4.89 mmol), (3-(hydroxymethyl)-4-methylphenyl)boronic acid (974 mg, 5.87 mmol), and [RhCl(cod)]₂ (271 mg, 0.489 mmol) in 1,4-dioxane (10 mL) and water (10 mL) at ambient temperature was added triethylamine (2.046 mL, 14.68 mmol). The resulting suspension was heated in a Biotage microwave at high absorption for 60 minutes at 150° C. The reaction mixture was passed through celite and washed with EtOAc. The filtrate was washed with water (2×), brine (1×). The organic layer was collected and concentrated to give the crude product. The crude product was purified by flash chromatography to give the title compound (1290 mg, 3.51 mmol, 71.8% yield) and a less pure batch (453 mg). LCMS m/z 367.8 (M+H)⁺, 0.86 min (ret. time)

### Ethyl 3-(1,4-dimethyl-1H-benzo[d][1,2,3]triazol-5-yl)-3-(4-methyl-3-((methylamino)methyl)phenyl)propanoate

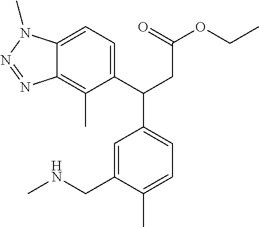 [SMILES: CCOC(=O)CC(c1ccc(C)c(CNC)c1)c1ccc2c(nnn2C)c1C]

To a solution of ethyl 3-(1,4-dimethyl-1H-benzo[d][1,2,3]triazol-5-yl)-3-(3-(hydroxymethyl)-4-methylphenyl)propanoate (1.156 g, 3.15 mmol) and triethylamine (2.180 mL, 15.73 mmol) in dichloromethane (DCM) (18 mL) at −78° C. was added gradually methanesulfonyl chloride (0.613 mL, 7.87 mmol). The mixture was then stirred for 40 mins at the same temperature under a nitrogen atmosphere. Methanamine 2M in THF (15.73 mL, 31.5 mmol) was then added and the mixture is left to return to ambient temperature while stirring for a further 1.5 h. The mixture was then taken up in DCM and washed with an aqueous solution of NaCl (4×) and NaHCO₃. The organic phase was dried over sodium sulfate, filtered and concentrated down under reduced pressure to provide the title compound which was used without purification. (1.12 g, 58 yield). LC-MS m/z 381 (M+H)⁺, 0.69 min (ret. time).

### 1-Methoxy-2-methylpropan-2-yl (4-nitrophenyl) carbonate

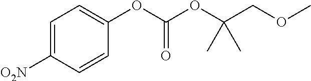 [SMILES: COCC(C)(C)OC(=O)Oc1ccc([N+](=O)[O-])cc1]

To a solution of 1-methoxy-2-methylpropan-2-ol (0.561 mL, 4.80 mmol) in pyridine (2 mL) at 0° C. was added 4-nitrophenyl carbonochloridate (1466 mg, 4.80 mmol). After adding dichloromethane (DCM) (2.5 mL), the resultant solution was stirred for 21 h at ambient temperature. After this time, the reaction was diluted with toluene and filtered. Further impurities were crystallized out from DCM-Hexane and filtered. The solvent was then removed under reduced pressure to provide the title compound (900 mg, 69% yield). ¹H NMR (400 MHz, CDCl₃)=8.31-8.26 (m, 2H), 7.41-7.36 (m, 2H), 3.59 (s, 2H), 3.45 (s, 3H), 1.59 (s, 6H).

### Ethyl 3-(1,4-dimethyl-1H-benzo[d][1,2,3]triazol-5-yl)-3-(3-(((((1-methoxy-2-methylpropan-2-yl)oxy)carbonyl)(methyl)amino)methyl)-4-methylphenyl)propanoate

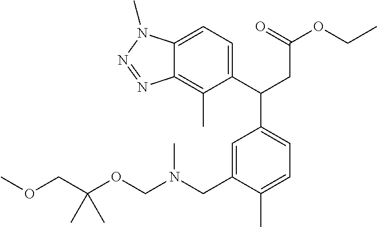 [SMILES: CCOC(=O)CC(c1ccc(C)c(CN(C)COC(C)(C)COC)c1)c1ccc2c(nnn2C)c1C]

To a solution of ethyl 3-(1,4-dimethyl-1H-benzo[d][1,2,3]triazol-5-yl)-3-(4-methyl-3-((methylamino)methyl)phenyl)propanoate (94 mg, 0.247 mmol) in pyridine (2 mL) was added 1-methoxy-2-methylpropan-2-yl(4-nitrophenyl)carbonate (86 mg, 0.321 mmol) and the mixture was left to stir at ambient temperature for 16.5 h. After this time additional 1-methoxy-2-methylpropan-2-yl(4-nitrophenyl)carbonate (33.3 mg, 0.124 mmol) was added and the mixture stirred for a further 24.5 h. The reaction was then diluted with EtOAc and washed (3×) with 10% sodium bisulfate solution. The organic phase was dried over sodium sulfate, filtered and the solvent removed under reduced pressure. The residue was purified by flash chromatography eluting 0-70% EtOAc/Hexanes. The resulting impure residue was purified by reverse phase preparative HPLC under neutral conditions to provide the title compound. (49 mg, 38% yield) LC-MS m/z 511 (M+H)⁺, 1.10 min (ret. time).

### 3-(1,4-Dimethyl-1H-benzo[d][1,2,3]triazol-5-yl)-3-(3-(((((1-methoxy-2-methylpropan-2-yl)oxy)carbonyl)(methyl)amino)methyl)-4-methylphenyl)propanoic acid

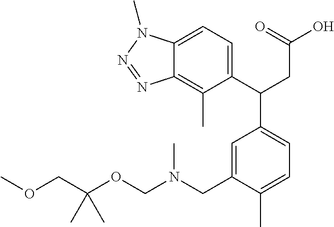 [SMILES: COCC(C)(C)OCN(C)Cc1cc(C(CC(=O)O)c2ccc3c(nnn3C)c2C)ccc1C]

To a solution of ethyl 3-(1,4-dimethyl-1H-benzo[d][1,2,3]triazol-5-yl)-3-(3-(((((1-methoxy-2-methylpropan-2-yl)oxy)carbonyl)(methyl)amino)methyl)-4-methylphenyl)propanoate (49 mg, 0.096 mmol) in tetrahydrofuran (THF) (0.7 mL) and water (0.350 mL) was added LiOH (11.49 mg, 0.480 mmol) and the mixture left to stir at ambient temperature for 17.5 h after which LiOH (11.49 mg, 0.480 mmol) and water (0.5 mL) was added and the reaction left to stir for a further 23.5 h. After this time, 1 N HCl was added to the reaction mixture dropwise until the mixture was at pH 1. The acidic solution was diluted with EtOAc, washed with water (3×), dried over magnesium sulfate, filtered and the solvents removed under reduced pressure to afford a white solid. The solid was purified by reverse phase preparative HPLC under neutral conditions to provide the title compound. (36 mg, 78% yield) LC-MS m/z 483 (M+H)⁺, 0.93 min (ret. time).

### Example 11

### 3-(3-((N-(Cyclohexylmethyl)acetamido)methyl)-4-methylphenyl)-3-(1,4-dimethyl-1H-benzo[d][1,2,3]triazol-5-yl)propanoic acid

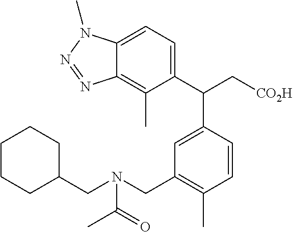 [SMILES: CC(=O)N(Cc1cc(C(CC(=O)O)c2ccc3c(nnn3C)c2C)ccc1C)CC1CCCCC1]

### Ethyl 3-(3-(chloromethyl)-4-methylphenyl)-3-(1,4-dimethyl-1H-benzo[d][1,2,3]triazol-5-yl)propanoate

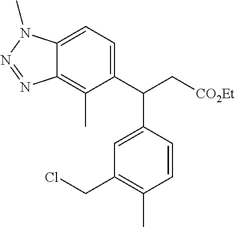 [SMILES: CCOC(=O)CC(c1ccc(C)c(CCl)c1)c1ccc2c(nnn2C)c1C]

To the solution of ethyl 3-(1,4-dimethyl-1H-benzo[d][1,2,3]triazol-5-yl)-3-(3-(hydroxymethyl)-4-methylphenyl)propanoate (440 mg, 1.197 mmol) in dichloromethane (DCM) (2 mL) was added SOCl₂ (0.175 mL, 2.395 mmol). The resulting reaction mixture was stirred at ambient temperature for 20 min. The reaction mixture was concentrated under reduced pressure to afford desired product ethyl 3-(3-(chloromethyl)-4-methylphenyl)-3-(1,4-dimethyl-1H-benzo[d][1,2,3]triazol-5-yl)propanoate (515.0 mg, 1.335 mmol, 111% yield). LC-MS m/z 386.3 (M+H)⁺, 1.13 min (ret. time).

### 3-(3-((N-(cyclohexylmethyl)acetamido)methyl)-4-methylphenyl)-3-(1,4-dimethyl-1H-benzo[d][1,2,3]triazol-5-yl)propanoic acid

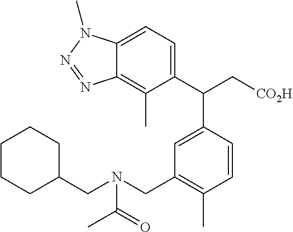 [SMILES: CC(=O)N(Cc1cc(C(CC(=O)O)c2ccc3c(nnn3C)c2C)ccc1C)CC1CCCCC1]

To the solution of ethyl 3-(3-(chloromethyl)-4-methylphenyl)-3-(1,4-dimethyl-1H-benzo[d][1,2,3]triazol-5-yl)propanoate (50 mg, 0.130 mmol) in tetrahydrofuran (THF) (1 mL) was added cyclohexylmethanamine (0.020 mL, 0.155 mmol), DIEA (0.068 mL, 0.389 mmol). The resulting reaction mixture was stirred at ambient temperature for 1 h. The reaction mixture was heated again with microwave at 80° C. for 30 min. To the reaction mixture was added acetonitrile (0.5 mL) and then heated again with microwave at 80° C. for 30 min then heated again with microwave at 100° C. for 1 h. To the reaction mixture was added AcCl (0.011 mL, 0.155 mmol) then stirred at ambient temperature for 30 min. The reaction mixture was concentrated under reduced pressure. To this crude mixture was added methanol (2 mL) and NaOH (2 N) (0.324 mL, 0.648 mmol). The resulting reaction mixture was heated with microwave at 80° C. for 20 min. The resulting reaction mixture acidified with HCl (˜0.11 mL, 6 N), concentrated under reduced pressure and purified with reverse phase HPLC to afford desired product 3-(3-((N-(cyclohexylmethyl)acetamido)methyl)-4-methylphenyl)-3-(1,4-dimethyl-1H-benzo[d][1,2,3]triazol-5-yl)propanoic acid (21.8 mg, 0.046 mmol, 35.3% yield). LC-MS m/z 476.9 (M+H)⁺, 1.03 min (ret. time).

### Example 12

### 3-(3-((N-Benzylacetamido)methyl)-4-methylphenyl)-3-(1,4-dimethyl-1H-benzo[d][1,2,3]triazol-5-yl)propanoic acid

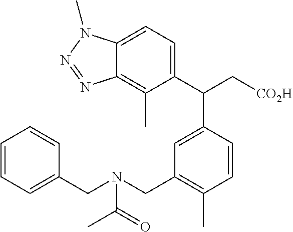 [SMILES: CC(=O)N(Cc1ccccc1)Cc1cc(C(CC(=O)O)c2ccc3c(nnn3C)c2C)ccc1C]

To the solution of ethyl 3-(3-(chloromethyl)-4-methylphenyl)-3-(1,4-dimethyl-1H-benzo[d][1,2,3]triazol-5-yl)propanoate (50 mg, 0.130 mmol) in acetonitrile (1 mL) was added benzylamine (0.028 mL, 0.259 mmol), DIEA (0.068 mL, 0.389 mmol) and Nal (1.942 mg, 0.013 mmol). The resulting reaction mixture was heated with microwave at 60° C. for 30 min, heated again with microwave at 80° C. for 30 min. To the reaction mixture was added AcCl (0.018 mL, 0.259 mmol) and then stirred at ambient temperature for 40 min. To the reaction mixture was added more DIEA (0.023 mL, 0.130 mmol) and stirred at ambient temperature for 20 min. To the reaction mixture was added more AcCl (9.21 μL, 0.130 mmol) and stirred at ambient temperature for 10 min. To the reaction mixture was added NaOH (2 N) (0.389 mL, 0.777 mmol) then heated with microwave at 80° C. for 30 min, heated again with microwave at 100° C. for 30 min. The reaction mixture was acidified with HCl (3 N) to pH ˜4-5, concentrated under reduced pressure and purified with reverse phase HPLC to afford desired product 3-(3-((N-benzylacetamido)methyl)-4-methylphenyl)-3-(1,4-dimethyl-1H-benzo[d][1,2,3]triazol-5-yl)propanoic acid (26.1 mg, 0.055 mmol, 42.8% yield). LC-MS m/z 471.3 (M+H)⁺, 0.94 min (ret. time).

### Example 13

### 3-(1,4-Dimethyl-1H-benzo[d][1,2,3]triazol-5-yl)-3-(4-methyl-3-((N-(3-methylbenzyl)acetamido)methyl)phenyl)propanoic acid

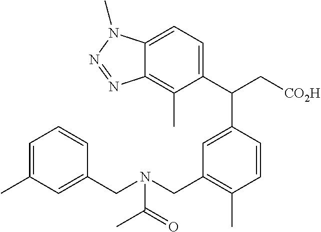 [SMILES: CC(=O)N(Cc1cccc(C)c1)Cc1cc(C(CC(=O)O)c2ccc3c(nnn3C)c2C)ccc1C]

To the solution of ethyl 3-(3-(chloromethyl)-4-methylphenyl)-3-(1,4-dimethyl-1H-benzo[d][1,2,3]triazol-5-yl)propanoate (50 mg, 0.130 mmol) in acetonitrile (4 mL) was added 3-methylbenzylamine (0.033 mL, 0.259 mmol) and DIEA (0.091 mL, 0.518 mmol). The resulting reaction mixture was heated with microwave at 80° C. for 30 min, heated again with microwave at 100° C. for 1 h. To the reaction mixture was added AcCl (0.018 mL, 0.259 mmol) and then stirred at ambient temperature for 20 min. The reaction mixture was evaporated down, re-dissolved in methanol (2 mL) then was added NaOH (2 N) (0.389 mL, 0.777 mmol). The resulting reaction mixture was heated with microwave at 80° C. for 20 min. The reaction mixture was then acidified with HCl (3 N) to pH 4˜5, concentrated under reduced pressure and purified with reverse phase HPLC to afford desired product 3-(1,4-dimethyl-1H-benzo[d][1,2,3]triazol-5-yl)-3-(4-methyl-3-((N-(3-methylbenzyl)acetamido)methyl)phenyl)propanoic acid (27.4 mg, 0.057 mmol, 43.6% yield). LC-MS m/z 485.3 (M+H)⁺, 0.98 min (ret. time).

### Example 14

### 3-(1,4-Dimethyl-1H-benzo[d][1,2,3]triazol-5-yl)-3-(3-((N-(2,3-dimethylbenzyl)acetamido)methyl)-4-methylphenyl)propanoic acid

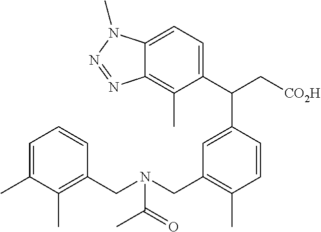 [SMILES: CC(=O)N(Cc1cc(C(CC(=O)O)c2ccc3c(nnn3C)c2C)ccc1C)Cc1cccc(C)c1C]

To the solution of ethyl 3-(3-(chloromethyl)-4-methylphenyl)-3-(1,4-dimethyl-1H-benzo[d][1,2,3]triazol-5-yl)propanoate (50 mg, 0.130 mmol) in acetonitrile (4 mL) was added 2,3-dimethylbenzylamine (35.0 mg, 0.259 mmol) and DIEA (0.091 mL, 0.518 mmol). The resulting reaction mixture was heated with microwave at 100° C. for 30 min, heated again with microwave at 100° C. for 30 min. To the reaction mixture was added AcCl (0.018 mL, 0.259 mmol) and then stirred at ambient temperature for 40 min. The reaction mixture was evaporated down over, re-dissolved in methanol (2 mL) before was added NaOH (2 N) (0.389 mL, 0.777 mmol) and then heated with microwave at 80° C. for 20 min. The reaction mixture was acidified with HCl (3 N) to pH 4˜5, concentrated under reduced pressure and purified with reverse phase HPLC to afford desired product 3-(1,4-dimethyl-1H-benzo[d][1,2,3]triazol-5-yl)-3-(3-((N-(2,3-dimethylbenzyl)acetamido)methyl)-4-methylphenyl)propanoic acid (36.6 mg, 0.073 mmol, 56.7% yield). LC-MS m/z 499.4 (M+H)⁺, 1.02 min (ret. time).

### Example 15

### 3-(1,4-Dimethyl-1H-benzo[d][1,2,3]triazol-5-yl)-3-(3-((N-(4-methoxybenzyl)acetamido)methyl)-4-methylphenyl)propanoic acid

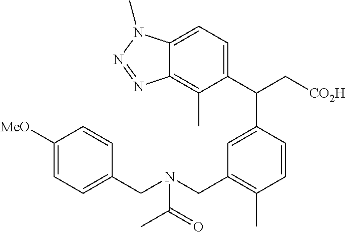 [SMILES: COc1ccc(CN(Cc2cc(C(CC(=O)O)c3ccc4c(nnn4C)c3C)ccc2C)C(C)=O)cc1]

To the solution of ethyl 3-(3-(chloromethyl)-4-methylphenyl)-3-(1,4-dimethyl-1H-benzo[d][1,2,3]triazol-5-yl)propanoate (50 mg, 0.130 mmol) in acetonitrile (4 mL) was added 4-methoxybenzylamine (0.034 mL, 0.259 mmol) and DIEA (0.091 mL, 0.518 mmol). The resulting reaction mixture was heated with microwave at 60° C. for 60 min; heated again with microwave at 70° C. for 60 min; heated again with microwave at 70° C. for 60 min. To the reaction mixture was added AcCl (0.018 mL, 0.259 mmol) and then stirred at ambient temperature for 30 min. The reaction mixture was evaporated down, re-dissolved in methanol (2 mL) before was added NaOH (2 N) (0.389 mL, 0.777 mmol) and then heated with microwave at 80° C. for 20 min. The reaction mixture was acidified with HCl (3 N) to pH ˜4-5, concentrated under reduced pressure and purified with reverse phase HPLC to afford desired product 3-(1,4-dimethyl-1H-benzo[d][1,2,3]triazol-5-yl)-3-(3-((N-(4-methoxybenzyl)acetamido)methyl)-4-methylphenyl)propanoic acid (12.6 mg, 0.025 mmol, 19.43% yield). LC-MS m/z 501.2 (M+H)⁺, 0.93 min (ret. time).

### Example 16

### 3-(1,4-Dimethyl-1H-benzo[d][1,2,3]triazol-5-yl)-3-(3-((N-(4-ethylbenzyl)acetamido)methyl)-4-methylphenyl)propanoic acid

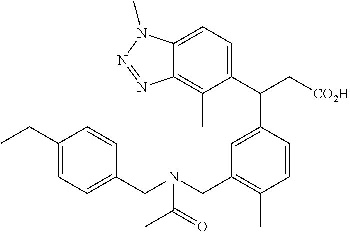 [SMILES: CCc1ccc(CN(Cc2cc(C(CC(=O)O)c3ccc4c(nnn4C)c3C)ccc2C)C(C)=O)cc1]

To the solution of ethyl 3-(3-(chloromethyl)-4-methylphenyl)-3-(1,4-dimethyl-1H-benzo[d][1,2,3]triazol-5-yl)propanoate (90 mg, 0.233 mmol) in acetonitrile (8 mL) was added 4-ethylbenzylamine (0.067 mL, 0.466 mmol) and DIEA (0.163 mL, 0.933 mmol). The resulting reaction mixture was heated with microwave at 80° C. for 60 min, heated again with microwave at 80° C. for 60 min. To the reaction mixture was added AcCl (0.033 mL, 0.466 mmol) and then stirred at ambient temperature for 100 min. The reaction mixture was evaporated down, re-dissolved in methanol (4 mL) before was added NaOH (2 N) (0.700 mL, 1.399 mmol) and then heated with microwave at 80° C. for 20 min. The reaction mixture was acidified with HCl (3 N) to pH ˜4-5, concentrated under reduced pressure and purified with reverse phase HPLC to afford desired product 3-(1,4-dimethyl-1H-benzo[d][1,2,3]triazol-5-yl)-3-(3-((N-(4-ethylbenzyl)acetamido) methyl)-4-methylphenyl)propanoic acid (59.7 mg, 0.120 mmol, 51.3% yield). LC-MS m/z 499.4 (M+H)⁺, 1.03 min (ret. time).

### Example 17

### 3-(1,4-Dimethyl-1H-benzo[d][1,2,3]triazol-5-yl)-3-(3-((N-((4-ethylcyclohexyl)methyl)acetamido)methyl)-4-methylphenyl)propanoic acid

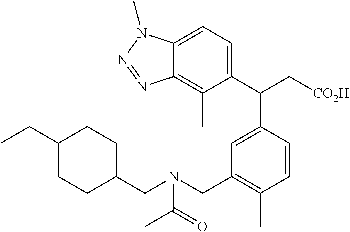 [SMILES: CCC1CCC(CN(Cc2cc(C(CC(=O)O)c3ccc4c(nnn4C)c3C)ccc2C)C(C)=O)CC1]

### 4-Ethylcyclohexanecarbonitrile

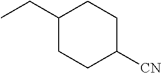

To the solution of 4-ethylcyclohexanone (1.128 mL, 8 mmol) and p-toluenesulfonylmethyl isocyanide (1874 mg, 9.60 mmol) in tetrahydrofuran (THF) (30 mL) was added KOtBu (1795 mg, 16.00 mmol) slowly at 0° C. The resulting reaction mixture was stirred at 0° C. for 25 min then ambient temperature for 2 h. The reaction mixture was concentrated under reduced pressure. To this crude mixture was added water (100 mL), extracted with hexane (3×50 mL). The combined organic layer was washed with brine (50 mL), dried over MgSO₄, filtered, concentrated under reduced pressure and purified with flash chromatograph over silica gel column to afford desired product 4-ethylcyclohexanecarbonitrile (611.3 mg, 4.45 mmol, 55.7% yield). LC-MS m/z 138.3 (M+H)⁺, 0.95 min (ret. time).

### (4-Ethylcyclohexyl)methanamine

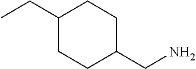

To the suspension of LAH (131 mg, 3.45 mmol) in tetrahydrofuran (THF) (5 mL) was added 4-ethylcyclohexanecarbonitrile (316 mg, 2.3 mmol) in tetrahydrofuran (THF) (1 mL) slowly at ambient temperature. The resulting reaction mixture was stirred at ambient temperature for 50 min. The reaction mixture was quenched slowly with Na₂SO₄ (sat., aq.), filtered, concentrated under reduced pressure to afford desired product (4-ethylcyclohexyl)methanamine (292.7 mg, 2.072 mmol, 90% yield). LC-MS m/z 142.1 (M+H)⁺, 0.67 min (ret. time).

### 3-(1,4-Dimethyl-1H-benzo[d][1,2,3]triazol-5-yl)-3-(3-((N-((4-ethylcyclohexyl)methyl)acetamido)methyl)-4-methylphenyl)propanoic acid

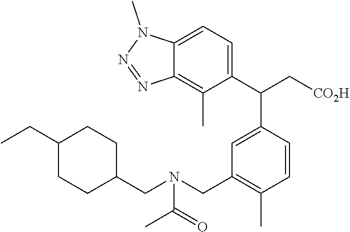 [SMILES: CCC1CCC(CN(Cc2cc(C(CC(=O)O)c3ccc4c(nnn4C)c3C)ccc2C)C(C)=O)CC1]

To the solution of ethyl 3-(3-(chloromethyl)-4-methylphenyl)-3-(1,4-dimethyl-1H-benzo[d][1,2,3]triazol-5-yl)propanoate (50 mg, 0.130 mmol) in acetonitrile (2 mL) was added (4-ethylcyclohexyl)methanamine (36.6 mg, 0.259 mmol), DIEA (0.091 mL, 0.518 mmol). The resulting reaction mixture was heated with microwave at 80° C. for 60 min. To the reaction mixture was added AcCl (0.018 mL, 0.259 mmol) and stirred at ambient temperature for 15 min. To the reaction mixture was added NaOH (3.0 N) (0.346 mL, 1.037 mmol) then heated with microwave at 80° C. for 30 min, heated again with microwave at 80° C. for 30 min, heated again with microwave at 80° C. for 30 min, heated again with microwave at 80° C. for 30 min. The reaction mixture was then concentrated under reduced pressure and purified with reverse phase HPLC to afford desired product 3-(1,4-dimethyl-1H-benzo[d][1,2,3]triazol-5-yl)-3-(3-((N-((4-ethylcyclohexyl)methyl)acetamido)methyl)-4-methylphenyl)propanoic acid (7.1 mg, 0.014 mmol, 10.86% yield). LC-MS m/z 505.3 (M+H)⁺, 1.12 min (ret. time).

### Example 18

### 3-(3-((1-(Cyclohexylmethyl)ureido)methyl)-4-methylphenyl)-3-(1,4-dimethyl-1H-benzo[d][1,2,3]triazol-5-yl)propanoic acid

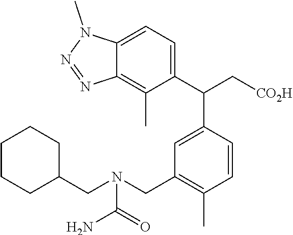 [SMILES: Cc1ccc(C(CC(=O)O)c2ccc3c(nnn3C)c2C)cc1CN(CC1CCCCC1)C(N)=O]

To the solution of cyclohexylmethanamine (0.034 mL, 0.259 mmol) in acetonitrile (2 mL) was added DIEA (0.091 mL, 0.518 mmol) and then ethyl 3-(3-(chloromethyl)-4-methylphenyl)-3-(1,4-dimethyl-1H-benzo[d][1,2,3]triazol-5-yl)propanoate (50 mg, 0.130 mmol). The resulting reaction mixture was heated with microwave at 80° C. for 60 min. To the reaction mixture was then added TMS-NCO (0.035 mL, 0.259 mmol) and stirred at ambient temperature for 40 min. The reaction mixture was then heated with microwave at 80° C. for 30 min. To the reaction mixture was added more TMS-NCO (0.018 mL, 0.130 mmol) and then heated with microwave at 80° C. for 15 min. The reaction mixture was evaporated down on under vacuum, re-dissolved in methanol (2.000 mL) then was added NaOH (3.0 N) (0.346 mL, 1.037 mmol). The resulting reaction mixture was heated with microwave at 80° C. for 20 min. The reaction mixture was acidified with HCl (3 N) to pH 4˜5, evaporated down on under vacuum, purified with reverse phase HPLC to afford desired product 3-(3-((1-(cyclohexylmethyl)ureido)methyl)-4-methylphenyl)-3-(1,4-dimethyl-1H-benzo[d][1,2,3]triazol-5-yl)propanoic acid (27.1 mg, 0.057 mmol, 43.8% yield). LC-MS m/z 478.2 (M+H)⁺, 0.97 min (ret. time).

### Example 19

### 3-(1,4-Dimethyl-1H-benzo[d][1,2,3]triazol-5-yl)-3-(3-((1-((4-ethylcyclohexyl)methyl)ureido)methyl)-4-methylphenyl)propanoic acid

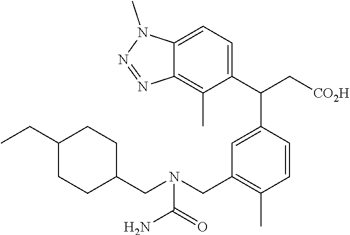 [SMILES: CCC1CCC(CN(Cc2cc(C(CC(=O)O)c3ccc4c(nnn4C)c3C)ccc2C)C(N)=O)CC1]

To the solution of (4-ethylcyclohexyl)methanamine (36.6 mg, 0.259 mmol) in acetonitrile (2 mL) was added DIEA (0.091 mL, 0.518 mmol) and then ethyl 3-(3-(chloromethyl)-4-methylphenyl)-3-(1,4-dimethyl-1H-benzo[d][1,2,3]triazol-5-yl)propanoate (50 mg, 0.130 mmol). The resulting reaction mixture was heated with microwave at 80° C. for 60 min before was added TMS-NCO (0.053 mL, 0.389 mmol) and stirred at 80° C. for 20 min. The reaction mixture was evaporated down, re-dissolved in methanol (2.000 mL) then was added NaOH (3.0 N) (0.346 mL, 1.037 mmol). The resulting reaction mixture was heated with microwave at 80° C. for 20 min. The reaction mixture was acidified with HCl (3N) to pH ˜4-5, concentrated under reduced pressure and purified with reverse phase HPLC to afford desired product 3-(1,4-dimethyl-1H-benzo[d][1,2,3]triazol-5-yl)-3-(3-((1-((4-ethylcyclohexyl)methyl)ureido)methyl)-4-methylphenyl)propanoic acid (16.6 mg, 0.033 mmol, 25.3% yield). LC-MS m/z 506.3 (M+H)⁺, 1.09 min (ret. time).

### Example 20

### 3-(3-((N-(Cyclohexylmethyl)acetamido)methyl)-4-methylphenyl)-3-(7-methoxy-1-methyl-1H-benzo[d][1,2,3]triazol-5-yl)propanoic acid

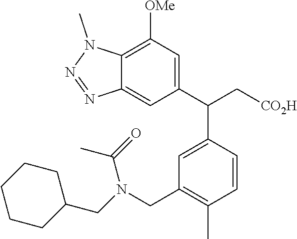 [SMILES: COc1cc(C(CC(=O)O)c2ccc(C)c(CN(CC3CCCCC3)C(C)=O)c2)cc2nnn(C)c12]

### Ethyl 3-(3-(hydroxymethyl)-4-methylphenyl)-3-(7-methoxy-1-methyl-1H-benzo[d][1,2,3]triazol-5-yl)propanoate

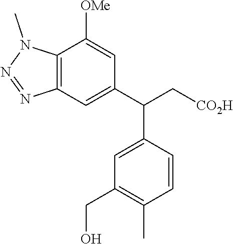 [SMILES: COc1cc(C(CC(=O)O)c2ccc(C)c(CO)c2)cc2nnn(C)c12]

To a solution of (E)-ethyl 3-(7-methoxy-1-methyl-1H-benzo[d][1,2,3]triazol-5-yl)acrylate (2 g, 7.65 mmol) and (3-(hydroxymethyl)-4-methylphenyl)boronic acid (3.177 g, 19.13 mmol) dissolved in 1,4-dioxane (6 mL) and water (6.00 mL) were added [RhCl(cod)]₂ (0.863 g, 1.56 mmol) and TEA (4.4 mL, 31.8 mmol) and the mixture was heated in microwave at 125° C. for 10 h. The mixture was filtered, washed with ethyl acetate; then the filtrate was washed with water (2×) and brine once. The organic layer was dried over MgSO₄, concentrated and purified through silica gel chromatography to get 885 mg of ethyl 3-(3-(hydroxymethyl)-4-methylphenyl)-3-(7-methoxy-1-methyl-1H-benzo[d][1,2,3]triazol-5-yl)propanoate (30.2%). LC-MS m/z 384.1 (M+H)⁺, 0.92 (ret. time).

### Ethyl 3-(3-(chloromethyl)-4-methylphenyl)-3-(7-methoxy-1-methyl-1H-benzo[d][1,2,3]triazol-5-yl)propanoate

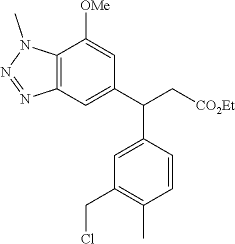 [SMILES: CCOC(=O)CC(c1ccc(C)c(CCl)c1)c1cc(OC)c2c(c1)nnn2C]

To a stirred solution of ethyl 3-(3-(hydroxymethyl)-4-methylphenyl)-3-(7-methoxy-1-methyl-1H-benzo[d][1,2,3]triazol-5-yl)propanoate (200 mg, 0.522 mmol) dissolved in (DCM) (2 mL) was added sulfurous dichloride (0.057 mL, 0.782 mmol) and the mixture was stirred at ambient temperature for 45 min. The solvent was removed to afford 219 mg of ethyl 3-(3-(chloromethyl)-4-methylphenyl)-3-(7-methoxy-1-methyl-1H-benzo[d][1,2,3]triazol-5-yl)propanoate (104% yield). LC-MS m/z 402.2 (M+H)⁺, 1.11 (ret. time).

### 3-(3-((N-(Cyclohexylmethyl)acetamido)methyl)-4-methylphenyl)-3-(7-methoxy-1-methyl-1H-benzo[d][1,2,3]triazol-5-yl)propanoic acid

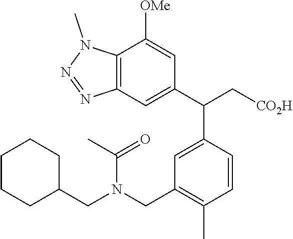 [SMILES: COc1cc(C(CC(=O)O)c2ccc(C)c(CN(CC3CCCCC3)C(C)=O)c2)cc2nnn(C)c12]

To ethyl 3-(3-(chloromethyl)-4-methylphenyl)-3-(7-methoxy-1-methyl-1H-benzo[d][1,2,3]triazol-5-yl)propanoate (40 mg, 0.100 mmol) dissolved in THF (3 mL) was added cyclohexylmethanamine (0.065 mL, 0.498 mmol) and DIEA (0.035 mL, 0.199 mmol) and the mixture was stirred at ambient temperature for 151 h. Acetyl chloride (0.014 mL, 0.199 mmol) and DIEA (0.087 mL, 0.498 mmol) were added and the mixture was stirred at ambient temperature for 4 h then sodium hydroxide 1N (0.750 mL, 0.750 mmol) was added and the mixture was heated in microwave at 100° C. for 45 min. The mixture was acidified with HCl 1N until pH=2-3, concentrated and purified on reverse phase HPLC (Sunfire C18, 19×100 mm, 5 u column), eluting at 18 mL/min with a linear gradient running from 40% CH₃CN/H₂O (0.1% formic acid) to 60% CH₃CN/H₂O (0.1% formic acid) over 10 min. The desired fractions were collected and dried by V10 solvent evaporator. Dried fractions were concentrated to give 21.8 mg of 3-(3-((N-(cyclohexylmethyl)acetamido)methyl)-4-methylphenyl)-3-(7-methoxy-1-methyl-1H-benzo[d][1,2,3]triazol-5-yl)propanoic acid (44.5% yield). LC-MS m/z 493.3 (M+H)⁺, 1.02 (ret. time).

### Example 21

### 3-(3-((2-(1-Carboxyethoxy)benzamido)methyl)-4-methylphenyl)-3-(1,4-dimethyl-1H-benzo[d][1,2,3]triazol-5-yl)propanoic acid

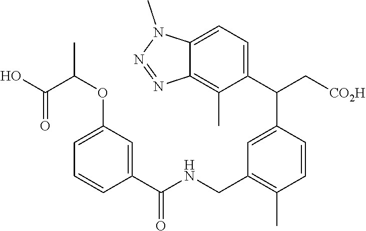 [SMILES: Cc1ccc(C(CC(=O)O)c2ccc3c(nnn3C)c2C)cc1CNC(=O)c1cccc(OC(C)C(=O)O)c1]

To a solution of ethyl 3-(1,4-dimethyl-1H-benzo[d][1,2,3]triazol-5-yl)-3-(3-(hydroxymethyl)-4-methylphenyl)propanoate (80 mg, 0.218 mmol), 2-methylbenzo[f][1,4]oxazepine-3,5(2H,4H)-dione (62.4 mg, 0.327 mmol), and 1,1′-(azodicarbonyl)dipiperidine (73.0 mg, 0.289 mmol) in tetrahydrofuran (THF) (3 mL) at ambient temperature was added tri-n-butylphosphine (0.107 mL, 0.435 mmol). The reaction mixture was stirring at ambient temperature for 20 h. Solvent was concentrated and purified by reverse-phase HPLC to give the intermediate. The intermediate was redissolved in methanol (2 mL), 2M LiOH (0.653 mL, 1.306 mmol) was added and heated in Biotage microwave at high absorption for 30 minutes at 80° C. 0.8 mL of 1 N HCl and 1.5 mL of DMSO were added. Most solvents were concentrated and then purified by reverse-phase HPLC to give the title compound (53 mg, 0.100 mmol, 45.9% yield). LC-MS m/z 531.0 (M+H)⁺, 0.82 min (ret. time)

### Intermediate 11

### 1-Oxaspiro[2.6]nonane

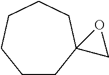

To a suspension of cycloheptanone (10 g, 89 mmol) and trimethyl sulfoxonium iodide (39.2 g, 178 mmol) in DMSO (100 mL) was added potassium tert-butoxide (20.01 g, 178 mmol). The reaction mixture was stirred under argon at 20° C. for 16 h. The mixture was extracted with ethyl acetate (2×200 mL)/H₂O (100 mL). The organic phase was washed with water (200 mL), dried over Na₂SO₄ and concentrated to give the title product (10 g, 71.3 mmol, 80% yield). ¹H-NMR (CDCl₃, 500 MHz); 2.56 (s, 2H), 1.69-1.63 (m, 6H), 1.61-1.50 (m, 6H).

### 1-(Aminomethyl)cycloheptanol

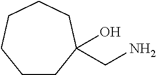

To the solution of 1-oxaspiro[2.6]nonane (10 g, 79 mmol) in methanol (10 mL)/water (10 mL) was added ammonium hydroxide (30.9 mL, 792 mmol). The reaction mixture was stirred at 50° C. for 16 h. The organic solvent was removed. The residue was extracted with ethyl acetate (2×100 mL). The organic phase was washed with water (100 mL), dried over Na₂SO₄ and then concentrated. The residue was purified by flash chromatograph to give the title compound (2.3 g, 14.45 mmol, 18.24% yield). LC-MS m/z 0.98 (M+H)⁺, 0.98 min (ret. time).

The compounds in Table 1 were prepared by a method similar to the one described for the preparation of 1-(Aminomethyl)cycloheptanol. As is appreciated by those skilled in the art, these analogous examples may involve variations in general reaction conditions.

TABLE 1

| | | | Retention |
|---|---|---|---|
| | | LCMS | Time |
| Structure | Name | [M + H]⁺ | (min) |
| 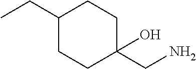 | 1-(Amino- methyl)-4- ethylcyclo- hexanol | 158.2 | 1.13 |

### Example 22

### 3-(1,4-Dimethyl-1H-benzo[d][1,2,3]triazol-5-yl)-3-(3-((1-((4-ethylcyclohexyl)methyl)ureido)methyl)-4-methylphenyl)-2,2-dimethylpropanoic acid

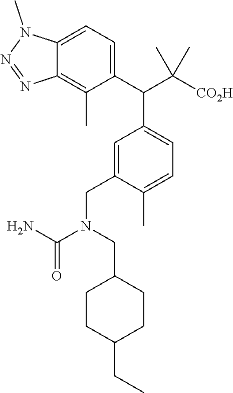 [SMILES: CCC1CCC(CN(Cc2cc(C(c3ccc4c(nnn4C)c3C)C(C)(C)C(=O)O)ccc2C)C(N)=O)CC1]

### (5-Bromo-2-methylphenyl)methanol

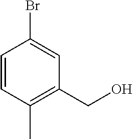

To a solution of 5-bromo-2-methylbenzoic acid (70 g, 326 mmol) in tetrahydrofuran (THF) (700 mL) stirred under nitrogen at 0° C. was added a toluene solution of borane-methyl sulfide complex (244 mL, 488 mmol) drop wise during 15 min. The reaction mixture was stirred for 16 h. The reaction was cooled to 0° C. and quenched with methanol (500 mL) drop wise. The reaction mixture was stirred at ambient temperature for 3 h and then concentrated. The crude residue was diluted with ethyl acetate (1 L) and washed with 1N HCl (500 mL), brine solution (500 mL) and dried over Na₂SO₄, filtered and concentrated to give the title compound (49 g, 244 mmol, 74.9% yield). ¹H NMR (400 MHz, DMSO) δ=7.52 (d, J=2.6 Hz, 1H), 7.31 (dd, J=8.0, 2.2 Hz, 1H), 7.12-7.03 (m, 1H), 5.22 (td, J=5.5, 1.8 Hz, 1H), 4.48 (dd, J=5.1, 1.8 Hz, 2H), 2.17 (s, 3H).

### 4-Bromo-2-(((4-methoxybenzyl)oxy)methyl)-1-methylbenzene

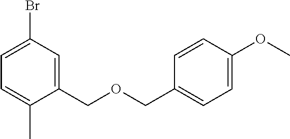 [SMILES: COc1ccc(COCc2cc(Br)ccc2C)cc1]

To a stirred solution of (5-bromo-2-methylphenyl)methanol (100 g, 497 mmol) in dry DMF (800 mL) was added NaH (21.88 g, 547 mmol). After the reaction mixture was stirred for 30 minutes, 1-(chloromethyl)-4-methoxybenzene (82 g, 522 mmol) was added at 0° C. and the reaction mixture was stirred for another 2 h at ambient temperature. The reaction was then diluted with Et₂O (200 mL) and water (200 mL). The organic phase was washed with brine (300 mL) and dried with Na₂SO₄ and concentrated under reduced pressure. The residue was purified via silica gel column to yield 4-bromo-2-(((4-methoxybenzyl)oxy)methyl)-1-methylbenzene (140 g, 436 mmol, 88% yield) as a clear oil. ¹H NMR (400 MHz, CHLOROFORM-d) δ ppm 2.27 (s, 3H) 3.84 (s, 3H) 4.49 (s, 2H), 4.54 (s, 2H), 6.92 (d, J=8.8, 2H), 6.94 (d, J=8.4, 1H), 7.31-7.35 (m, 3H), 7.54 (d, J=2, 1H).

### 3-(4-Methoxybenzyl)oxy)methyl)-4-methylbenzaldehyde

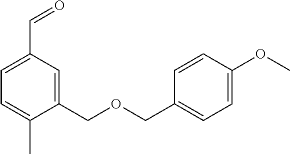 [SMILES: COc1ccc(COCc2cc(C=O)ccc2C)cc1]

To a stirred solution of 4-bromo-2-(((4-methoxybenzyl)oxy)methyl)-1-methylbenzene (80 g, 249 mmol) in THF (800 mL) at −78° C. under N₂, 2.5 M n-BuLi in hexane (120 mL, 299 mmol) was carefully added. The reaction mixture was stirred at −78° C. for 65 min, and then DMF (38.6 mL, 498 mmol) was added. The reaction mixture was stirred at −78° C. to 25° C. for another 30 min. The mixture was quenched with saturated NH₄Cl (300 mL), and extracted with EtOAc (2×500 mL). The organic layer was washed with water (300 mL) and brine (2×100 mL), dried (Na₂SO₄) and concentrated. The residue was washed with petroleum ether:EtOAc=10/1 (2000 mL) to give the title compound (50 g, 185 mmol, 74.3% yield) as a solid. LC-MS m/z 288.1 (M+H₂O)⁺, 2.04 min (ret. time).

### (1,4-Dimethyl-1H-benzo[d][1,2,3]triazol-5-yl)(3-(((4-methoxybenzyl)oxy)methyl)-4-methylphenyl)methanol

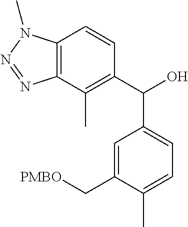

tert-Butyllithium (19.52 mL, 33.2 mmol) was added dropwise to a solution of 5-bromo-1,4-dimethyl-1H-benzo[d][1,2,3]triazole (3.91 g, 17.31 mmol) in tetrahydrofuran (THF) (108 ml) at −78° C. under a nitrogen atmosphere and stirred for 30 minutes. A solution of 3-(((4-methoxybenzyl)oxy)methyl)-4-methylbenzaldehyde (3.9 g, 14.43 mmol) in tetrahydrofuran (THF) (36.1 ml) was then added dropwise and stirred at −78° C. for 1.5 hours followed by warming to room temperature and stirring for an additional hour. Saturated aqueous ammonium chloride (100 mL) was added to the solution. Same scale reaction was runned side by side. The 2 batches were combined and the mixture was extracted with ethyl acetate (3×100 mL). The combined organic fractions were washed (brine), dried (sodium sulfate), filtered and the solvent was concentrated. The crude product was purified by silica gel chromotagraphy to give title compound (8.8 g, 21.08 mmol, 73.0% yield). LC/MS: m/z 418.0 (M+H)⁺, 1.11 min (ret. time). 1H NMR (400 MHz, CHLOROFORM-d) δ=7.76-7.69 (m, 1H), 7.34 (s, 2H), 7.24 (d, J=8.3 Hz, 2H), 7.21-7.12 (m, 2H), 6.87 (d, J=8.3 Hz, 2H), 6.27 (s, 1H), 4.49 (d, J=5.0 Hz, 4H), 4.29 (s, 3H), 3.83 (s, 3H), 2.81 (s, 3H), 2.31 (s, 3H), 2.07 (s, 1 H).

### Methyl 3-(1,4-dimethyl-1H-benzo[d][1,2,3]triazol-5-yl)-3-(3-(hydroxymethyl)-4-methylphenyl)-2,2-dimethylpropanoate

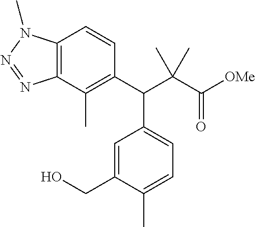 [SMILES: COC(=O)C(C)(C)C(c1ccc(C)c(CO)c1)c1ccc2c(nnn2C)c1C]

2,2,2-trichloroacetonitrile (4.23 ml, 42.2 mmol) and DBU (0.146 ml, 1.054 mmol) were added sequentially to a solution of (1,4-dimethyl-1H-benzo[d][1,2,3]triazol-5-yl)(3-(((4-methoxybenzyl)oxy)methyl)-4-methylphenyl)methanol (8.8 g, 21.08 mmol) in acetonitrile (263 ml) at ambient temperature and stirred for 45 minutes. ((1-Methoxy-2-methylprop-1-en-1-yl)oxy)trimethylsilane (9.19 g, 52.7 mmol) followed by 1,1,1-trifluoro-N-((trifluoromethyl)sulfonyl)methanesulfonamide (0.593 g, 2.108 mmol) were then added and the solution stirred at ambient temperature for 2 h. The reaction was quenched with saturated sodium bicarbonate(10 mL) and extracted with DCM (3×15 mL), dried over sodium sulfate, filtered and concentrated. The residue was redissolved in dichloromethane (DCM) (263 mL). Water (15.19 mL, 843 mmol) was added and the solution was cooled to 0° C., 4,5-dichloro-3,6-dioxocyclohexa-1,4-diene-1,2-dicarbonitrile (9.57 g, 42.2 mmol) was added. The solution was stirred at 0° C. for 1 h. The reaction was quenched with saturated sodium bicarbonate (10 mL) and extracted with DCM (3×15 mL) and dried over sodium sulfate. The crude product was purified by flash chromatography on a silica gel chromatography to give the title compound (6.9 g, 18.09 mmol, 86% yield). LC/MS: m/z 382.0 (M+H)⁺, 1.00 min (ret. time).

### Methyl 3-(3-(chloromethyl)-4-methylphenyl)-3-(1,4-dimethyl-1H-benzo[d][1,2,3]triazol-5-yl)-2,2-dimethylpropanoate

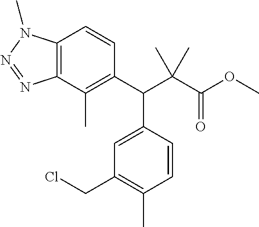 [SMILES: COC(=O)C(C)(C)C(c1ccc(C)c(CCl)c1)c1ccc2c(nnn2C)c1C]

To a solution of methyl 3-(1,4-dimethyl-1H-benzo[d][1,2,3]triazol-5-yl)-3-(3-(hydroxymethyl)-4-methylphenyl)-2,2-dimethylpropanoate (2200 mg, 5.77 mmol) in dichloromethane (DCM) (10 mL) at 25° C. was added thionyl chloride (0.842 mL, 11.53 mmol). The mixture was stirred at 25 ° C. for 40 min. The reaction mixture was concentrated to give the title compound (2200 mg, 5.50 mmol, 95% yield). LC-MS m/z 399.9 (M)⁺, 1.14 min (ret. time).

### Methyl 3-(1,4-dimethyl-1H-benzo[d][1,2,3]triazol-5-yl)-3-(3-((1-((4-ethylcyclohexyl)methyl)ureido)methyl)-4-methylphenyl)-2,2-dimethylpropanoate

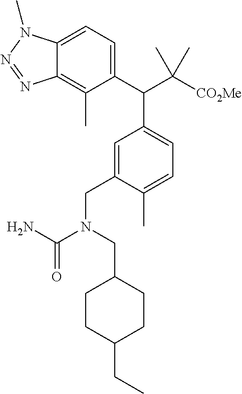 [SMILES: CCC1CCC(CN(Cc2cc(C(c3ccc4c(nnn4C)c3C)C(C)(C)C(=O)OC)ccc2C)C(N)=O)CC1]

To the mixture of methyl 3-(3-(chloromethyl)-4-methylphenyl)-3-(1,4-dimethyl-1H-benzo[d][1,2,3]triazol-5-yl)-2,2-dimethylpropanoate (60 mg, 0.150 mmol) in acetonitrile (3.0 mL) and tetrahydrofuran (THF) (1.0 mL) was added (4-ethylcyclohexyl)methanamine (85 mg, 0.600 mmol) and DIEA (0.105 mL, 0.600 mmol). The resulting reaction mixture was heated via microwave at 100° C. for 1 h before adding TMS-NCO (0.122 mL, 0.900 mmol). The resulting reaction mixture was heated via microwave at 80° C. for 20 min then quenched with H₂O (0.3 mL), concentrated under reduced pressure and purified with flash chromatograph to give the title compound (48.6 mg, 0.089 mmol, 59.1% yield). LC-MS m/z 548.5 (M+H)⁺, 1.32 (ret. time).

### 3-(1,4-Dimethyl-1H-benzo[d][1,2,3]triazol-5-yl)-3-(34((1-((4-ethylcyclohexyl)methyl)ureido)methyl)-4-methylphenyl)-2,2-dimethylpropanoic acid

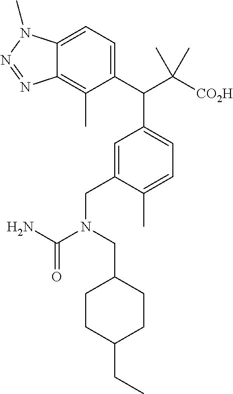 [SMILES: CCC1CCC(CN(Cc2cc(C(c3ccc4c(nnn4C)c3C)C(C)(C)C(=O)O)ccc2C)C(N)=O)CC1]

To the mixture of methyl 3-(1,4-dimethyl-1H-benzo[d][1,2,3]triazol-5-yl)-3-(3-((1-((4-ethylcyclohexyl)methyl)ureido)methyl)-4-methylphenyl)-2,2-dimethylpropanoate (48 mg, 0.088 mmol) in methanol (1.5 mL) was added NaOH (3.0 N) (0.146 mL, 0.438 mmol). The resulting reaction mixture was heated via microwave at 130° C. for 1 h 3 times. The reaction mixture was quenched with HCl (3.0 N) (0.146 mL, 0.438 mmol), concentrated under reduced pressure, and redissolved in N,N-Dimethylformamide (DMF) (1.0 mL) before adding TMS-NCO (0.071 mL, 0.526 mmol) and DIEA (0.046 mL, 0.263 mmol). The resulting reaction mixture was stirred at ambient temperature for 1 h before adding methanol (1.0 mL) and NaOH (3.0 N) (0.146 mL, 0.438 mmol). The reaction was heated via microwave at 80° C. for 20 min. Additional NaOH (3.0 N) (0.146 mL, 0.438 mmol) was added and the reaction heated via microwave at 80° C. for 20 min, then heated again with microwave at 100° C. for 20 min. The reaction mixture was quenched with HCl (3.0 N) (0.292 mL, 0.876 mmol), concentrated under reduced pressure, purified by reverse phase HPLC (TFA modifier) to give the title compound (10.3 mg, 0.019 mmol, 22.02% yield). LC-MS m/z 534.5 (M+H)⁺, 1.18 (ret. time).

### Example 23

### (S)-3-(3-((1-(Cycloheptylmethyl)ureido)methyl)-4-methylphenyl)-3-(1,4-dimethyl-1H-benzo[d][1,2,3]triazol-5-yl)-2,2-dimethylpropanoic acid

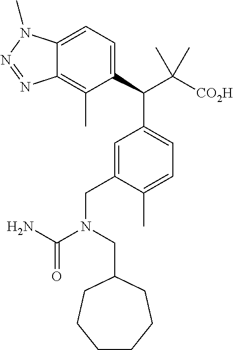 [SMILES: Cc1ccc([C@@H](c2ccc3c(nnn3C)c2C)C(C)(C)C(=O)O)cc1CN(CC1CCCCCC1)C(N)=O]

### (S)-Methyl 3-(1,4-dimethyl-1H-benzo[d][1,2,3]triazol-5-yl)-3-(3-(hydroxymethyl)-4-methylphenyl)-2,2-dimethylpropanoate and (R)-methyl 3-(1,4-dimethyl-1H-benzo[d][1,2,3]triazol-5-yl)-3-(3-(hydroxymethyl)-4-methylphenyl)-2,2-dimethylpropanoate

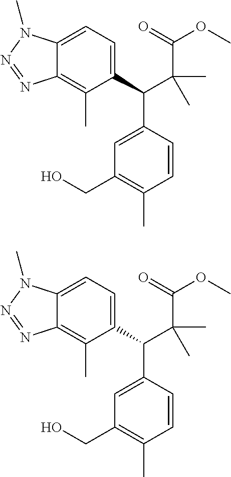 [SMILES: COC(=O)C(C)(C)[C@@H](c1ccc(C)c(CO)c1)c1ccc2c(nnn2C)c1C ; COC(=O)C(C)(C)[C@H](c1ccc(C)c(CO)c1)c1ccc2c(nnn2C)c1C]

Methyl 3-(1,4-dimethyl-1H-benzo[d][1,2,3]triazol-5-yl)-3-(3-(hydroxymethyl)-4-methylphenyl)-2,2-dimethylpropanoate (4.5 g, 11.80 mmol) was separated by chiral SFC chromatography to obtain isomer 1-(S)-methyl 3-(1,4-dimethyl-1H-benzo[d][1,2,3]triazol-5-yl)-3-(3-(hydroxymethyl)-4-methylphenyl)-2,2-dimethylpropanoate (1.4 g, 3.49 mmol, 29.6%) and isomer 2-(R)-methyl 3-(1,4-dimethyl-1H-benzo[d][1,2,3]triazol-5-yl)-3-(3-(hydroxymethyl)-4-methylphenyl)-2,2-dimethylpropanoate (1.1 g, 2.74 mmol, 23.22%).

### (S)-Methyl 3-(3-(chloromethyl)-4-methylphenyl)-3-(1,4-dimethyl-1H-benzo[d][1,2,3]triazol-5-yl)-2,2-dimethylpropanoate

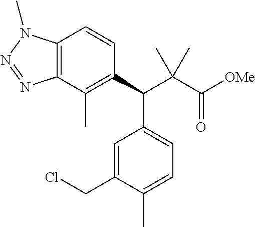 [SMILES: COC(=O)C(C)(C)[C@@H](c1ccc(C)c(CCl)c1)c1ccc2c(nnn2C)c1C]

To a solution of (S)-methyl 3-(1,4-dimethyl-1H-benzo[d][1,2,3]triazol-5-yl)-3-(3-(hydroxymethyl)-4-methylphenyl)-2,2-dimethylpropanoate (710 mg, 1.861 mmol) in dichloromethane (10 mL) at ambient temperature was added thionyl chloride (0.272 mL, 3.72 mmol). The reaction was stirred for 40 min. The resulting mixture was concentrated to give the title compound (750 mg, 1.875 mmol, 101% yield). LC-MS m/z 400.2 (M+H)⁺, 1.21 min (ret. time).

### (S)-3-(3-((1-(Cycloheptylmethyl)ureido)methyl)-4-methylphenyl)-3-(1,4-dimethyl-1H-benzo[d][1,2,3]triazol-5-yl)-2,2-dimethylpropanoic acid

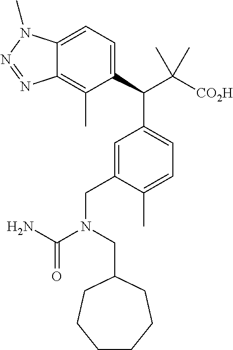 [SMILES: Cc1ccc([C@@H](c2ccc3c(nnn3C)c2C)C(C)(C)C(=O)O)cc1CN(CC1CCCCCC1)C(N)=O]

To the mixture of (S)-methyl 3-(3-(chloromethyl)-4-methylphenyl)-3-(1,4-dimethyl-1H-benzo[d][1,2,3]triazol-5-yl)-2,2-dimethylpropanoate (60 mg, 0.150 mmol) in acetonitrile (3.0 mL) and tetrahydrofuran (1.0 mL) was added cycloheptylmethanamine (0.043 mL, 0.300 mmol), and DIEA (0.052 mL, 0.300 mmol). The resulting reaction mixture was heated via microwave at 100° C. for 1 h before being concentrated under reduced pressure and the residue redissolved in methanol (2.0 mL). NaOH (3.0 N) (0.400 mL, 1.200 mmol) was added and the resulting mixture was heated via microwave at 100° C. for 1 h twice before acidifying with HCl (3.0 N) (0.400 mL, 1.200 mmol). The reaction was concentrated under reduced pressure and extracted with DCM (2×3 mL). The combined organic portion was dried over Na₂SO₄, filtered, concentrated under reduced pressure, redissolved in dichloromethane (2.0 mL) after which DIEA (0.105 mL, 0.600 mmol) and TMS-NCO (0.041 mL, 0.300 mmol) were added. The resulting reaction mixture was stirred at ambient temperature for 30 min when more TMS-NCO (10.16 μl, 0.075 mmol) was added. The resulting reaction mixture was stirred at ambient temperature for 15 min before addition of H₂O (0.3 mL) then concentrated under reduced pressure and purified by reverse phase HPLC (formic acid modifier) to give the title compound (41.8 mg, 0.080 mmol, 53.6% yield). LC-MS m/z 520.5 (M+H)⁺, 1.12 (ret. time).

The compounds in Table 2 were prepared by a method similar to the one described for the preparation of (S)-3-(3-((1-(Cycloheptylmethyl)ureido)methyl)-4-methylphenyl)-3-(1,4-dimethyl-1H-benzo[d][1,2,3]triazol-5-yl)-2,2-dimethylpropanoic acid. As is appreciated by those skilled in the art, these analogous examples may involve variations in general reaction conditions.

TABLE 2

| | | | LCMS | Retention |
|---|---|---|---|---|
| Ex # | Structure | Name | [M + H]⁺ | Time (min) |
| 24 | 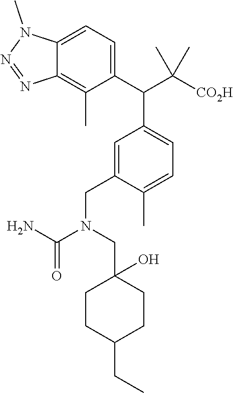 [SMILES: CCC1CCC(O)(CN(Cc2cc(C(c3ccc4c(nnn4C)c3C)C(C)(C)C(=O)O)ccc2C)C(N)=O)CC1] | (S)-3-(1,4-Dimethyl-1H- benzo[d][1,2,3]triazol-5- yl)-3-(3-((1-((4-ethyl-1- hydroxycyclo- hexyl)methyl)ureido) methyl)-4- methylphenyl)-2,2- dimethylpropanoic acid | 550.2 | 1.06 |
| 25 | 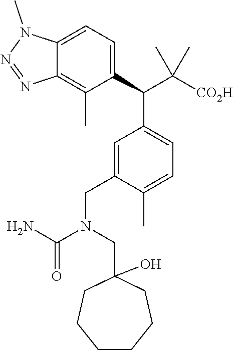 [SMILES: Cc1ccc([C@@H](c2ccc3c(nnn3C)c2C)C(C)(C)C(=O)O)cc1CN(CC1(O)CCCCCC1)C(N)=O] | (S)-3-(1,4-Dimethyl-1H- benzo[d][1,2,3]triazol-5- yl)-3-(3-((1-((1- hydroxycyclo- heptyl)methyl)ureido) methyl)-4- methylphenyl)-2,2- dimethylpropanoic acid | 536.2 | 0.99 |

### Example 26

### 3-(1,4-Dimethyl-1H-benzo[d][1,2,3]triazol-5-yl)-3-(3-((N-((1-hydroxycyclohexyl)methyl)acetamido)methyl)-4-methylphenyl)-2,2-dimethylpropanoic acid

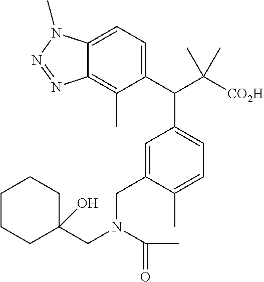 [SMILES: CC(=O)N(Cc1cc(C(c2ccc3c(nnn3C)c2C)C(C)(C)C(=O)O)ccc1C)CC1(O)CCCCC1]

To the mixture of methyl 3-(3-(chloromethyl)-4-methylphenyl)-3-(1,4-dimethyl-1H-benzo[d][1,2,3]triazol-5-yl)-2,2-dimethylpropanoate (60 mg, 0.150 mmol) in acetonitrile (3.0 mL) and tetrahydrofuran (1.0 mL) was added 1-(aminomethyl)cyclohexanol hydrochloride (49.7 mg, 0.300 mmol) and DIEA (0.105 mL, 0.600 mmol). The resulting reaction mixture was heated via microwave at 100° C. for 1 h twice before being concentrated under reduced pressure and the residue redissolved in methanol (2.0 mL). NaOH (3.0 N) (0.400 mL, 1.200 mmol) was added. The resulting mixture was heated via microwave at 130° C. for 1 h twice then acidified with HCl (3.0 N) (0.400 mL, 1.200 mmol), concentrated under reduced pressure and extracted with DCM (2×3 mL). The organic portion was dried over Na₂SO₄, filtered, and concentrated under reduced pressure. This residue was dissolved in dichloromethane (2.0 mL) before adding DIEA (0.105 mL, 0.600 mmol) and AcCl (0.021 mL, 0.300 mmol). The resulting reaction mixture was stirred at ambient temperature for 30 min before adding more AcCl (0.021 mL, 0.300 mmol) and DIEA (0.105 mL, 0.600 mmol). The resulting reaction mixture was stirred at ambient temperature for 17 h then quenched with H₂O (0.3 mL), concentrated under reduced pressure, and purified by reverse phase HPLC (formic acid modifier) to give the title compound (16.6 mg, 0.032 mmol, 21.25% yield). LC-MS m/z 521.4 (M+H)⁺, 1.01 (ret. time).

### Example 27

### 3-(3-((N-(Cycloheptylmethyl)acetamido)methyl)-4-methylphenyl)-3-(1,4-dimethyl-1H-benzo[d][1,2,3]triazol-5-yl)propanoic acid

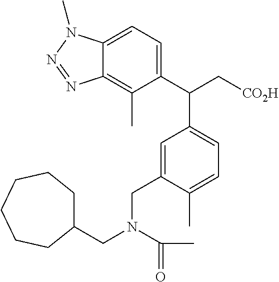 [SMILES: CC(=O)N(Cc1cc(C(CC(=O)O)c2ccc3c(nnn3C)c2C)ccc1C)CC1CCCCCC1]

To the mixture of ethyl 3-(3-(chloromethyl)-4-methylphenyl)-3-(1,4-dimethyl-1H-benzo[d][1,2,3]triazol-5-yl)propanoate (80 mg, 0.207 mmol) in acetonitrile (3.0 mL) and tetrahydrofuran (1.0 mL) was added cycloheptylmethanamine (0.060 mL, 0.415 mmol) and DIEA (0.145 mL, 0.829 mmol). The resulting reaction mixture was heated via microwave at 100° C. for 1 h before adding AcCl (0.029 mL, 0.415 mmol). The resulting reaction mixture was stirred at ambient temperature for 30 min then concentrated under reduced pressure and the residue redissolved in methanol (2.0 mL) followed by addition of NaOH (3.0 N) (0.553 mL, 1.659 mmol). The resulting reaction mixture was heated via microwave at 80° C. for 20 min before acidifying with HCl (3.0 N) (0.553 mL, 1.659 mmol). The reaction mixture was concentrated under reduced pressure, purified by reverse phase HPLC (formic acid modifier) to give the title compound (54.5 mg, 0.111 mmol, 53.6% yield). LC-MS m/z 491.4 (M+H)⁺, 1.12 (ret. time).

1. A compound of Formula (I): 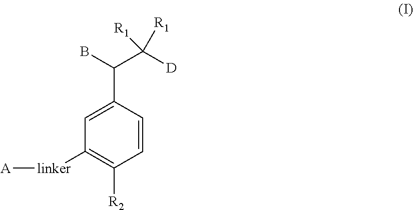
B is benzotriazolyl, phenyl, triazolopyridinyl, or —(CH₂)₂ triazolyl, each of which may be unsubstituted or by 1, 2, or 3 substituents independently chosen from:
—C₁₋₃alkyl, —O—C₁₋₃alkyl, CN, —(CH₂)₂—O—(CH₂)₂—OR₄ and halo;
D is —C(O)OH, —C(O)NHSO₂CH₃, —SO₂NHC(O)CH₃, 5-(trifluoromethyl)-4H-1,2,4-triazol-2-yl, or tetrazolyl;
R₁ is independently hydrogen, C₁alkyl, F, C₃₋₆spirocycloalkyl, oxetane, or the two R₁ groups together with the carbon they are attached to form a cyclopropyl group;
R₂ is hydrogen, methyl, CF₃, or halo;
Linker is 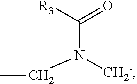
—O—C(O)—N(CH₃)—CH₂—,—C(O)—NH—CH₂— or —N(CH₃)—C(O)—CH₂—O—;
R₃ is CH₃, —(CH₂)₂—OH, or NH₂;
R₄ is hydrogen or C₁₋₃ alkyl;
A is cyclopentyl, cyclohexyl, cycloheptyl, or phenyl each of which may be substituted by one or two of —C₁₋₃ alkyl, CN, halo, —OH, or —O—C₁₋₃ alkyl groups;
or A is C₁₋₅ alkyl which may be substituted by —OCH₃;
and phenyl may also be substituted by —O—CH(CH₃)—C(O)—OH— or NO₂;
or a pharmaceutically acceptable salt thereof.

2. The compound of claim 1 wherein:
B is benzotriazolyl or —(CH₂)₂ triazolyl, each of which may be substituted or unsubstituted by 1, 2, or 3 substituents independently chosen from:
—C₁₋₃alkyl and halo;
D is —C(O)OH;
R₁ is independently hydrogen or methyl or the two R₁ groups together with the carbon they are attached to form a cyclopropyl group;
R₂ is methyl or chloro;
Linker is 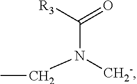
—O—C(O)—N(CH₃)—CH₂ or —N(Me)-C(O)—CH₂—O—;
R₃ is CH₃, —(CH₂)₂—OH, or NH₂;
A is cyclopentyl or cyclohexyl, each of which may be substituted independently by one or two C₁₋₃ alkyl;
or a pharmaceutically acceptable salt thereof.

3. The compound of claim 1 wherein:
B is benzotriazolyl which may be substituted or unsubstituted by 1, 2, or 3 substituents independently chosen from:
—C₁₋₃alkyl and/or —O—C₁₋₃alkyl;
D is —C(O)OH;
R₁ is hydrogen;
R₂ is methyl or halo;
Linker is 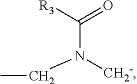
—O—C(O)—N(CH₃)—CH₂—, or —N(CH₃)—C(O)—CH₂—O—;
R₃ is CH₃, —(CH₂)₂—OH, or NH₂;
A is cyclopentyl, cyclohexyl, cycloheptyl, or phenyl each of which may be substituted independently by one or two of —C₁₋₃ alkyl, CN, halo, —OH, or —O—C₁₋₃ alkyl groups;
or A is —C₁₋₅ alkyl which may be substituted by —OCH₃;
and, wherein A is phenyl, it may also be substituted independently by —O—CH(CH₃)—C(O)—OH— or NO₂;
or a pharmaceutically acceptable salt thereof.

4. A compound which is:
3-[3-({[(tert-butoxy)carbonyl](methyl)amino}methyl)-4-chlorophenyl]-3-(1,4-dimethyl-1H-1,2,3-benzotriazol-5-yl)propanoic acid;
3-[4-chloro-3-({[(cyclopentyloxy)carbonyl](methyl)amino}methyl)phenyl]-3-(1-methyl-1H-1,2,3-benzotriazol-5-yl)propanoic acid;
3-[3-({[(butan-2-yloxy)carbonyl](methyl)amino}methyl)-4-chlorophenyl]-3-(1-methyl-1H-1,2,3-benzotriazol-5-yl)propanoic acid;
3-[3-({[(tert-butoxy)carbonyl](methyl)amino}methyl)-4-methylphenyl]-3-(7-methoxy-1methyl-1H-1,2,3-benzotriazol-5-yl)propanoic acid;
3-[4-chloro-3-({[(cyclohexyloxy)carbonyl](methyl)amino}methyl)phenyl]-3-(1-methyl-1H-1,2,3-benzotriazol-5-yl)propanoic acid;
3-[3-({[(tert-butoxy)carbonyl](methyl)amino}methyl)-4-chlorophenyl]-3-(7-methoxy-1-methyl-1H-1,2,3-benzotriazol-5-yl)propanoic acid;
3-{3-[(dimethylcarbamoyl)methoxy]-4-methylphenyl}-3-(1-methyl-1H-1,2,3-benzotriazol-5-yl)propanoic acid;
3-(1,4-dimethyl-1H-1,2,3-benzotriazol-5-yl)-3-(4-methyl-3-{[methyl(4nitrophenoxycarbonyl)amino]methyl}phenyl)propanoic acid;
3-[4-chloro-3-({[(cyclopentyloxy)carbonyl](methyl)amino}methyl)phenyl]-3-(7-methoxy -1-methyl-1H-1,2,3-benzotriazol-5-yl)propanoic acid;
3-(1,4-dimethyl-1H-1,2,3-benzotriazol-5-yl)-3-{3-[({[(1-methoxy-2-methylpropan-2-yl)oxy]carbonyl}(methyl)amino)methyl]-4-methylphenyl}propanoic acid;
3-(3-{[N-(cyclohexylmethyl)acetamido]methyl}-4-methylphenyl)-3-(1,4-dimethyl-1H-1,2,3-benzotriazol-5-yl)propanoic acid;
3-{3-[(N-benzylacetamido)methyl]-4-methylphenyl}-3-(1,4-dimethyl-1H-1,2,3-benzotriazol-5-yl)propanoic acid;
3-(1,4-dimethyl-1H-1,2,3-benzotriazol-5-yl)-3-[4-methyl-3-({N-[(3-methylphenyl)methyl]acetamido}methyl)phenyl]propanoic acid;
3-(1,4-dimethyl-1H-1,2,3-benzotriazol-5-yl)-3-[3-({N-[(2,3-dimethylphenyl)methyl]acetamido}methyl)-4-methylphenyl]propanoic acid;
3-(1,4-dimethyl-1H-1,2,3-benzotriazol-5-yl)-3-[3-({N-[(4-methoxyphenyl)methyl]acetamido}methyl)-4-methylphenyl]propanoic acid;
3-(1,4-dimethyl-1H-1,2,3-benzotriazol-5-yl)-3-[3-({N-[(4-ethylphenyl)methyl]acetamido}methyl)-4-methylphenyl]propanoic acid;
3-(1,4-dimethyl-1H-1,2,3-benzotriazol-5-yl)-3-[3-({N-[(4-ethylcyclohexyl)methyl]acetamido}methyl)-4-methylphenyl]propanoic acid;
3-(3-{[carbamoyl(cyclohexylmethyl)amino]methyl}-4-methylphenyl)-3-(1,4-dimethyl-1H-1,2,3-benzotriazol-5-yl)propanoic acid;
3-[3-({carbamoyl[(4-ethylcyclohexyl)methyl]amino}methyl)-4-methylphenyl]-3-(1,4-dimethyl-1H-1,2,3-benzotriazol-5-yl)propanoic acid;
3-(3-{[N-(cyclohexylmethyl)acetamido]methyl}-4-methylphenyl)-3-(7-methoxy-1-methyl-1H-1,2,3-benzotriazol-5-yl)propanoic acid;
2-{2-[({5-[2-carboxy -1-(1,4-dimethyl-1H-1,2,3-benzotriazol-5-yl)ethyl]-2-methylphenyl}methyl)carbamoyl]phenoxyl}propanoic acid;
3-(1,4-Dimethyl-1H-benzo[d][1,2,3]triazol-5-yl)-3-(3((1((4-ethylcyclohexyl)methyl)ureido)methyl)-4-methylphenyl)-2,2-dimethylpropanoic acid;
(S)-3-(3-((1-(Cycloheptylmethyl)ureido)methyl)-4-methylphenyl)-3-(1,4-dimethyl-1H-benzo[d][1,2,3]triazol-5-yl)-2,2-dimethylpropanoic acid;
(S)-3-(1,4-Dimethyl-1H-benzo[d][1,2,3]triazol-5-yl)-3-(3-((1-((4-ethyl-1-hydroxycyclohexyl)methyl)ureido)methyl)-4-methylphenyl)-2,2-dimethylpropanoic acid;
(S)-3-(1,4-Dimethyl-1H-benzo[d][1,2,3]triazol-5-yl)-3-(3-((1-((1-hydroxycycloheptyl)methyl)ureido)methyl)-4-methylphenyl)-2,2-dimethylpropanoic acid;
3-(1,4-Dimethyl-1H-benzo[d][1,2,3]triazol-5-yl)-3-(3-(N-((1-hydroxycyclohexyl)methyl)acetamido)methyl)-4-methylphenyl)-2,2-dimethylpropanoic acid; or 3-(3-((N-(Cycloheptylmethyl)acetamido)methyl)-4-methylphenyl)-3-(1,4-dimethyl-1H-benzo[d][1,2,3]triazol-5-yl)propanoic acid;
or a pharmaceutically acceptable salt thereof.

5. A pharmaceutical composition comprising a compound of claim 1 and a pharmaceutically acceptable carrier or excipient.

6. A method of treating respiratory and non-respiratory disorders, including COPD, asthma, fibrosis, chronic asthma, acute asthma, lung disease secondary to environmental exposures, acute lung infection, chronic lung infection, α1 antitrypsin disease, cystic fibrosis, autoimmune diseases, diabetic nephropathy, chronic kidney disease, sepsis-induced acute kidney injury, acute kidney injury (AKI), kidney disease or malfunction seen during kidney transplantation, Pulmonary Arterial Hypertension, atherosclerosis, hypertension, heart failure, Parkinson's disease (PD), Alzheimer's disease (AD), Friedreich's Ataxia (FA), amyotrophic lateral sclerosis (ALS), multiple sclerosis (MS), inflammatory bowel disease, colon cancer, neovascular (dry) AMD and neovascular (wet) AMD, eye injury, Fuchs Endothelial Corneal Dystrophy (FECD), uveitis or other inflammatory eye conditions, Non-alcoholic Steatohepatitis (NASH), toxin-induced liver disease (e.g., acetaminophen-induced hepatic disease), viral hepatitis, cirrhosis, psoriasis, dermatitis/topical effects of radiation, immunosuppression due to radiation exposure, Preeclampsia, and high altitude sickness, which comprises administering to a human in need thereof, a compound of claim 1.

7. A method according to claim 6 wherein the compound is administered orally.

8. A method according to claim 6 wherein the compound is administered intravenously.

9. A method according to claim 6 wherein the compound is administered by inhalation.

10. A method according to claim 6 wherein the disease is COPD.

11-13. (canceled)
